(19) United States
(12) Patent Application Publication
(10) Pub. No.: US 2011/0023150 A1
(43) Pub. Date: Jan. 27, 2011

(54) GENOME EDITING OF GENES ASSOCIATED WITH SCHIZOPHRENIA IN ANIMALS

(75) Inventors: Edward Weinstein, St. Louis, MO (US); Xiaoxia Cui, St. Louis, MO (US); Phil Simmons, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,980

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. G01N 33/00; A01K 67/00; C12N 5/10; C12N 9/16; C07H 21/04

(52) U.S. Cl. 800/3; 800/13; 800/15; 800/16; 800/17; 800/14; 435/325; 435/351; 435/350; 435/366; 435/363; 435/352; 435/196; 536/23.2

(57) ABSTRACT

The present invention provides genetically modified animals and cells comprising edited chromosomal sequences associated with schizophrenia. In particular, the animals or cells are generated using a zinc finger nuclease-mediated editing process. The invention also provides zinc finger nucleases that target chromosomal sequence associated with schizophrenia and the nucleic acids encoding said zinc finger nucleases. Also provided are methods of assessing the effects of agents in genetically modified animals and cells comprising edited chromosomal sequences associated with schizophrenia.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified animals or cells comprising at least one edited chromosomal sequence associated with schizophrenia. In particular, the invention relates to the use of a zinc finger nuclease-mediated process to edit chromosomal sequences associated with schizophrenia.

### BACKGROUND OF THE INVENTION

Schizophrenia is a severe mental illness characterized by disintegration of the process of thinking, contact with reality, and emotional responsiveness. The disease usually manifests as auditory hallucinations, paranoid or bizarre delusions, or disorganized speech and thinking. These manifestations typically lead to significant social and occupational dysfunction. Patients with schizophrenia typically have increased dopamine activity in the mesolimbic pathway of the brain. The main treatment is antipsychotic medication, which depresses dopamine activity. The treatment for schizophrenia is controversial and there is no adequate way to determine if the treatment is needed in an individual or if the treatment is having a beneficial effect.

The vast majority of drugs (approximately 91%) fail to successfully proceed through the three phases of drug testing in humans. A majority of those drugs fail to complete all phases of drug testing because of unforeseen toxicology in human patients, despite the fact that all of these drugs had been tested in animal models and were found to be safe. This is because toxicology testing is performed in animals, and animal proteins differ from the orthologous proteins in humans.

What is needed in the art are animals that are mutated for the genes involved in schizophrenia processes, including knockouts, multiple mutant lines (double knockouts, triple knockouts, etc.) and/or over-expression of alleles that either cause disease or are associated with disease in humans, as well as “humanized” animals that express or over-express human homologues of relevant genes in animals.

### SUMMARY OF THE INVENTION

One aspect of the present disclosure encompasses a genetically modified animal comprising at least one edited chromosomal sequence encoding a protein associated with schizophrenia.

A further aspect provides a non-human embryo comprising at least one RNA molecule encoding a zinc finger nuclease that recognizes a chromosomal sequence encoding a protein associated with schizophrenia, and, optionally, at least one donor polynucleotide comprising a sequence encoding an ortholog of the protein associated with schizophrenia or an edited protein associated with schizophrenia.

An additional aspect provides a genetically modified cell comprising at least one edited chromosomal sequence encoding a protein associated with schizophrenia.

An alternate aspect provides a zinc finger nuclease comprising (a) a zinc finger DNA binding domain that binds a sequence having at least about 80% sequence identity to a sequence chosen from SEQ ID NOs: 4, 5, 6, and 7 and (b) a cleavage domain.

Another aspect provides a nucleic acid sequence that is recognized by a zinc finger nuclease. The nucleic acid sequence has at least about 80% sequence identity with a sequence chosen from SEQ ID NOs: 4, 5, 6, and 7.

Yet another aspect encompasses a method for assessing the effect of an agent in a genetically modified animal. The method comprises administering the agent to the genetically modified animal comprising at least one edited chromosomal sequence encoding a protein associated with schizophrenia, and comparing a parameter obtained from the genetically modified animal to the parameter obtained from a wild-type animal administered the same agent. The selected parameter is chosen from (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); and (g) ability of the agent to modify an incidence or indication of a schizophrenia-related disorder in the genetically modified animal.

Still yet another aspect encompasses a method for assessing the therapeutic potential of an agent as a treatment for a schizophrenia-related disorder. The method comprises administering the agent to a genetically modified animal, wherein the genetically modified animal comprises at least one edited chromosomal sequence encoding a protein associated with schizophrenia, and comparing a selected parameter obtained from the genetically modified animal to the selected parameter obtained from a wild-type animal with no exposure to the same agent. The selected parameter is chosen from: a) spontaneous behaviors; b) performance during behavioral testing; c) physiological anomalies; d) abnormalities in tissues or cells; e) biochemical function; and f) molecular structures.

Other aspects and features of the disclosure are described more thoroughly below.

### REFERENCE TO COLOR FIGURES

The application file contains at least one figure executed in color. Copies of this patent application publication with color figures will be provided by the Office upon request and payment of the necessary fee.

### BRIEF DESCRIPTION OF THE FIGURES

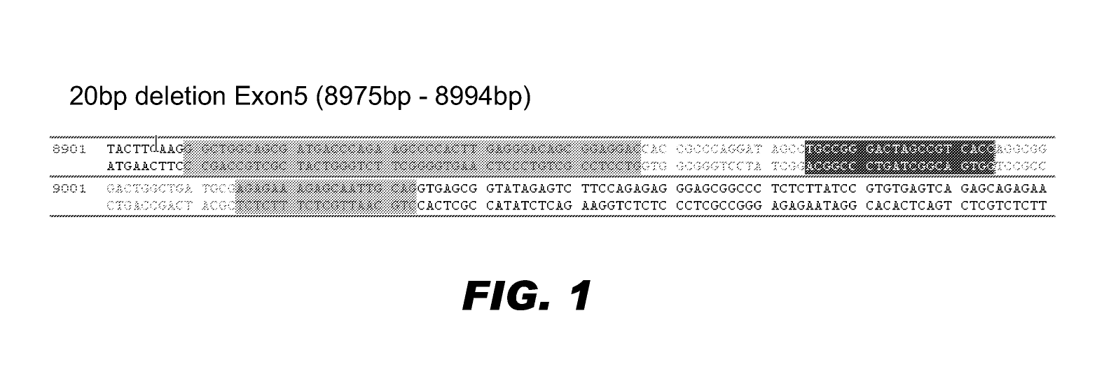

FIG. 1 presents the DNA sequence of an edited DISC1 locus. Presented is a region of the rat DISC1 (SEQ ID NO:1) in which there is a 20 bp deletion in the target sequence in exon 5. The exon is shown in green; the target site is presented in yellow, and the deletion is shown in dark blue.

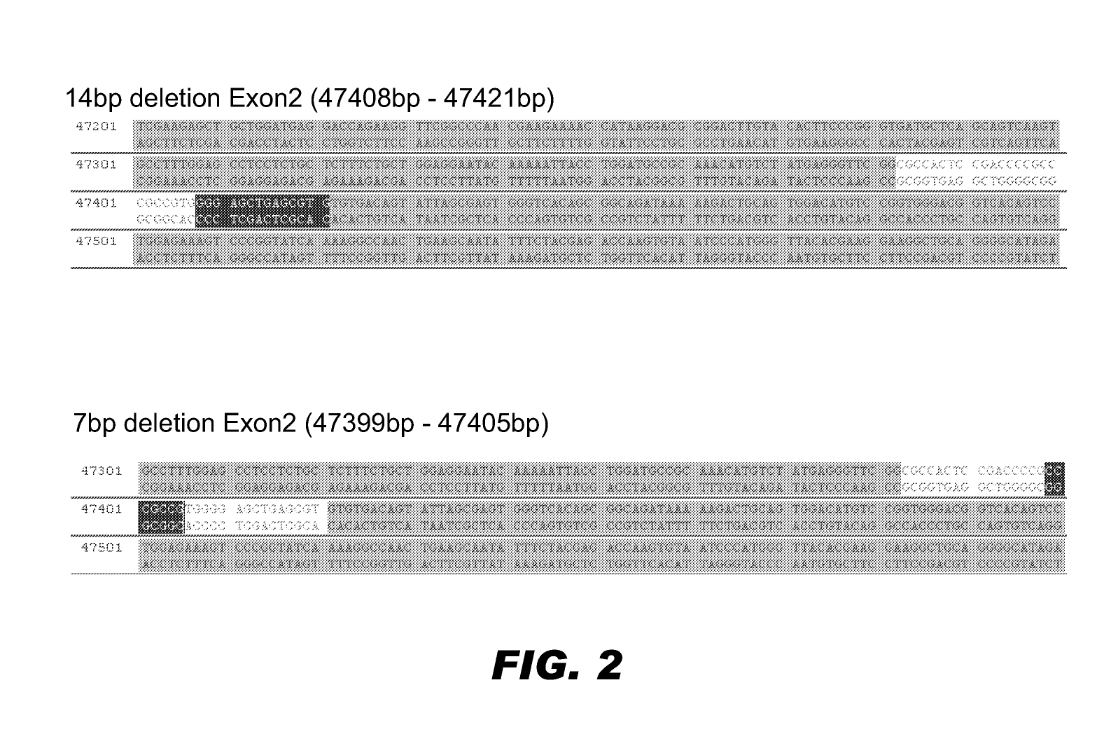

FIG. 2 shows the DNA sequences of edited BDNF loci in two animals. The upper sequence (SEQ ID NO:2) has a 14 bp deletion in the target sequence in exon 2, and the lower sequence (SEQ ID NO:3) has a 7 bp deletion in the target sequence in exon 2. The exon is shown in green; the target site is presented in yellow, and the deletions are shown in dark blue.

### DETAILED DESCRIPTION OF THE INVENTION

The present disclosure provides a genetically modified animal or animal cell comprising at least one edited chromosomal sequence encoding a protein associated with schizophrenia. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein associated with schizophrenia generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with schizophrenia using targeted zinc finger nuclease technology is rapid, precise, and highly efficient.

### (I) Genetically Modified Animals

One aspect of the present disclosure provides a genetically modified animal in which at least one chromosomal sequence associated with schizophrenia has been edited. For example, the edited chromosomal sequence may be inactivated such that the sequence is not transcribed and/or a functional protein encoded by a chromosomal sequence associated with schizophrenia is not produced. Alternatively, the edited chromosomal sequence may be modified such that it codes for an altered protein associated with schizophrenia. For example, the chromosomal sequence may be modified such that at least one nucleotide is changed and the expressed protein associated with schizophrenia comprises at least changed amino acid residue. The modified protein associated with schizophrenia may have altered substrate specificity, altered enzyme activity, altered kinetic rates, and so forth. Furthermore, the edited chromosomal sequence encoding a protein associated with schizophrenia may comprise an integrated sequence and/or a sequence encoding a protein associated with schizophrenia that may be integrated into the genome of the animal. The genetically modified animal disclosed herein may be heterozygous for the edited chromosomal sequence associated with schizophrenia. Alternatively, the genetically modified animal may be homozygous for the edited chromosomal sequence associated with schizophrenia.

In one embodiment, the genetically modified animal may comprise at least one inactivated chromosomal sequence associated with schizophrenia. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional protein associated with schizophrenia is not produced. The inactivated chromosomal sequence comprises no exogenously introduced sequence. Such an animal may be termed a “knockout.” Also included herein are genetically modified animals in which two, three, or more chromosomal sequences associated with schizophrenia are inactivated.

In another embodiment, the edited chromosomal sequence may be modified such that it codes for an altered protein associated with schizophrenia. The chromosomal sequence may be modified such that at least one nucleotide is changed and the expressed protein associated with schizophrenia comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed protein associated with schizophrenia comprises a single amino acid deletion or insertion, provided such a protein is functional. The modified protein associated with schizophrenia may have altered substrate specificity, altered enzyme activity, altered kinetic rates, and so forth. In some embodiments, the modified protein associated with schizophrenia comprises at least one modification such that the altered version of the protein causes schizophrenia. In other embodiments, the modified protein associated with schizophrenia comprises at least one modification such that the altered version of the protein associated with schizophrenia protects against schizophrenia.

In a further embodiment, the genetically modified animal may comprise at least one chromosomally integrated sequence encoding a protein associated with schizophrenia. For example, an exogenous sequence encoding an orthologous or an endogenous protein associated with schizophrenia may be integrated into a chromosomal sequence encoding a protein associated with schizophrenia such that the chromosomal sequence is inactivated, but wherein the exogenous sequence encoding the orthologous or endogenous protein associated with schizophrenia may be expressed. In such a case, the sequence encoding the orthologous or endogenous protein associated with schizophrenia may be operably linked to a promoter control sequence. The exogenous sequence encoding the orthologous or endogenous protein associated with schizophrenia may be such that the protein associated with schizophrenia is over-produced, or the tissue-specific or temporal expression of the protein associated with schizophrenia is altered, or a combination thereof. Alternatively, an exogenous sequence encoding an orthologous or endogenous protein associated with schizophrenia may be integrated into a chromosomal sequence without affecting expression of a chromosomal sequence. For example, an exogenous sequence encoding a protein associated with schizophrenia may be integrated into a “safe harbor” locus, such as the Rosa26 locus, HPRT locus, or AAV locus, wherein the exogenous sequence encoding the orthologous or endogenous protein associated with schizophrenia may be expressed or over-expressed. In one iteration of the disclosure an animal comprising a chromosomally integrated sequence encoding a protein associated with schizophrenia may be called a “knock-in”, and it should be understood that in such an iteration of the animal, no selectable marker is present. The present disclosure also encompasses genetically modified animals in which 2, 3, 4, 5, 6, 7, 8, 9 or more sequences encoding proteins associated with schizophrenia are integrated into the genome.

The chromosomally integrated sequence encoding a protein associated with schizophrenia may encode the wild type form of the protein associated with schizophrenia. Alternatively, the chromosomally integrated sequence encoding a protein associated with schizophrenia may comprise at least one modification such that an altered version of the protein associated with schizophrenia is produced. In some embodiments, the chromosomally integrated sequence encoding a protein associated with schizophrenia comprises at least one modification such that the altered version of the protein causes schizophrenia. In other embodiments, the chromosomally integrated sequence encoding a protein associated with schizophrenia comprises at least one modification such that the altered version of the protein associated with schizophrenia protects against schizophrenia.

In an additional embodiment, the genetically modified animal may be a “humanized” animal comprising at least one chromosomally integrated sequence encoding a functional human protein associated with schizophrenia. The functional human protein associated with schizophrenia may have no corresponding ortholog in the genetically modified animal. Alternatively, the wild-type animal from which the genetically modified animal is derived may comprise an ortholog corresponding to the functional human protein associated with schizophrenia. In this case, the orthologous sequence in the “humanized” animal is inactivated such that no functional protein is made and the “humanized” animal comprises at least one chromosomally integrated sequence encoding the human protein associated with schizophrenia. For example, a humanized animal may comprise an inactivated abat sequence and a chromosomally integrated human ABAT sequence. Those of skill in the art appreciate that “humanized” animals may be generated by crossing a knock out animal with a knock in animal comprising the chromosomally integrated sequence.

In yet another embodiment, the genetically modified animal may comprise at least one edited chromosomal sequence encoding a protein associated with schizophrenia such that the expression pattern of the protein is altered. For example, regulatory regions controlling the expression of the protein, such as a promoter or transcription binding site, may be altered such that the protein associated with schizophrenia is over-produced, or the tissue-specific or temporal expression of the protein is altered, or a combination thereof. Alternatively, the expression pattern of the protein associated with schizophrenia may be altered using a conditional knockout system. A non-limiting example of a conditional knockout system includes a Cre-lox recombination system. A Cre-lox recombination system comprises a Cre recombinase enzyme, a site-specific DNA recombinase that can catalyze the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a protein associated with schizophrenia. The genetically modified animal comprising the lox-flanked chromosomal sequence encoding a protein associated with schizophrenia may then be crossed with another genetically modified animal expressing Cre recombinase. Progeny animals comprising the lox-flanked chromosomal sequence and the Cre recombinase are then produced, and the lox-flanked chromosomal sequence encoding a protein associated with schizophrenia is recombined, leading to deletion or inversion of the chromosomal sequence encoding the protein. Expression of Cre recombinase may be temporally and conditionally regulated to effect temporally and conditionally regulated recombination of the chromosomal sequence encoding a protein associated with schizophrenia.

(a) Chromosomal Sequences and Proteins Associated with Schizophrenia

The present disclosure comprises editing of any chromosomal sequences that encode proteins associated with schizophrenia. The proteins associated with schizophrenia are typically selected based on an experimental association of the protein associated with schizophrenia to the development or progression of schizophrenia. For example, the production rate or circulating concentration of a protein associated with schizophrenia may be elevated or depressed in a population having schizophrenia relative to a population not having schizophrenia. Differences in protein levels may be assessed using proteomic techniques including but not limited to Western blot, immunohistochemical staining, enzyme linked immunosorbent assay (ELISA), and mass spectrometry. Alternatively, the proteins associated with schizophrenia may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR). Exemplary non-limiting examples of chromosomal sequences associated with schizophrenia include NRG1, ErbB4, CPLX1, TPH1, TPH2, NRXN1, GSK3A, BDNF, DISC1, GSK3B, and combinations thereof, each of which is described in more detail below.

(i) NRG1 and ErbB4

Neuregulin 1 is a protein that is encoded by the NRG1 gene. NGR1 is one of four proteins in the neuregulin family that act on the EGFR family of receptors and is produced in numerous isoforms by alternative splicing, which allows it to perform a wide variety of functions. Neuregulin 1 is essential for the development of the nervous system and the heart. It is thought that Neuregulin 1-ErbB4 interactions play a role in the pathological mechanism of schizophrenia. Receptor tyrosine-protein kinase ErbB4 is an enzyme that is encoded by the ErbB4 gene. The ErbB4 gene encodes a receptor tyrosine kinase. NRG1 has also been identified as a candidate for bipolar disorder.

(ii) CPLX1

Complexin 1 is a protein encoded by the CPLX1 gene. Proteins encoded by the complexin/synaphin gene family are cystolic proteins that function in synaptic vesicle exocytosis. CPLX1 has been shown to interact with SNAP-25 and STX1A.

(iii) TPH1 and TPH2

TPH1, tryptophan hydroxylase 1 is an enzyme encoded by the TPH1 gene. TPH2 encodes tryptophan hydroxylase 2, another tryptophan hydroxylase. Tryptophan hydroxylases catalyze the biopterin-dependent monooxygenation of tryptophan to 5-hydroxytryptophan, which is subsequently decarboxylated to form the neurotransmitted serotonin. The expression of TPH1 and TPH2 is limited to the raphe neurons, pinealocytes, mast calls, mononuclear leukocytes, beta cells of the islets of Langerhans, and intestinal and pancreatic enterochromaffin cells. Tryptophan hydroxlases are important for the brain, even thought they are not detected there. The effect of variations of the TPH1 gene on brain related variables, such as personality traits and neuropsychiatric disorders has been studied. A human mutant of TPH1, A218C found in intron 7, is highly associated with schizophrenia. The correlation of an intron mutant with schizophrenia is significant because it suggests that introns have an important role in translation, transcription, or other aspects of the production of proteins from DNA.

(iv) NRXN1

Neurexin-1-alpha is a protein that is encoded by the NRXN1 gene. Neurexins are a family of proteins that function as cell adhesion molecules and receptors. The Neurexin genes are very large and occupy up to 2% of human chromosome 14. Little is known about Neurexin mutations that lead to diseases and disorders in humans.

(v) GSK3A

Glycogen synthase kinase-3 alpha is an enzyme encoded by the GSK3A gene. Glycogen synthase kinase-3 alpha is a multifunctional protein serine kinase implicated in the control of several regulatory proteins such as glycogen synthase and various transcription factors, as well as playing a role in the WNT and phosphoinositide 3-kinase signaling pathways.

(vi) GSK3B

Glycogen synthase kinase-3 beta is an enzyme encoded by the GSK3B gene. Glycogen synthase kinase-3 beta is a proline-directed serine-threonine kinase. GSK3B is involved in energy metabolism, neuronal cell development, and body pattern formation. GSK3B is involved in energy metabolism, neuronal cell development, and body pattern formation. Polymorphisms in this gene have been implicated in modifying risk of Parkinson disease and Alzheimer disease.

(vii) BNDF

BDNF or brain-derived neurotrophic factor is encoded by the BDNF gene. BDNF is a member of the “neurotrophin” family of growth factors and are found in the brain and the periphery. BDNF acts on certain neurons of the central nervous system and the peripheral nervous system to help support the survival of existing neurons and encourage the growth and differentiation of new neurons and synapses. BDNF is active in the hippocampus, cortex, and basal forebrain. BDNF binds at least two receptors on the surface of cells that are capable of responding to this growth factor, TrkB and the LNGFR (low-affinity nerve growth factor receptor), as well as modulating the activity of various neurotransmitter receptors. It has been shown that BDNF is possibly linked to depression, schizophrenia, obsessive-compulsive disorder, Alzheimer's disease, Huntington's disease, Rett syndrome, dementia, anorexia nervosa, and bullemia nervosa. Additionally, BDNF is thought to have a role is depression, eczema, and epilepsy.

(viii) DISC1

DISC1 or disrupted in schizophrenia 1 is a gene that encodes a protein with multiple coiled coil motifs, which is located in the nucleus, cytoplasm, and mitochondria of the cell. DISC1 is involved in neurite outgrowth and cortical development through its interaction with other proteins. A disruption in a t(1;11)(q42.1;q14.3) translocation that segregates with schizophrenia and related psychiatric disorders. DISC1 is required for neural progenitor proliferation in the ventrical/subventrial zone during embryonic brain development and in the adult dentate gyrus of the hippocampus. Genetic variation in DISC1 is associated with susceptibility to schizophrenia type 9.

In additional iterations, the chromosomal sequence may comprise, but is not limited to, TP53 (tumor protein p53), IL1B (interleukin 1, beta), IL4 (interleukin 4), BCL2 (B-cell CLL/lymphoma 2), GRIN1 (glutamate receptor, ionotropic, N-methyl D-aspartate 1), CCND1 (cyclin D1), BAX (BCL2-associated X protein), KCNN3 (potassium intermediate/small conductance calcium-activated channel, subfamily N, member 3), SNAP25 (synaptosomal-associated protein, 25 kDa), GRIK3 (glutamate receptor, ionotropic, kainate 3), GRIK1 (glutamate receptor, ionotropic, kainate 1), GRIK2 (glutamate receptor, ionotropic, kainate 2), GRIK5 (glutamate receptor, ionotropic, kainate 5), PDGFRB (platelet-derived growth factor receptor, beta polypeptide), ASCL1 (achaete-scute complex homolog 1 (Drosophila)), GRIK4 (glutamate receptor, ionotropic, kainate 4), D102 (deiodinase, iodothyronine, type II), XDH (xanthine dehydrogenase), CTSK (cathepsin K), MB (myoglobin), DBN1 (drebrin 1), CCND2 (cyclin D2), MC1R (melanocortin 1 receptor (alpha melanocyte stimulating hormone receptor)), CAPN10 (calpain 10), KCNJ9 (potassium inwardly-rectifying channel, subfamily J, member 9), KCNJ5 (potassium inwardly-rectifying channel, subfamily J, member 5), ACSL4 (acyl-CoA synthetase long-chain family member 4), BIK (BCL2-interacting killer (apoptosis-inducing)), PRDX2 (peroxiredoxin 2), BCL2A1 (BCL2-related protein A1), PRDX1 (peroxiredoxin 1), KCNN2 (potassium intermediate/small conductance calcium-activated channel, subfamily N, member 2), TNF (tumor necrosis factor (TNF superfamily, member 2)), INS (insulin), IL6 (interleukin 6 (interferon, beta 2)), DRD2 (dopamine receptor D2), AKT1 (v-akt murine thymoma viral oncogene homolog 1), COMT (catechol-O-methyltransferase), BDNF (brain-derived neurotrophic factor), IFNG (interferon, gamma), PRL (prolactin), HTR2A (5-hydroxytryptamine (serotonin) receptor 2A), NRG1 (neuregulin 1), TGFB1 (transforming growth factor, beta 1), IL10 (interleukin 10), IL2 (interleukin 2), APOE (apolipoprotein E), MAPK1 (mitogen-activated protein kinase 1), SLC6A4 (solute carrier family 6 (neurotransmitter transporter, serotonin), member 4), DRD3 (dopamine receptor D3), SOD1 (superoxide dismutase 1, soluble), IGF1 (insulin-like growth factor 1 (somatomedin C)), EGFR (epidermal growth factor receptor (erythroblastic leukemia viral (v-erb-b) oncogene homolog, avian)), TH (tyrosine hydroxylase), DTNBP1 (dystrobrevin binding protein 1), MTHFR (5,10-methylenetetrahydrofolate reductase (NADPH)), DRD4 (dopamine receptor D4), LEP (leptin), EGF (epidermal growth factor (beta-urogastrone)), PTGS2 (prostaglandin-endoperoxide synthase 2 (prostaglandin G/H synthase and cyclooxygenase)), NOS1 (nitric oxide synthase 1 (neuronal)), ACE (angiotensin I converting enzyme (peptidyl-dipeptidase A) 1), RELN (reelin), ALB (albumin), HLA-DRB1 (major histocompatibility complex, class II, DR beta 1), ESR1 (estrogen receptor 1), CRP (C-reactive protein, pentraxin-related), VEGFA (vascular endothelial growth factor A), SLC6A3 (solute carrier family 6 (neurotransmitter transporter, dopamine), member 3), GAD1 (glutamate decarboxylase 1 (brain, 67 kDa)), CTNNB1 (catenin (cadherin-associated protein), beta 1, 88 kDa), IL8 (interleukin 8), HLA-DQB1 (major histocompatibility complex, class II, DQ beta 1), ABCB1 (ATP-binding cassette, sub-family B (MDR/TAP), member 1), NGF (nerve growth factor (beta polypeptide)), ADIPOQ (adiponectin, C1Q and collagen domain containing), JUN (jun oncogene), RGS4 (regulator of G-protein signaling 4), DYT10 (dystonia 10), FOS (FBJ murine osteosarcoma viral oncogene homolog), CYP2D6 (cytochrome P450, family 2, subfamily D, polypeptide 6), SYP (synaptophysin), NOS2 (nitric oxide synthase 2, inducible), ACHE (acetylcholinesterase (Yt blood group)), MAPK8 (mitogen-activated protein kinase 8), CAT (catalase), ICAM1 (intercellular adhesion molecule 1), CNR1 (cannabinoid receptor 1 (brain)), ERBB2 (v-erb-b2 erythroblastic leukemia viral oncogene homolog 2, neuro/glioblastoma derived oncogene homolog (avian)), FN1 (fibronectin 1), DAO (D-amino-acid oxidase), IFNA1 (interferon, alpha 1), CASP3 (caspase 3, apoptosis-related cysteine peptidase), HTR1A (5-hydroxytryptamine (serotonin) receptor 1A), NPY (neuropeptide Y), FGF2 (fibroblast growth factor 2 (basic)), APP (amyloid beta (A4) precursor protein), MAPK14 (mitogen-activated protein kinase 14), CYP1A2 (cytochrome P450, family 1, subfamily A, polypeptide 2), NOS3 (nitric oxide synthase 3 (endothelial cell)), HTR3A (5-hydroxytryptamine (serotonin) receptor 3A), MMP9 (matrix metallopeptidase 9 (gelatinase B, 92 kDa gelatinase, 92 kDa type IV collagenase)), NTF3 (neurotrophin 3), CDKN2A (cyclin-dependent kinase inhibitor 2A (melanoma, p16, inhibits CDK4)), CCL2 (chemokine (C-C motif) ligand 2), AR (androgen receptor), MAPK10 (mitogen-activated protein kinase 10), MMP2 (matrix metallopeptidase 2 (gelatinase A, 72 kDa gelatinase, 72 kDa type IV collagenase)), URN (interleukin 1 receptor antagonist), GFAP (glial fibrillary acidic protein), PPARG (peroxisome proliferator-activated receptor gamma), PLA2G4A (phospholipase A2, group IVA (cytosolic, calcium-dependent)), POMC (proopiomelanocortin), IL1A (interleukin 1, alpha), TF (transferrin), EPO (erythropoietin), CCK (cholecystokinin), IL3 (interleukin 3 (colony-stimulating factor, multiple)), CYP3A4 (cytochrome P450, family 3, subfamily A, polypeptide 4), DBH (dopamine beta-hydroxylase (dopamine beta-monooxygenase)), TSC1 (tuberous sclerosis 1), IGF2 (insulin-like growth factor 2 (somatomedin A)), NTS (neurotensin), CD79A (CD79a molecule, immunoglobulin-associated alpha), CD40LG (CD40 ligand), GH1 (growth hormone 1), MBP (myelin basic protein), HLA-A (major histocompatibility complex, class I, A), VIM (vimentin), SOD2 (superoxide dismutase 2, mitochondrial), ERBB4 (v-erb-a erythroblastic leukemia viral oncogene homolog 4 (avian)), GSTM1 (glutathione S-transferase mu 1), RB1 (retinoblastoma 1), GAP43 (growth associated protein 43), GRIN2B (glutamate receptor, ionotropic, N-methyl D-aspartate 2B), S100B (S100 calcium binding protein B), MAP2 (microtubule-associated protein 2), CALB2 (calbindin 2), CCR5 (chemokine (C-C motif) receptor 5), PIK3CG (phosphoinositide-3-kinase, catalytic, gamma polypeptide), PAFAH1 B1 (platelet-activating factor acetylhydrolase 1b, regulatory subunit 1 (45 kDa)), PAX6 (paired box 6), FGFR1 (fibroblast growth factor receptor 1), IL2RA (interleukin 2 receptor, alpha), SST (somatostatin), DRD1 (dopamine receptor D1), F2 (coagulation factor II (thrombin)), GSK3B (glycogen synthase kinase 3 beta), CHRNA7 (cholinergic receptor, nicotinic, alpha 7), HLA-B (major histocompatibility complex, class I, B), HIF1A (hypoxia inducible factor 1, alpha subunit (basic helix-loop-helix transcription factor)), HTR2c (5-hydroxytryptamine (serotonin) receptor 2C), PPIG (peptidylprolyl isomerase G (cyclophilin G)), NCAM1 (neural cell adhesion molecule 1), HP (haptoglobin), NTRK2(neurotrophic tyrosine kinase, receptor, type 2), NR3C1 (nuclear receptor subfamily 3, group C, member 1 (glucocorticoid receptor)), MAOA (monoamine oxidase A), MMP3 (matrix metallopeptidase 3 (stromelysin 1, progelatinase)), GDNF (glial cell derived neurotrophic factor), HLA-DQA1 (major histocompatibility complex, class II, DQ alpha 1), PLA2G6 (phospholipase A2, group VI (cytosolic, calcium-independent)), TPH1 (tryptophan hydroxylase 1), IL18 (interleukin 18 (interferon-gamma-inducing factor)), ERBB3 (v-erb-b2 erythroblastic leukemia viral oncogene homolog 3 (avian)), DLG4 (discs, large homolog 4 (Drosophila)), SP1 (Sp1 transcription factor), LPL (lipoprotein lipase), IGHE (immunoglobulin heavy constant epsilon), PIK3CA (phosphoinositide-3-kinase, catalytic, alpha polypeptide), CALCA (calcitonin-related polypeptide alpha), RAC1 (ras-related C3 botulinum toxin substrate 1 (rho family, small GTP binding protein Rac1)), CREB1 (cAMP responsive element binding protein 1), CHAT (choline acetyltransferase), CSF3 (colony stimulating factor 3 (granulocyte)), ATM (ataxia telangiectasia mutated), CTSB (cathepsin B), GRB2 (growth factor receptor-bound protein 2), IGF1 R (insulin-like growth factor 1 receptor), GDF15 (growth differentiation factor 15), CNTF (ciliary neurotrophic factor), APOB (apolipoprotein B (including Ag(x) antigen)), APC (adenomatous polyposis coli), MAPT (microtubule-associated protein tau), CABIN1 (calcineurin binding protein 1), PTGS1 (prostaglandin-endoperoxide synthase 1 (prostaglandin G/H synthase and cyclooxygenase)), IL5 (interleukin 5 (colony-stimulating factor, eosinophil)), IGFBP3 (insulin-like growth factor binding protein 3), APOA1 (apolipoprotein A-I), PLA2G2A (phospholipase A2, group IIA (platelets, synovial fluid)), RHOA (ras homolog gene family, member A), NTRK3 (neurotrophic tyrosine kinase, receptor, type 3), CRH (corticotropin releasing hormone), INSR (insulin receptor), CD14 (CD14 molecule), HDAC9 (histone deacetylase 9), GPT (glutamic-pyruvate transaminase (alanine aminotransferase)), GSTT1 (glutathione S-transferase theta 1), MET (met proto-oncogene (hepatocyte growth factor receptor)), CALB1 (calbindin 1, 28 kDa), HSPG2 (heparan sulfate proteoglycan 2), PPARα (peroxisome proliferator-activated receptor alpha), NOTCH1 (Notch homolog 1, translocation-associated (Drosophila)), LIF (leukemia inhibitory factor (cholinergic differentiation factor)), VCAM1 (vascular cell adhesion molecule 1), RARA (retinoic acid receptor, alpha), NPPA (natriuretic peptide precursor A), GNB3 (guanine nucleotide binding protein (G protein), beta polypeptide 3), MAOB (monoamine oxidase B), GAPDH (glyceraldehyde-3-phosphate dehydrogenase), ACTB (actin, beta), PITX2 (paired-like homeodomain 2), UR1 (interleukin 1 receptor, type I), HSP90AA1 (heat shock protein 90 kDa alpha (cytosolic), class A member 1), GSTP1 (glutathione S-transferase pi 1), BCHE (butyrylcholinesterase), KDR (kinase insert domain receptor (a type III receptor tyrosine kinase)), GLUL (glutamate-ammonia ligase (glutamine synthetase)), GNRH1 (gonadotropin-releasing hormone 1 (luteinizing-releasing hormone)), REN (renin), CHKA (choline kinase alpha), VDR (vitamin D (1,25-dihydroxyvitamin D3) receptor), MAP2K1 (mitogen-activated protein kinase kinase 1), IRS1 (insulin receptor substrate 1), STN (statin), FYN (FYN oncogene related to SRC, FGR, YES), HSPA4 (heat shock 70 kDa protein 4), IL2RB (interleukin 2 receptor, beta), ESR2 (estrogen receptor 2 (ER beta)), VIP (vasoactive intestinal peptide), FGFR2 (fibroblast growth factor receptor 2), DNMT1 (DNA (cytosine-5-)-methyltransferase 1), CYP2C9 (cytochrome P450, family 2, subfamily C, polypeptide 9), NDEL1 (nudE nuclear distribution gene E homolog (A. nidulans)-like 1), ADCY10 (adenylate cyclase 10 (soluble)), TNFRSF1A (tumor necrosis factor receptor superfamily, member 1A), NFKB1 (nuclear factor of kappa light polypeptide gene enhancer in B-cells 1), SNCA (synuclein, alpha (non A4 component of amyloid precursor)), ENO2 (enolase 2 (gamma, neuronal)), CTLA4 (cytotoxic T-lymphocyte-associated protein 4), NR4A2 (nuclear receptor subfamily 4, group A, member 2), NTRK1 (neurotrophic tyrosine kinase, receptor, type 1), NQO1 (NAD(P)H dehydrogenase, quinone 1), EGR1 (early growth response 1), SLC2A1 (solute carrier family 2 (facilitated glucose transporter), member 1), VWF (von Willebrand factor), NEFL (neurofilament, light polypeptide), FEZ1 (fasciculation and elongation protein zeta 1 (zygin I)), ID01 (indoleamine 2,3-dioxygenase 1), ADCYAP1 (adenylate cyclase activating polypeptide 1 (pituitary)), MAG (myelin associated glycoprotein), CYP2C19 (cytochrome P450, family 2, subfamily C, polypeptide 19), CHRM1 (cholinergic receptor, muscarinic 1), B2M (beta-2-microglobulin), LTA (lymphotoxin alpha (TNF superfamily, member 1)), APOC3 (apolipoprotein CAI), XBP1 (X-box binding protein 1), PTK2 (PTK2 protein tyrosine kinase 2), MECP2 (methyl CpG binding protein 2 (Rett syndrome)), SLC1A2 (solute carrier family 1 (glial high affinity glutamate transporter), member 2), SHBG (sex hormone-binding globulin), KLK3 (kallikrein-related peptidase 3), GLB1 (galactosidase, beta 1), TNFRSF1 B (tumor necrosis factor receptor superfamily, member 1 B), IL6R (interleukin 6 receptor), CYP19A1 (cytochrome P450, family 19, subfamily A, polypeptide 1), TACR1 (tachykinin receptor 1), TGFBR2 (transforming growth factor, beta receptor II (70/80 kDa)), RNH1 (ribonuclease/angiogenin inhibitor 1), OLIG2 (oligodendrocyte lineage transcription factor 2), TNC (tenascin C), PRNP (prion protein), MAP1 B (microtubule-associated protein 1 B), GRIN2A (glutamate receptor, ionotropic, N-methyl D-aspartate 2A), GRM5 (glutamate receptor, metabotropic 5), FGF1 (fibroblast growth factor 1 (acidic)), PLP1 (proteolipid protein 1), KCND1 (potassium voltage-gated channel, ShaI-related subfamily, member 1), TAC1 (tachykinin, precursor 1), CAMK2G (calcium/calmodulin-dependent protein kinase II gamma), PDLIM5 (PDZ and LIM domain 5), GRIA2 (glutamate receptor, ionotropic, AMPA 2), HTR1B (5-hydroxytryptamine (serotonin) receptor 1 B), HSPD1 (heat shock 60 kDa protein 1 (chaperonin)), CDK5 (cyclin-dependent kinase 5), CHGA (chromogranin A (parathyroid secretory protein 1)), CREBBP (CREB binding protein), TCF7L2 (transcription factor 7-like 2 (T-cell specific, HMG-box)), ZDHHC8 (zinc finger, DHHC-type containing 8), DRD5 (dopamine receptor D5), ERVWE1 (endogenous retroviral family W, env(C7), member 1), DNMT3B (DNA (cytosine-5-)-methyltransferase 3 beta), ADRB3 (adrenergic, beta-3-, receptor), ADORA2A (adenosine A2a receptor), DPYSL2 (dihydropyrimidinase-like 2), CP (ceruloplasmin (ferroxidase)), TYR (tyrosinase (oculocutaneous albinism IA)), LEPR (leptin receptor), PSEN1 (presenilin 1), DES (desmin), TFRC (transferrin receptor (p90, CD71)), SCT (secretin), SEMA3A (sema domain, immunoglobulin domain (Ig), short basic domain, secreted, (semaphorin) 3A), RARB (retinoic acid receptor, beta), CYP2E1 (cytochrome P450, family 2, subfamily E, polypeptide 1), TTF2 (transcription termination factor, RNA polymerase II), TSPO (translocator protein (18 kDa)), PDYN (prodynorphin), RUNX1 (runt-related transcription factor 1), PRKCA (protein kinase C, alpha), GJA1 (gap junction protein, alpha 1, 43 kDa), SLC18A2 (solute carrier family 18 (vesicular monoamine), member 2), IL6ST (interleukin 6 signal transducer (gp130, oncostatin M receptor)), LOC646627 (phospholipase inhibitor), ADRA2A (adrenergic, alpha-2A-, receptor), CHRNA4 (cholinergic receptor, nicotinic, alpha 4), ADRB2 (adrenergic, beta-2-, receptor, surface), CCL11 (chemokine (C-C motif) ligand 11), GLUD1 (glutamate dehydrogenase 1), SLC6A1 (solute carrier family 6 (neurotransmitter transporter, GABA), member 1), CLDN5 (claudin 5), HDAC1 (histone deacetylase 1), HMGCR (3-hydroxy-3-methylglutaryl-Coenzyme A reductase), PSEN2 (presenilin 2 (Alzheimer disease 4)), ODC1 (ornithine decarboxylase 1), SELL (selectin L), PLG (plasminogen), HSPA8 (heat shock 70 kDa protein 8), TTR (transthyretin), GAST (gastrin), CYP17A1 (cytochrome P450, family 17, subfamily A, polypeptide 1), HRH1 (histamine receptor H1), ADM (adrenomedullin), GABRB2 (gamma-aminobutyric acid (GABA) A receptor, beta 2), NDE1 (nudE nuclear distribution gene E homolog 1 (A. nidulans)), TGFB3 (transforming growth factor, beta 3), XRCC1 (X-ray repair complementing defective repair in Chinese hamster cells 1), NPR1 (natriuretic peptide receptor A/guanylate cyclase A (atrionatriuretic peptide receptor A)), NGFR (nerve growth factor receptor (TNFR superfamily, member 16)), TUBB (tubulin, beta), G6PD (glucose-6-phosphate dehydrogenase), GSR (glutathione reductase), DDC (dopa decarboxylase (aromatic L-amino acid decarboxylase)), PON1 (paraoxonase 1), GABBR1 (gamma-aminobutyric acid (GABA) B receptor, 1), IGFBP1 (insulin-like growth factor binding protein 1), ALK (anaplastic lymphoma receptor tyrosine kinase), DHFR (dihydrofolate reductase), DPP4 (dipeptidyl-peptidase 4), ATF4 (activating transcription factor 4 (tax-responsive enhancer element B67)), GLS (glutaminase), NR3C2 (nuclear receptor subfamily 3, group C, member 2), ELANE (elastase, neutrophil expressed), MBL2 (mannose-binding lectin (protein C)2, soluble (opsonic defect)), HSPA1A (heat shock 70 kDa protein 1A), CYP3A5 (cytochrome P450, family 3, subfamily A, polypeptide 5), NOTCH2 (Notch homolog 2 (Drosophila)), TIMP3 (TIMP metallopeptidase inhibitor 3), GPX1 (glutathione peroxidase 1), TGM2 (transglutaminase 2 (C polypeptide, protein-glutamine-gamma-glutamyltransferase)), OPRM1 (opioid receptor, mu 1), UCP1 (uncoupling protein 1 (mitochondrial, proton carrier)), SCGB1A1 (secretoglobin, family 1A, member 1 (uteroglobin)), DAB1 (disabled homolog 1 (Drosophila)), NOD2 (nucleotide-binding oligomerization domain containing 2), ITGA4 (integrin, alpha 4 (antigen CD49D, alpha 4 subunit of VLA-4 receptor)), CPLX2 (complexin 2), MME (membrane metallo-endopeptidase), EMX2 (empty spiracles homeobox 2), HLA-C (major histocompatibility complex, class I, C), SLC1A1 (solute carrier family 1 (neuronal/epithelial high affinity glutamate transporter, system Xag), member 1), GHR (growth hormone receptor), GHRH (growth hormone releasing hormone), PCNT (pericentrin), ALDH2 (aldehyde dehydrogenase 2 family (mitochondrial)), IL4R (interleukin 4 receptor), SMARCA2 (SWI/SNF related, matrix associated, actin dependent regulator of chromatin, subfamily a, member 2), CDC42 (cell division cycle 42 (GTP binding protein, 25 kDa)), HLA-DPB1 (major histocompatibility complex, class II, DP beta 1), TKT (transketolase), TFAP2A (transcription factor AP-2 alpha (activating enhancer binding protein 2 alpha)), PIK3C2A (phosphoinositide-3-kinase, class 2, alpha polypeptide), MOG (myelin oligodendrocyte glycoprotein), GOT2 (glutamic-oxaloacetic transaminase 2, mitochondrial (aspartate aminotransferase 2)), KCNMA1 (potassium large conductance calcium-activated channel, subfamily M, alpha member 1), APOA5 (apolipoprotein A-V), RAP1A (RAP1A, member of RAS oncogene family), GABRG2 (gamma-aminobutyric acid (GABA) A receptor, gamma 2), FMR1 (fragile X mental retardation 1), AQP4 (aquaporin 4), FOXP2 (forkhead box P2), ADORA1 (adenosine A1 receptor), PLCG1 (phospholipase C, gamma 1), NRP1 (neuropilin 1), IFNA2 (interferon, alpha 2), CHGB (chromogranin B (secretogranin 1)), SERPINA1 (serpin peptidase inhibitor, Glade A (alpha-1 antiproteinase, antitrypsin), member 1), ARNT (aryl hydrocarbon receptor nuclear translocator), MLL (myeloid/lymphoid or mixed-lineage leukemia (trithorax homolog, Drosophila)), CXCL1 (chemokine (C-X-C motif) ligand 1 (melanoma growth stimulating activity, alpha)), TAP2 (transporter 2, ATP-binding cassette, sub-family B (MDR/TAP)), DDR1 (discoidin domain receptor tyrosine kinase 1), OXT (oxytocin, prepropeptide), L1CAM (L1 cell adhesion molecule), OPRK1 (opioid receptor, kappa 1), IL9 (interleukin 9), SRF (serum response factor (c-fos serum response element-binding transcription factor)), IL12B (interleukin 12B (natural killer cell stimulatory factor 2, cytotoxic lymphocyte maturation factor 2, p40)), GRP (gastrin-releasing peptide), NOTCH3 (Notch homolog 3 (Drosophila)), PRKDC (protein kinase, DNA-activated, catalytic polypeptide), MIF (macrophage migration inhibitory factor (glycosylation-inhibiting factor)), UCP2 (uncoupling protein 2 (mitochondrial, proton carrier)), CD9 (CD9 molecule), ATF2 (activating transcription factor 2), TSC2 (tuberous sclerosis 2), PAH (phenylalanine hydroxylase), TLR9 (toll-like receptor 9), PTH (parathyroid hormone), KCNH2 (potassium voltage-gated channel, subfamily H (eag-related), member 2), PLAUR (plasminogen activator, urokinase receptor), EGR2 (early growth response 2), LRP2 (low density lipoprotein receptor-related protein 2), CFDP1 (craniofacial development protein 1), ISL1 (ISL LIM homeobox 1), GABRA1 (gamma-aminobutyric acid (GABA) A receptor, alpha 1), HRH2 (histamine receptor H2), LIPC (lipase, hepatic), CACNA1A (calcium channel, voltage-dependent, P/Q type, alpha 1A subunit), GGT1 (gamma-glutamyltransferase 1), TPO (thyroid peroxidase), DNMT3A (DNA (cytosine-5-)-methyltransferase 3 alpha), NAT2 (N-acetyltransferase 2 (arylamine N-acetyltransferase)), FAAH (fatty acid amide hydrolase), PARK2 (Parkinson disease (autosomal recessive, juvenile)2, parkin), UCHL1 (ubiquitin carboxyl-terminal esterase L1 (ubiquitin thiolesterase)), KPNA3 (karyopherin alpha 3 (importin alpha 4)), HDAC4 (histone deacetylase 4), GJA5 (gap junction protein, alpha 5, 40 kDa), ALDH7A1 (aldehyde dehydrogenase 7 family, member A1), DMD (dystrophin), ITIH4 (inter-alpha (globulin) inhibitor H4 (plasma Kallikrein-sensitive glycoprotein)), HLA-G (major histocompatibility complex, class I, G), HTR4 (5-hydroxytryptamine (serotonin) receptor 4), ARSA (arylsulfatase A), WNT1 (wingless-type MMTV integration site family, member 1), MTR (5-methyltetrahydrofolate-homocysteine methyltransferase), TXN (thioredoxin), PAWR (PRKC, apoptosis, WT1, regulator), SRPX2 (sushi-repeat-containing protein, X-linked 2), NR4A1 (nuclear receptor subfamily 4, group A, member 1), EPRS (glutamyl-prolyl-tRNA synthetase), GRM8 (glutamate receptor, metabotropic 8), PPT1 (palmitoyl-protein thioesterase 1), LBR (lamin B receptor), SLC12A2 (solute carrier family 12 (sodium/potassium/chloride transporters), member 2), VLDLR (very low density lipoprotein receptor), PL-5283 (PL-5283 protein), CAMK2B (calcium/calmodulin-dependent protein kinase II beta), AGRN (agrin), CHI3L1 (chitinase 3-like 1 (cartilage glycoprotein-39)), PARK7 (Parkinson disease (autosomal recessive, early onset) 7), PINK1 (PTEN induced putative kinase 1), RXRB (retinoid X receptor, beta), GCLM (glutamate-cysteine ligase, modifier subunit), TBP (TATA box binding protein), MYLK (myosin light chain kinase), ARX (aristaless related homeobox), PLCB4 (phospholipase C, beta 4), NR2E1 (nuclear receptor subfamily 2, group E, member 1), GRM1 (glutamate receptor, metabotropic 1), CBS (cystathionine-beta-synthase), OPRD1 (opioid receptor, delta 1), PDE5A (phosphodiesterase 5A, cGMP-specific), ADRA1A (adrenergic, alpha-1A-, receptor), ATXN1 (ataxin 1), B3GAT1 (beta-1,3-glucuronyltransferase 1 (glucuronosyltransferase P)), PEBP1 (phosphatidylethanolamine binding protein 1), NF2 (neurofibromin 2 (merlin)), HSPA5 (heat shock 70 kDa protein 5 (glucose-regulated protein, 78 kDa)), PHB (prohibitin), SLC17A6 (solute carrier family 17 (sodium-dependent inorganic phosphate cotransporter), member 6), PPARD (peroxisome proliferator-activated receptor delta), BACE1 (beta-site APP-cleaving enzyme 1), TFAP2B (transcription factor AP-2 beta (activating enhancer binding protein 2 beta)), CDC25A (cell division cycle 25 homolog A (S. pombe)), LPA (lipoprotein, Lp(a)), APOA4 (apolipoprotein A-IV), PLA2G1 B (phospholipase A2, group IB (pancreas)), HCRT (hypocretin (orexin) neuropeptide precursor), PTGDS (prostaglandin D2 synthase 21 kDa (brain)), PPP3CA (protein phosphatase 3, catalytic subunit, alpha isozyme), CSNK2A1 (casein kinase 2, alpha 1 polypeptide), JUNB (jun B proto-oncogene), GTF3A (general transcription factor 111A), ATP2A2 (ATPase, Ca++ transporting, cardiac muscle, slow twitch 2), KCNQ2 (potassium voltage-gated channel, KQT-like subfamily, member 2), TAP1 (transporter 1, ATP-binding cassette, sub-family B (MDR/TAP)), PICK1 (protein interacting with PRKCA 1), SLC9A3R1 (solute carrier family 9 (sodium/hydrogen exchanger), member 3 regulator 1), SNCG (synuclein, gamma (breast cancer-specific protein 1)), RARG (retinoic acid receptor, gamma), SERPINA3 (serpin peptidase inhibitor, Glade A (alpha-1 antiproteinase, antitrypsin), member 3), GRIN2C (glutamate receptor, ionotropic, N-methyl D-aspartate 2C), GABRB3 (gamma-aminobutyric acid (GABA) A receptor, beta 3), NSF (N-ethylmaleimide-sensitive factor), GRIN2D (glutamate receptor, ionotropic, N-methyl D-aspartate 2D), HSPA1L (heat shock 70 kDa protein 1-like), TPH2 (tryptophan hydroxylase 2), MYH9 (myosin, heavy chain 9, non-muscle), GJD2 (gap junction protein, delta 2, 36 kDa), PDE3B (phosphodiesterase 3B, cGMP-inhibited), SMARCE1 (SWI/SNF related, matrix associated, actin dependent regulator of chromatin, subfamily e, member 1), LSAMP (limbic system-associated membrane protein), SLC2A3 (solute carrier family 2 (facilitated glucose transporter), member 3), LPAR1 (lysophosphatidic acid receptor 1), OGG1 (8-oxoguanine DNA glycosylase), CD59 (CD59 molecule, complement regulatory protein), SIM1 (single-minded homolog 1 (Drosophila)), RASA1 (RAS p21 protein activator (GTPase activating protein) 1), CALM1 (calmodulin 1 (phosphorylase kinase, delta)), CACNA1C (calcium channel, voltage-dependent, L type, alpha 1C subunit), ACLY (ATP citrate lyase), TUBA1B (tubulin, alpha 1b), ALDH1A1 (aldehyde dehydrogenase 1 family, member A1), GBA (glucosidase, beta, acid), PENK (proenkephalin), ABO (ABO blood group (transferase A, alpha 1-3-N-acetylgalactosaminyltransferase; transferase B, alpha 1-3-galactosyltransferase)), LIG4 (ligase IV, DNA, ATP-dependent), CA6 (carbonic anhydrase VI), CHRM2 (cholinergic receptor, muscarinic 2), SMPD1 (sphingomyelin phosphodiesterase 1, acid lysosomal), HSPA1 B (heat shock 70 kDa protein 1 B), HTR1D (5-hydroxytryptamine (serotonin) receptor 1 D), PAK3 (p21 protein (Cdc42/Rac)-activated kinase 3), SERPINI1 (serpin peptidase inhibitor, Glade I (neuroserpin), member 1), CAMK2A (calcium/calmodulin-dependent protein kinase II alpha), ROCK1 (Rho-associated, coiled-coil containing protein kinase 1), SET (SET nuclear oncogene), SREBF1 (sterol regulatory element binding transcription factor 1), GABRE (gamma-aminobutyric acid (GABA) A receptor, epsilon), OXTR (oxytocin receptor), STX1A (syntaxin 1A (brain)), TACR3 (tachykinin receptor 3), TUBA1A (tubulin, alpha 1a), CACNG2 (calcium channel, voltage-dependent, gamma subunit 2), ATN1 (atrophin 1), ADRBK2 (adrenergic, beta, receptor kinase 2), TNR (tenascin R (restrictin, janusin)), SPTA1 (spectrin, alpha, erythrocytic 1 (elliptocytosis 2)), PSENEN (presenilin enhancer 2 homolog (C. elegans)), MLXIPL (MLX interacting protein-like), P2RX7 (purinergic receptor P2X, ligand-gated ion channel, 7), GSK3A (glycogen synthase kinase 3 alpha), IMPA1 (inositol(myo)-1(or 4)-monophosphatase 1), GADD45A (growth arrest and DNA-damage-inducible, alpha), PPARGC1A (peroxisome proliferator-activated receptor gamma, coactivator 1 alpha), PDE4A (phosphodiesterase 4A, cAMP-specific (phosphodiesterase E2 dunce homolog, Drosophila)), SGCB (sarcoglycan, beta (43 kDa dystrophin-associated glycoprotein)), HLA-DRB5 (major histocompatibility complex, class II, DR beta 5), SIRPA (signal-regulatory protein alpha), PPP2R4 (protein phosphatase 2A activator, regulatory subunit 4), ENPP1 (ectonucleotide pyrophosphatase/phosphodiesterase 1), ANK3 (ankyrin 3, node of Ranvier (ankyrin G)), GHRL (ghrelin/obestatin prepropeptide), RCAN1 (regulator of calcineurin 1), ABAT (4-aminobutyrate aminotransferase), VAMP2 (vesicle-associated membrane protein 2 (synaptobrevin 2)), EXT1 (exostoses (multiple) 1), TCF3 (transcription factor 3 (E2A immunoglobulin enhancer binding factors E12/E47)), WFS1 (Wolfram syndrome 1 (wolframin)), BBS4 (Bardet-Biedl syndrome 4), FRZB (frizzled-related protein), TRH (thyrotropin-releasing hormone), SLC17A5 (solute carrier family 17 (anion/sugar transporter), member 5), CLOCK (clock homolog (mouse)), GABRB1 (gamma-aminobutyric acid (GABA) A receptor, beta 1), RGS2 (regulator of G-protein signaling 2, 24 kDa), NTSR1 (neurotensin receptor 1 (high affinity)), DRP2 (dystrophin related protein 2), SLC18A1 (solute carrier family 18 (vesicular monoamine), member 1), PCM1 (pericentriolar material 1), SLC1A6 (solute carrier family 1 (high affinity aspartate/glutamate transporter), member 6), SNAR-E (small ILF3/NF90-associated RNA E), ADAM12 (ADAM metallopeptidase domain 12), IL9R (interleukin 9 receptor), GSN (gelsolin), GC (group-specific component (vitamin D binding protein)), HDAC3 (histone deacetylase 3), KCNE1 (potassium voltage-gated channel, Isk-related family, member 1), SLC12A6 (solute carrier family 12 (potassium/chloride transporters), member 6), ABCC4 (ATP-binding cassette, sub-family C(CFTR/MRP), member 4), NUDT6 (nudix (nucleoside diphosphate linked moiety X)-type motif 6), SLC22A4 (solute carrier family 22 (organic cation/ergothioneine transporter), member 4), FLNA (filamin A, alpha), COL7A1 (collagen, type VII, alpha 1), PAM (peptidylglycine alpha-amidating monooxygenase), ADRA2C (adrenergic, alpha-2C—, receptor), FOSB (FBJ murine osteosarcoma viral oncogene homolog B), GABRA5 (gamma-aminobutyric acid (GABA) A receptor, alpha 5), VCP (valosin-containing protein), BSG (basigin (Ok blood group)), CRY1 (cryptochrome 1 (photolyase-like)), HNMT (histamine N-methyltransferase), HCRTR1 (hypocretin (orexin) receptor 1), CFL1 (cofilin 1 (non-muscle)), FBP1 (fructose-1,6-bisphosphatase 1), ESD (esterase D/formylglutathione hydrolase), DCTN2 (dynactin 2 (p50)), DYNC1H1 (dynein, cytoplasmic 1, heavy chain 1), CRYAB (crystallin, alpha B), CASK (calcium/calmodulin-dependent serine protein kinase (MAGUK family)), SMAD5 (SMAD family member 5), SMARCC1 (SWI/SNF related, matrix associated, actin dependent regulator of chromatin, subfamily c, member 1), UTRN (utrophin), KCNE2 (potassium voltage-gated channel, Isk-related family, member 2), HLA-E (major histocompatibility complex, class I, E), TPI1 (triosephosphate isomerase 1), CACNA1B (calcium channel, voltage-dependent, N type, alpha 1B subunit), SCD (stearoyl-CoA desaturase (delta-9-desaturase)), HLA-DPA1 (major histocompatibility complex, class II, DP alpha 1), PADI4 (peptidyl arginine deiminase, type IV), HLA-DRB3 (major histocompatibility complex, class II, DR beta 3), HLA-DRA (major histocompatibility complex, class II, DR alpha), YY1 (YY1 transcription factor), SYNJ1(synaptojanin 1), COL4A1 (collagen, type IV, alpha 1), AXIN1 (axin 1), MC2R (melanocortin 2 receptor (adrenocorticotropic hormone)), RAI1 (retinoic acid induced 1), TARDBP (TAR DNA binding protein), RLN1 (relaxin 1), PXK (PX domain containing serine/threonine kinase), PDE4D (phosphodiesterase 4D, cAMP-specific (phosphodiesterase E3 dunce homolog, Drosophila)), CD99 (CD99 molecule), MAT1A (methionine adenosyltransferase I, alpha), ARNTL (aryl hydrocarbon receptor nuclear translocator-like), SLC25A3 (solute carrier family 25 (mitochondrial carrier; phosphate carrier), member 3), IMMT (inner membrane protein, mitochondrial (mitofilin)), EARS2 (glutamyl-tRNA synthetase 2, mitochondrial (putative)), BSN (bassoon (presynaptic cytomatrix protein)), NALCN (sodium leak channel, non-selective), CNTLN (centlein, centrosomal protein), MST1 R (macrophage stimulating 1 receptor (c-met-related tyrosine kinase)), AVPR1A (arginine vasopressin receptor 1A), HSP90B1 (heat shock protein 90 kDa beta (Grp94), member 1), ATP2B2 (ATPase, Ca++ transporting, plasma membrane 2), GRK6 (G protein-coupled receptor kinase 6), CYSLTR2 (cysteinyl leukotriene receptor 2), 5-Sep(septin 5), FADS2 (fatty acid desaturase 2), SLC18A3 (solute carrier family 18 (vesicular acetylcholine), member 3), PPIA (peptidylprolyl isomerase A (cyclophilin A)), HNRNPK (heterogeneous nuclear ribonucleoprotein K), PER1 (period homolog 1 (Drosophila)), IDE (insulin-degrading enzyme), CSNK1E (casein kinase 1, epsilon), TRAF1 (TNF receptor-associated factor 1), STUB1 (STIP1 homology and U-box containing protein 1), BIRC2 (baculoviral IAP repeat-containing 2), OMG (oligodendrocyte myelin glycoprotein), MTRR (5-methyltetrahydrofolate-homocysteine methyltransferase reductase), NPAS2 (neuronal PAS domain protein 2), PCMT1 (protein-L-isoaspartate (D-aspartate) O-methyltransferase), FABP5 (fatty acid binding protein 5 (psoriasis-associated)), GGT2 (gamma-glutamyltransferase 2), ACTN2 (actinin, alpha 2), GABRA2 (gamma-aminobutyric acid (GABA) A receptor, alpha 2), TGFBI (transforming growth factor, beta-induced, 68 kDa), TPT1 (tumor protein, translationally-controlled 1), FTL (ferritin, light polypeptide), AP3B1 (adaptor-related protein complex 3, beta 1 subunit), PTAFR (platelet-activating factor receptor), PER2 (period homolog 2 (Drosophila)), NPEPPS (aminopeptidase puromycin sensitive), PIP5K1B (phosphatidylinositol-4-phosphate 5-kinase, type I, beta), DLAT (dihydrolipoamide S-acetyltransferase), PALLD (palladin, cytoskeletal associated protein), ERBB21P (erbb2 interacting protein), STX7 (syntaxin 7), FADS1 (fatty acid desaturase 1), LIPF (lipase, gastric), TIAL1 (TIA1 cytotoxic granule-associated RNA binding protein-like 1), DNAJC3 (DnaJ (Hsp40) homolog, subfamily C, member 3), FCRL3 (Fc receptor-like 3), UGT1A4 (UDP glucuronosyltransferase 1 family, polypeptide A4), and MT-ND1 (mitochondrially encoded NADH dehydrogenase 1).

In yet another iteration of the disclosure, suitable chromosomal sequences associated with schizophrenia and combinations of these chromosomal sequences are detailed in Table A. For example, those rows having no entry in the “Protein Sequence” column indicate a genetically modified animal in which the sequence specified in that row under “Activated Sequence” is inactivated (i.e., a knock-out). Subsequent rows indicate single or multiple knock-outs with knock-ins of one or more integrated orthologous sequences, as indicated in the “Protein Sequence” column.

TABLE A

| Activated Sequence | Protein Sequence |
|---|---|
| nrg1 | none |
| erbb4 | none |
| cplx1 | none |
| tph1 | none |
| tph2 | none |
| gsk3a | none |
| gsk3b | none |
| nrxn1 | none |
| bdnf | none |
| disc1 | none |
| nrg1, erbb4 | NRG1, ErbB4 |
| nrg1, cplx1 | NRG1, CPLX1 |
| nrg1, tph1 | NRG1, TPH1 |
| nrg1, tph2 | NRG1, TPH2 |
| nrg1, gsk3a | NRG1, GSK3A |
| nrg1, gsk3b | NRG1, GSK3B |
| nrg1, nrxn1 | NRG1, NRXN1 |
| nrg1, bdnf | NRG1, BDNF |
| nrg1, disc1 | NRG1, DISC1 |
| erbb4, cplx1 | ErbB4, CPLX1 |
| erbb4, tph1 | ErbB4, TPH1 |
| erbb4, tph2 | ErbB4, TPH2 |
| erbb4, gsk3a | ErbB4, GSK3A |
| erbb4, gsk3b | ErbB4, GSK3B |
| erbb4, nrxn1 | ErbB4, NRXN1 |
| erbb4, bdnf | ErbB4, BDNF |
| erbb4, disc1 | ErbB4, DISC1 |
| cplx1, tph1 | CPLX1, TPH1 |
| cplx1, tph2 | CPLX1, TPH2 |
| cplx1, gsk3a | CPLX1, GSK3A |
| cplx1, gsk3b | CPLX1, GSK3B |
| cplx1, nrxn1 | CPLX1, NRXN1 |
| cplx1, bdnf | CPLX1, BDNF |
| cplx1, disc1 | CPLX1, DISC1 |
| tph1, tph2 | TPH1, TPH2 |
| tph1, gsk3a | TPH1, GSK3A |
| tph1, gsk3b | TPH1, GSK3B |
| tph1, nrxn1 | TPH1, NRXN1 |
| tph1, bdnf | TPH1, BDNF |
| tph1, disc1 | TPH1, DISC1 |
| tph2, gsk3a | TPH2, GSK3A |
| tph2, gsk3b | TPH2, GSK3B |
| tph2, nrxn1 | TPH2, NRXN1 |
| tph2, bdnf | TPH2, BDNF |
| tph2, disc1 | TPH2, DISC1 |
| gsk3a, gsk3b | GSK3A, GSK3B |
| gsk3a, nrxn1 | GSK3A, NRXN1 |
| gsk3a, bdnf | GSK3A, BDNF |
| gsk3a, disc1 | GSK3A, DISC1 |
| gsk3b, nrxn1 | GSK3B, NRXN1 |
| gsk3b, bdnf | GSK3B, BDNF |
| gsk3b, disc1 | GSK3B, DISC1 |
| nrxn1, bdnf | NRXN1, BDNF |
| nrxn1, disc1 | NRXN1, DISC1 |
| bdnf, disc1 | BDNF, DISC1 |
| nrg1, erbb4, cplx1 | NRG1, ErbB4, CPLX1 |
| nrg1, erbb4, tph1 | NRG1, ErbB4, TPH1 |
| nrg1, erbb4, tph2 | NRG1, ErbB4, TPH2 |
| nrg1, erbb4, gsk3a | NRG1, ErbB4, GSK3A |
| nrg1, erbb4, gsk3b | NRG1, ErbB4, GSK3B |
| nrg1, erbb4, nrxn1 | NRG1, ErbB4, NRXN1 |
| nrg1, erbb4, bdnf | NRG1, ErbB4, BDNF |
| nrg1, erbb4, disc1 | NRG1, ErbB4, DISC1 |
| nrg1, cplx1, tph1 | NRG1, CPLX1, TPH1 |
| nrg1, cplx1, tph2 | NRG1, CPLX1, TPH2 |
| nrg1, cplx1, gsk3a | NRG1, CPLX1, GSK3A |
| nrg1, cplx1, gsk3b | NRG1, CPLX1, GSK3B |
| nrg1, cplx1, nrxn1 | NRG1, CPLX1, NRXN1 |
| nrg1, cplx1, bdnf | NRG1, CPLX1, BDNF |
| nrg1, cplx1, disc1 | NRG1, CPLX1, DISC1 |
| nrg1, tph1, tph2 | NRG1, TPH1, TPH2 |
| nrg1, tph1, gsk3a | NRG1, TPH1, GSK3A |
| nrg1, tph1, gsk3b | NRG1, TPH1, GSK3B |
| nrg1, tph1, nrxn1 | NRG1, TPH1, NRXN1 |
| nrg1, tph1, bdnf | NRG1, TPH1, BDNF |
| nrg1, tph1, disc1 | NRG1, TPH1, DISC1 |
| nrg1, tph2, gsk3a | NRG1, TPH2, GSK3A |
| nrg1, tph2, gsk3b | NRG1, TPH2, GSK3B |
| nrg1, tph2, nrxn1 | NRG1, TPH2, NRXN1 |
| nrg1, tph2, bdnf | NRG1, TPH2, BDNF |
| nrg1, tph2, disc1 | NRG1, TPH2, DISC1 |
| nrg1, gsk3a, gsk3b | NRG1, GSK3A, GSK3B |
| nrg1, gsk3a, nrxn1 | NRG1, GSK3A, NRXN1 |
| nrg1, gsk3a, bdnf | NRG1, GSK3A, BDNF |
| nrg1, gsk3a, disc1 | NRG1, GSK3A, DISC1 |
| nrg1, gsk3b, nrxn1 | NRG1, GSK3B, NRXN1 |
| nrg1, gsk3b, bdnf | NRG1, GSK3B, BDNF |
| nrg1, gsk3b, disc1 | NRG1, GSK3B, DISC1 |
| nrg1, nrxn1, bdnf | NRG1, NRXN1, BDNF |
| nrg1, nrxn1, disc1 | NRG1, NRXN1, DISC1 |
| nrg1, bdnf, disc1 | NRG1, BDNF, DISC1 |
| erbb4, cplx1, tph1 | ErbB4, CPLX1, TPH1 |
| erbb4, cplx1, tph2 | ErbB4, CPLX1, TPH2 |
| erbb4, cplx1, gsk3a | ErbB4, CPLX1, GSK3A |
| erbb4, cplx1, gsk3b | ErbB4, CPLX1, GSK3B |
| erbb4, cplx1, nrxn1 | ErbB4, CPLX1, NRXN1 |
| erbb4, cplx1, bdnf | ErbB4, CPLX1, BDNF |
| erbb4, cplx1, disc1 | ErbB4, CPLX1, DISC1 |
| erbb4, tph1, tph2 | ErbB4, TPH1, TPH2 |
| erbb4, tph1, gsk3a | ErbB4, TPH1, GSK3A |
| erbb4, tph1, gsk3b | ErbB4, TPH1, GSK3B |
| erbb4, tph1, nrxn1 | ErbB4, TPH1, NRXN1 |
| erbb4, tph1, bdnf | ErbB4, TPH1, BDNF |
| erbb4, tph1, disc1 | ErbB4, TPH1, DISC1 |
| erbb4, tph2, gsk3a | ErbB4, TPH2, GSK3A |
| erbb4, tph2, gsk3b | ErbB4, TPH2, GSK3B |
| erbb4, tph2, nrxn1 | ErbB4, TPH2, NRXN1 |
| erbb4, tph2, bdnf | ErbB4, TPH2, BDNF |
| erbb4, tph2, disc1 | ErbB4, TPH2, DISC1 |
| erbb4, gsk3a, gsk3b | ErbB4, GSK3A, GSK3B |
| erbb4, gsk3a, nrxn1 | ErbB4, GSK3A, NRXN1 |
| erbb4, gsk3a, bdnf | ErbB4, GSK3A, BDNF |
| erbb4, gsk3a, disc1 | ErbB4, GSK3A, DISC1 |
| erbb4, gsk3b, nrxn1 | ErbB4, GSK3B, NRXN1 |
| erbb4, gsk3b, bdnf | ErbB4, GSK3B, BDNF |
| erbb4, gsk3b, disc1 | ErbB4, GSK3B, DISC1 |
| erbb4, nrxn1, bdnf | ErbB4, NRXN1, BDNF |
| erbb4, nrxn1, disc1 | ErbB4, NRXN1, DISC1 |
| erbb4, bdnf, disc1 | ErbB4, BDNF, DISC1 |
| cplx1, tph1, tph2 | CPLX1, TPH1, TPH2 |
| cplx1, tph1, gsk3a | CPLX1, TPH1, GSK3A |
| cplx1, tph1, gsk3b | CPLX1, TPH1, GSK3B |
| cplx1, tph1, nrxn1 | CPLX1, TPH1, NRXN1 |
| cplx1, tph1, bdnf | CPLX1, TPH1, BDNF |
| cplx1, tph1, disc1 | CPLX1, TPH1, DISC1 |
| cplx1, tph2, gsk3a | CPLX1, TPH2, GSK3A |
| cplx1, tph2, gsk3b | CPLX1, TPH2, GSK3B |
| cplx1, tph2, nrxn1 | CPLX1, TPH2, NRXN1 |
| cplx1, tph2, bdnf | CPLX1, TPH2, BDNF |
| cplx1, tph2, disc1 | CPLX1, TPH2, DISC1 |
| cplx1, gsk3a, gsk3b | CPLX1, GSK3A, GSK3B |
| cplx1, gsk3a, nrxn1 | CPLX1, GSK3A, NRXN1 |
| cplx1, gsk3a, bdnf | CPLX1, GSK3A, BDNF |
| cplx1, gsk3a, disc1 | CPLX1, GSK3A, DISC1 |
| cplx1, gsk3b, nrxn1 | CPLX1, GSK3B, NRXN1 |
| cplx1, gsk3b, bdnf | CPLX1, GSK3B, BDNF |
| cplx1, gsk3b, disc1 | CPLX1, GSK3B, DISC1 |
| cplx1, nrxn1, bdnf | CPLX1, NRXN1, BDNF |
| cplx1, nrxn1, disc1 | CPLX1, NRXN1, DISC1 |
| cplx1, bdnf, disc1 | CPLX1, BDNF, DISC1 |
| tph1, tph2, gsk3a | TPH1, TPH2, GSK3A |
| tph1, tph2, gsk3b | TPH1, TPH2, GSK3B |
| tph1, tph2, nrxn1 | TPH1, TPH2, NRXN1 |
| tph1, tph2, bdnf | TPH1, TPH2, BDNF |
| tph1, tph2, disc1 | TPH1, TPH2, DISC1 |
| tph1, gsk3a, gsk3b | TPH1, GSK3A, GSK3B |
| tph1, gsk3a, nrxn1 | TPH1, GSK3A, NRXN1 |
| tph1, gsk3a, bdnf | TPH1, GSK3A, BDNF |
| tph1, gsk3a, disc1 | TPH1, GSK3A, DISC1 |
| tph1, gsk3b, nrxn1 | TPH1, GSK3B, NRXN1 |
| tph1, gsk3b, bdnf | TPH1, GSK3B, BDNF |
| tph1, gsk3b, disc1 | TPH1, GSK3B, DISC1 |
| tph1, nrxn1, bdnf | TPH1, NRXN1, BDNF |
| tph1, nrxn1, disc1 | TPH1, NRXN1, DISC1 |
| tph1, bdnf, disc1 | TPH1, BDNF, DISC1 |
| tph2, gsk3a, gsk3b | TPH2, GSK3A, GSK3B |
| tph2, gsk3a, nrxn1 | TPH2, GSK3A, NRXN1 |
| tph2, gsk3a, bdnf | TPH2, GSK3A, BDNF |
| tph2, gsk3a, disc1 | TPH2, GSK3A, DISC1 |
| tph2, gsk3b, nrxn1 | TPH2, GSK3B, NRXN1 |
| tph2, gsk3b, bdnf | TPH2, GSK3B, BDNF |
| tph2, gsk3b, disc1 | TPH2, GSK3B, DISC1 |
| tph2, nrxn1, bdnf | TPH2, NRXN1, BDNF |
| tph2, nrxn1, disc1 | TPH2, NRXN1, DISC1 |
| tph2, bdnf, disc1 | TPH2, BDNF, DISC1 |
| gsk3a, gsk3b, nrxn1 | GSK3A, GSK3B, NRXN1 |
| gsk3a, gsk3b, bdnf | GSK3A, GSK3B, BDNF |
| gsk3a, gsk3b, disc1 | GSK3A, GSK3B, DISC1 |
| gsk3a, nrxn1, bdnf | GSK3A, NRXN1, BDNF |
| gsk3a, nrxn1, disc1 | GSK3A, NRXN1, DISC1 |
| gsk3a, bdnf, disc1 | GSK3A, BDNF, DISC1 |
| gsk3b, nrxn1, bdnf | GSK3B, NRXN1, BDNF |
| gsk3b, nrxn1, disc1 | GSK3B, NRXN1, DISC1 |
| gsk3b, bdnf, disc1 | GSK3B, BDNF, DISC1 |
| nrxn1, bdnf, disc1 | NRXN1, BDNF, DISC1 |
| nrg1, erbb4, cplx1, tph1 | NRG1, ErbB4, CPLX1, TPH1 |
| nrg1, erbb4, cplx1, tph2 | NRG1, ErbB4, CPLX1, TPH2 |
| nrg1, erbb4, cplx1, gsk3a | NRG1, ErbB4, CPLX1, GSK3A |
| nrg1, erbb4, cplx1, gsk3b | NRG1, ErbB4, CPLX1, GSK3B |
| nrg1, erbb4, cplx1, nrxn1 | NRG1, ErbB4, CPLX1, NRXN1 |
| nrg1, erbb4, cplx1, bdnf | NRG1, ErbB4, CPLX1, BDNF |
| nrg1, erbb4, cplx1, disc1 | NRG1, ErbB4, CPLX1, DISC1 |
| nrg1, erbb4, tph1, tph2 | NRG1, ErbB4, TPH1, TPH2 |
| nrg1, erbb4, tph1, gsk3a | NRG1, ErbB4, TPH1, GSK3A |
| nrg1, erbb4, tph1, gsk3b | NRG1, ErbB4, TPH1, GSK3B |
| nrg1, erbb4, tph1, nrxn1 | NRG1, ErbB4, TPH1, NRXN1 |
| nrg1, erbb4, tph1, bdnf | NRG1, ErbB4, TPH1, BDNF |
| nrg1, erbb4, tph1, disc1 | NRG1, ErbB4, TPH1, DISC1 |
| nrg1, erbb4, tph2, gsk3a | NRG1, ErbB4, TPH2, GSK3A |
| nrg1, erbb4, tph2, gsk3b | NRG1, ErbB4, TPH2, GSK3B |
| nrg1, erbb4, tph2, nrxn1 | NRG1, ErbB4, TPH2, NRXN1 |
| nrg1, erbb4, tph2, bdnf | NRG1, ErbB4, TPH2, BDNF |
| nrg1, erbb4, tph2, disc1 | NRG1, ErbB4, TPH2, DISC1 |
| nrg1, erbb4, gsk3a, gsk3b | NRG1, ErbB4, GSK3A, GSK3B |
| nrg1, erbb4, gsk3a, nrxn1 | NRG1, ErbB4, GSK3A, NRXN1 |
| nrg1, erbb4, gsk3a, bdnf | NRG1, ErbB4, GSK3A, BDNF |
| nrg1, erbb4, gsk3a, disc1 | NRG1, ErbB4, GSK3A, DISC1 |
| nrg1, erbb4, gsk3b, nrxn1 | NRG1, ErbB4, GSK3B, NRXN1 |
| nrg1, erbb4, gsk3b, bdnf | NRG1, ErbB4, GSK3B, BDNF |
| nrg1, erbb4, gsk3b, disc1 | NRG1, ErbB4, GSK3B, DISC1 |
| nrg1, erbb4, nrxn1, bdnf | NRG1, ErbB4, NRXN1, BDNF |
| nrg1, erbb4, nrxn1, disc1 | NRG1, ErbB4, NRXN1, DISC1 |
| nrg1, erbb4, bdnf, disc1 | NRG1, ErbB4, BDNF, DISC1 |
| nrg1, cplx1, tph1, tph2 | NRG1, CPLX1, TPH1, TPH2 |
| nrg1, cplx1, tph1, gsk3a | NRG1, CPLX1, TPH1, GSK3A |
| nrg1, cplx1, tph1, gsk3b | NRG1, CPLX1, TPH1, GSK3B |
| nrg1, cplx1, tph1, nrxn1 | NRG1, CPLX1, TPH1, NRXN1 |
| nrg1, cplx1, tph1, bdnf | NRG1, CPLX1, TPH1, BDNF |
| nrg1, cplx1, tph1, disc1 | NRG1, CPLX1, TPH1, DISC1 |
| nrg1, cplx1, tph2, gsk3a | NRG1, CPLX1, TPH2, GSK3A |
| nrg1, cplx1, tph2, gsk3b | NRG1, CPLX1, TPH2, GSK3B |
| nrg1, cplx1, tph2, nrxn1 | NRG1, CPLX1, TPH2, NRXN1 |
| nrg1, cplx1, tph2, bdnf | NRG1, CPLX1, TPH2, BDNF |
| nrg1, cplx1, tph2, disc1 | NRG1, CPLX1, TPH2, DISC1 |
| nrg1, cplx1, gsk3a, gsk3b | NRG1, CPLX1, GSK3A, GSK3B |
| nrg1, cplx1, gsk3a, nrxn1 | NRG1, CPLX1, GSK3A, NRXN1 |
| nrg1, cplx1, gsk3a, bdnf | NRG1, CPLX1, GSK3A, BDNF |
| nrg1, cplx1, gsk3a, disc1 | NRG1, CPLX1, GSK3A, DISC1 |
| nrg1, cplx1, gsk3b, nrxn1 | NRG1, CPLX1, GSK3B, NRXN1 |
| nrg1, cplx1, gsk3b, bdnf | NRG1, CPLX1, GSK3B, BDNF |
| nrg1, cplx1, gsk3b, disc1 | NRG1, CPLX1, GSK3B, DISC1 |
| nrg1, cplx1, nrxn1, bdnf | NRG1, CPLX1, NRXN1, BDNF |
| nrg1, cplx1, nrxn1, disc1 | NRG1, CPLX1, NRXN1, DISC1 |
| nrg1, cplx1, bdnf, disc1 | NRG1, CPLX1, BDNF, DISC1 |
| nrg1, tph1, tph2, gsk3a | NRG1, TPH1, TPH2, GSK3A |
| nrg1, tph1, tph2, gsk3b | NRG1, TPH1, TPH2, GSK3B |
| nrg1, tph1, tph2, nrxn1 | NRG1, TPH1, TPH2, NRXN1 |
| nrg1, tph1, tph2, bdnf | NRG1, TPH1, TPH2, BDNF |
| nrg1, tph1, tph2, disc1 | NRG1, TPH1, TPH2, DISC1 |
| nrg1, tph1, gsk3a, gsk3b | NRG1, TPH1, GSK3A, GSK3B |
| nrg1, tph1, gsk3a, nrxn1 | NRG1, TPH1, GSK3A, NRXN1 |
| nrg1, tph1, gsk3a, bdnf | NRG1, TPH1, GSK3A, BDNF |
| nrg1, tph1, gsk3a, disc1 | NRG1, TPH1, GSK3A, DISC1 |
| nrg1, tph1, gsk3b, nrxn1 | NRG1, TPH1, GSK3B, NRXN1 |
| nrg1, tph1, gsk3b, bdnf | NRG1, TPH1, GSK3B, BDNF |
| nrg1, tph1, gsk3b, disc1 | NRG1, TPH1, GSK3B, DISC1 |
| nrg1, tph1, nrxn1, bdnf | NRG1, TPH1, NRXN1, BDNF |
| nrg1, tph1, nrxn1, disc1 | NRG1, TPH1, NRXN1, DISC1 |
| nrg1, tph1, bdnf, disc1 | NRG1, TPH1, BDNF, DISC1 |
| nrg1, tph2, gsk3a, gsk3b | NRG1, TPH2, GSK3A, GSK3B |
| nrg1, tph2, gsk3a, nrxn1 | NRG1, TPH2, GSK3A, NRXN1 |
| nrg1, tph2, gsk3a, bdnf | NRG1, TPH2, GSK3A, BDNF |
| nrg1, tph2, gsk3a, disc1 | NRG1, TPH2, GSK3A, DISC1 |
| nrg1, tph2, gsk3b, nrxn1 | NRG1, TPH2, GSK3B, NRXN1 |
| nrg1, tph2, gsk3b, bdnf | NRG1, TPH2, GSK3B, BDNF |
| nrg1, tph2, gsk3b, disc1 | NRG1, TPH2, GSK3B, DISC1 |
| nrg1, tph2, nrxn1, bdnf | NRG1, TPH2, NRXN1, BDNF |
| nrg1, tph2, nrxn1, disc1 | NRG1, TPH2, NRXN1, DISC1 |
| nrg1, tph2, bdnf, disc1 | NRG1, TPH2, BDNF, DISC1 |
| nrg1, gsk3a, gsk3b, nrxn1 | NRG1, GSK3A, GSK3B, NRXN1 |
| nrg1, gsk3a, gsk3b, bdnf | NRG1, GSK3A, GSK3B, BDNF |
| nrg1, gsk3a, gsk3b, disc1 | NRG1, GSK3A, GSK3B, DISC1 |
| nrg1, gsk3a, nrxn1, bdnf | NRG1, GSK3A, NRXN1, BDNF |
| nrg1, gsk3a, nrxn1, disc1 | NRG1, GSK3A, NRXN1, DISC1 |
| nrg1, gsk3a, bdnf, disc1 | NRG1, GSK3A, BDNF, DISC1 |
| nrg1, gsk3b, nrxn1, bdnf | NRG1, GSK3B, NRXN1, BDNF |
| nrg1, gsk3b, nrxn1, disc1 | NRG1, GSK3B, NRXN1, DISC1 |
| nrg1, gsk3b, bdnf, disc1 | NRG1, GSK3B, BDNF, DISC1 |
| nrg1, nrxn1, bdnf, disc1 | NRG1, NRXN1, BDNF, DISC1 |
| erbb4, cplx1, tph1, tph2 | ErbB4, CPLX1, TPH1, TPH2 |
| erbb4, cplx1, tph1, gsk3a | ErbB4, CPLX1, TPH1, GSK3A |
| erbb4, cplx1, tph1, gsk3b | ErbB4, CPLX1, TPH1, GSK3B |
| erbb4, cplx1, tph1, nrxn1 | ErbB4, CPLX1, TPH1, NRXN1 |
| erbb4, cplx1, tph1, bdnf | ErbB4, CPLX1, TPH1, BDNF |
| erbb4, cplx1, tph1, disc1 | ErbB4, CPLX1, TPH1, DISC1 |
| erbb4, cplx1, tph2, gsk3a | ErbB4, CPLX1, TPH2, GSK3A |
| erbb4, cplx1, tph2, gsk3b | ErbB4, CPLX1, TPH2, GSK3B |
| erbb4, cplx1, tph2, nrxn1 | ErbB4, CPLX1, TPH2, NRXN1 |
| erbb4, cplx1, tph2, bdnf | ErbB4, CPLX1, TPH2, BDNF |
| erbb4, cplx1, tph2, disc1 | ErbB4, CPLX1, TPH2, DISC1 |
| erbb4, cplx1, gsk3a, gsk3b | ErbB4, CPLX1, GSK3A, GSK3B |
| erbb4, cplx1, gsk3a, nrxn1 | ErbB4, CPLX1, GSK3A, NRXN1 |
| erbb4, cplx1, gsk3a, bdnf | ErbB4, CPLX1, GSK3A, BDNF |
| erbb4, cplx1, gsk3a, disc1 | ErbB4, CPLX1, GSK3A, DISC1 |
| erbb4, cplx1, gsk3b, nrxn1 | ErbB4, CPLX1, GSK3B, NRXN1 |
| erbb4, cplx1, gsk3b, bdnf | ErbB4, CPLX1, GSK3B, BDNF |
| erbb4, cplx1, gsk3b, disc1 | ErbB4, CPLX1, GSK3B, DISC1 |
| erbb4, cplx1, nrxn1, bdnf | ErbB4, CPLX1, NRXN1, BDNF |
| erbb4, cplx1, nrxn1, disc1 | ErbB4, CPLX1, NRXN1, DISC1 |
| erbb4, cplx1, bdnf, disc1 | ErbB4, CPLX1, BDNF, DISC1 |
| erbb4, tph1, tph2, gsk3a | ErbB4, TPH1, TPH2, GSK3A |
| erbb4, tph1, tph2, gsk3b | ErbB4, TPH1, TPH2, GSK3B |
| erbb4, tph1, tph2, nrxn1 | ErbB4, TPH1, TPH2, NRXN1 |
| erbb4, tph1, tph2, bdnf | ErbB4, TPH1, TPH2, BDNF |
| erbb4, tph1, tph2, disc1 | ErbB4, TPH1, TPH2, DISC1 |
| erbb4, tph1, gsk3a, gsk3b | ErbB4, TPH1, GSK3A, GSK3B |
| erbb4, tph1, gsk3a, nrxn1 | ErbB4, TPH1, GSK3A, NRXN1 |
| erbb4, tph1, gsk3a, bdnf | ErbB4, TPH1, GSK3A, BDNF |
| erbb4, tph1, gsk3a, disc1 | ErbB4, TPH1, GSK3A, DISC1 |
| erbb4, tph1, gsk3b, nrxn1 | ErbB4, TPH1, GSK3B, NRXN1 |
| erbb4, tph1, gsk3b, bdnf | ErbB4, TPH1, GSK3B, BDNF |
| erbb4, tph1, gsk3b, disc1 | ErbB4, TPH1, GSK3B, DISC1 |
| erbb4, tph1, nrxn1, bdnf | ErbB4, TPH1, NRXN1, BDNF |
| erbb4, tph1, nrxn1, disc1 | ErbB4, TPH1, NRXN1, DISC1 |
| erbb4, tph1, bdnf, disc1 | ErbB4, TPH1, BDNF, DISC1 |
| erbb4, tph2, gsk3a, gsk3b | ErbB4, TPH2, GSK3A, GSK3B |
| erbb4, tph2, gsk3a, nrxn1 | ErbB4, TPH2, GSK3A, NRXN1 |
| erbb4, tph2, gsk3a, bdnf | ErbB4, TPH2, GSK3A, BDNF |
| erbb4, tph2, gsk3a, disc1 | ErbB4, TPH2, GSK3A, DISC1 |
| erbb4, tph2, gsk3b, nrxn1 | ErbB4, TPH2, GSK3B, NRXN1 |
| erbb4, tph2, gsk3b, bdnf | ErbB4, TPH2, GSK3B, BDNF |
| erbb4, tph2, gsk3b, disc1 | ErbB4, TPH2, GSK3B, DISC1 |
| erbb4, tph2, nrxn1, bdnf | ErbB4, TPH2, NRXN1, BDNF |
| erbb4, tph2, nrxn1, disc1 | ErbB4, TPH2, NRXN1, DISC1 |
| erbb4, tph2, bdnf, disc1 | ErbB4, TPH2, BDNF, DISC1 |
| erbb4, gsk3a, gsk3b, nrxn1 | ErbB4, GSK3A, GSK3B, NRXN1 |
| erbb4, gsk3a, gsk3b, bdnf | ErbB4, GSK3A, GSK3B, BDNF |
| erbb4, gsk3a, gsk3b, disc1 | ErbB4, GSK3A, GSK3B, DISC1 |
| erbb4, gsk3a, nrxn1, bdnf | ErbB4, GSK3A, NRXN1, BDNF |
| erbb4, gsk3a, nrxn1, disc1 | ErbB4, GSK3A, NRXN1, DISC1 |
| erbb4, gsk3a, bdnf, disc1 | ErbB4, GSK3A, BDNF, DISC1 |
| erbb4, gsk3b, nrxn1, bdnf | ErbB4, GSK3B, NRXN1, BDNF |
| erbb4, gsk3b, nrxn1, disc1 | ErbB4, GSK3B, NRXN1, DISC1 |
| erbb4, gsk3b, bdnf, disc1 | ErbB4, GSK3B, BDNF, DISC1 |
| erbb4, nrxn1, bdnf, disc1 | ErbB4, NRXN1, BDNF, DISC1 |
| cplx1, tph1, tph2, gsk3a | CPLX1, TPH1, TPH2, GSK3A |
| cplx1, tph1, tph2, gsk3b | CPLX1, TPH1, TPH2, GSK3B |
| cplx1, tph1, tph2, nrxn1 | CPLX1, TPH1, TPH2, NRXN1 |
| cplx1, tph1, tph2, bdnf | CPLX1, TPH1, TPH2, BDNF |
| cplx1, tph1, tph2, disc1 | CPLX1, TPH1, TPH2, DISC1 |
| cplx1, tph1, gsk3a, gsk3b | CPLX1, TPH1, GSK3A, GSK3B |
| cplx1, tph1, gsk3a, nrxn1 | CPLX1, TPH1, GSK3A, NRXN1 |
| cplx1, tph1, gsk3a, bdnf | CPLX1, TPH1, GSK3A, BDNF |
| cplx1, tph1, gsk3a, disc1 | CPLX1, TPH1, GSK3A, DISC1 |
| cplx1, tph1, gsk3b, nrxn1 | CPLX1, TPH1, GSK3B, NRXN1 |
| cplx1, tph1, gsk3b, bdnf | CPLX1, TPH1, GSK3B, BDNF |
| cplx1, tph1, gsk3b, disc1 | CPLX1, TPH1, GSK3B, DISC1 |
| cplx1, tph1, nrxn1, bdnf | CPLX1, TPH1, NRXN1, BDNF |
| cplx1, tph1, nrxn1, disc1 | CPLX1, TPH1, NRXN1, DISC1 |
| cplx1, tph1, bdnf, disc1 | CPLX1, TPH1, BDNF, DISC1 |
| cplx1, tph2, gsk3a, gsk3b | CPLX1, TPH2, GSK3A, GSK3B |
| cplx1, tph2, gsk3a, nrxn1 | CPLX1, TPH2, GSK3A, NRXN1 |
| cplx1, tph2, gsk3a, bdnf | CPLX1, TPH2, GSK3A, BDNF |
| cplx1, tph2, gsk3a, disc1 | CPLX1, TPH2, GSK3A, DISC1 |
| cplx1, tph2, gsk3b, nrxn1 | CPLX1, TPH2, GSK3B, NRXN1 |
| cplx1, tph2, gsk3b, bdnf | CPLX1, TPH2, GSK3B, BDNF |
| cplx1, tph2, gsk3b, disc1 | CPLX1, TPH2, GSK3B, DISC1 |
| cplx1, tph2, nrxn1, bdnf | CPLX1, TPH2, NRXN1, BDNF |
| cplx1, tph2, nrxn1, disc1 | CPLX1, TPH2, NRXN1, DISC1 |
| cplx1, tph2, bdnf, disc1 | CPLX1, TPH2, BDNF, DISC1 |
| cplx1, gsk3a, gsk3b, nrxn1 | CPLX1, GSK3A, GSK3B, NRXN1 |
| cplx1, gsk3a, gsk3b, bdnf | CPLX1, GSK3A, GSK3B, BDNF |
| cplx1, gsk3a, gsk3b, disc1 | CPLX1, GSK3A, GSK3B, DISC1 |
| cplx1, gsk3a, nrxn1, bdnf | CPLX1, GSK3A, NRXN1, BDNF |
| cplx1, gsk3a, nrxn1, disc1 | CPLX1, GSK3A, NRXN1, DISC1 |
| cplx1, gsk3a, bdnf, disc1 | CPLX1, GSK3A, BDNF, DISC1 |
| cplx1, gsk3b, nrxn1, bdnf | CPLX1, GSK3B, NRXN1, BDNF |
| cplx1, gsk3b, nrxn1, disc1 | CPLX1, GSK3B, NRXN1, DISC1 |
| cplx1, gsk3b, bdnf, disc1 | CPLX1, GSK3B, BDNF, DISC1 |
| cplx1, nrxn1, bdnf, disc1 | CPLX1, NRXN1, BDNF, DISC1 |
| tph1, tph2, gsk3a, gsk3b | TPH1, TPH2, GSK3A, GSK3B |
| tph1, tph2, gsk3a, nrxn1 | TPH1, TPH2, GSK3A, NRXN1 |
| tph1, tph2, gsk3a, bdnf | TPH1, TPH2, GSK3A, BDNF |
| tph1, tph2, gsk3a, disc1 | TPH1, TPH2, GSK3A, DISC1 |
| tph1, tph2, gsk3b, nrxn1 | TPH1, TPH2, GSK3B, NRXN1 |
| tph1, tph2, gsk3b, bdnf | TPH1, TPH2, GSK3B, BDNF |
| tph1, tph2, gsk3b, disc1 | TPH1, TPH2, GSK3B, DISC1 |
| tph1, tph2, nrxn1, bdnf | TPH1, TPH2, NRXN1, BDNF |
| tph1, tph2, nrxn1, disc1 | TPH1, TPH2, NRXN1, DISC1 |
| tph1, tph2, bdnf, disc1 | TPH1, TPH2, BDNF, DISC1 |
| tph1, gsk3a, gsk3b, nrxn1 | TPH1, GSK3A, GSK3B, NRXN1 |
| tph1, gsk3a, gsk3b, bdnf | TPH1, GSK3A, GSK3B, BDNF |
| tph1, gsk3a, gsk3b, disc1 | TPH1, GSK3A, GSK3B, DISC1 |
| tph1, gsk3a, nrxn1, bdnf | TPH1, GSK3A, NRXN1, BDNF |
| tph1, gsk3a, nrxn1, disc1 | TPH1, GSK3A, NRXN1, DISC1 |
| tph1, gsk3a, bdnf, disc1 | TPH1, GSK3A, BDNF, DISC1 |
| tph1, gsk3b, nrxn1, bdnf | TPH1, GSK3B, NRXN1, BDNF |
| tph1, gsk3b, nrxn1, disc1 | TPH1, GSK3B, NRXN1, DISC1 |
| tph1, gsk3b, bdnf, disc1 | TPH1, GSK3B, BDNF, DISC1 |
| tph1, nrxn1, bdnf, disc1 | TPH1, NRXN1, BDNF, DISC1 |
| tph2, gsk3a, gsk3b, nrxn1 | TPH2, GSK3A, GSK3B, NRXN1 |
| tph2, gsk3a, gsk3b, bdnf | TPH2, GSK3A, GSK3B, BDNF |
| tph2, gsk3a, gsk3b, disc1 | TPH2, GSK3A, GSK3B, DISC1 |
| tph2, gsk3a, nrxn1, bdnf | TPH2, GSK3A, NRXN1, BDNF |
| tph2, gsk3a, nrxn1, disc1 | TPH2, GSK3A, NRXN1, DISC1 |
| tph2, gsk3a, bdnf, disc1 | TPH2, GSK3A, BDNF, DISC1 |
| tph2, gsk3b, nrxn1, bdnf | TPH2, GSK3B, NRXN1, BDNF |
| tph2, gsk3b, nrxn1, disc1 | TPH2, GSK3B, NRXN1, DISC1 |
| tph2, gsk3b, bdnf, disc1 | TPH2, GSK3B, BDNF, DISC1 |
| tph2, nrxn1, bdnf, disc1 | TPH2, NRXN1, BDNF, DISC1 |
| gsk3a, gsk3b, nrxn1, bdnf | GSK3A, GSK3B, NRXN1, BDNF |
| gsk3a, gsk3b, nrxn1, disc1 | GSK3A, GSK3B, NRXN1, DISC1 |
| gsk3a, gsk3b, bdnf, disc1 | GSK3A, GSK3B, BDNF, DISC1 |
| gsk3a, nrxn1, bdnf, disc1 | GSK3A, NRXN1, BDNF, DISC1 |
| gsk3b, nrxn1, bdnf, disc1 | GSK3B, NRXN1, BDNF, DISC1 |
| nrg1, erbb4, cplx1, tph1, tph2 | NRG1, ErbB4, CPLX1, TPH1, TPH2 |
| nrg1, erbb4, cplx1, tph1, gsk3a | NRG1, ErbB4, CPLX1, TPH1, GSK3A |
| nrg1, erbb4, cplx1, tph1, gsk3b | NRG1, ErbB4, CPLX1, TPH1, GSK3B |
| nrg1, erbb4, cplx1, tph1, nrxn1 | NRG1, ErbB4, CPLX1, TPH1, NRXN1 |
| nrg1, erbb4, cplx1, tph1, bdnf | NRG1, ErbB4, CPLX1, TPH1, BDNF |
| nrg1, erbb4, cplx1, tph1, disc1 | NRG1, ErbB4, CPLX1, TPH1, DISC1 |
| nrg1, erbb4, cplx1, tph2, gsk3a | NRG1, ErbB4, CPLX1, TPH2, GSK3A |
| nrg1, erbb4, cplx1, tph2, gsk3b | NRG1, ErbB4, CPLX1, TPH2, GSK3B |
| nrg1, erbb4, cplx1, tph2, nrxn1 | NRG1, ErbB4, CPLX1, TPH2, NRXN1 |
| nrg1, erbb4, cplx1, tph2, bdnf | NRG1, ErbB4, CPLX1, TPH2, BDNF |
| nrg1, erbb4, cplx1, tph2, disc1 | NRG1, ErbB4, CPLX1, TPH2, DISC1 |
| nrg1, erbb4, cplx1, gsk3a, gsk3b | NRG1, ErbB4, CPLX1, GSK3A, GSK3B |
| nrg1, erbb4, cplx1, gsk3a, nrxn1 | NRG1, ErbB4, CPLX1, GSK3A, NRXN1 |
| nrg1, erbb4, cplx1, gsk3a, bdnf | NRG1, ErbB4, CPLX1, GSK3A, BDNF |
| nrg1, erbb4, cplx1, gsk3a, disc1 | NRG1, ErbB4, CPLX1, GSK3A, DISC1 |
| nrg1, erbb4, cplx1, gsk3b, nrxn1 | NRG1, ErbB4, CPLX1, GSK3B, NRXN1 |
| nrg1, erbb4, cplx1, gsk3b, bdnf | NRG1, ErbB4, CPLX1, GSK3B, BDNF |
| nrg1, erbb4, cplx1, gsk3b, disc1 | NRG1, ErbB4, CPLX1, GSK3B, DISC1 |
| nrg1, erbb4, cplx1, nrxn1, bdnf | NRG1, ErbB4, CPLX1, NRXN1, BDNF |
| nrg1, erbb4, cplx1, nrxn1, disc1 | NRG1, ErbB4, CPLX1, NRXN1, DISC1 |
| nrg1, erbb4, cplx1, bdnf, disc1 | NRG1, ErbB4, CPLX1, BDNF, DISC1 |
| nrg1, erbb4, tph1, tph2, gsk3a | NRG1, ErbB4, TPH1, TPH2, GSK3A |
| nrg1, erbb4, tph1, tph2, gsk3b | NRG1, ErbB4, TPH1, TPH2, GSK3B |
| nrg1, erbb4, tph1, tph2, nrxn1 | NRG1, ErbB4, TPH1, TPH2, NRXN1 |
| nrg1, erbb4, tph1, tph2, bdnf | NRG1, ErbB4, TPH1, TPH2, BDNF |
| nrg1, erbb4, tph1, tph2, disc1 | NRG1, ErbB4, TPH1, TPH2, DISC1 |
| nrg1, erbb4, tph1, gsk3a, gsk3b | NRG1, ErbB4, TPH1, GSK3A, GSK3B |
| nrg1, erbb4, tph1, gsk3a, nrxn1 | NRG1, ErbB4, TPH1, GSK3A, NRXN1 |
| nrg1, erbb4, tph1, gsk3a, bdnf | NRG1, ErbB4, TPH1, GSK3A, BDNF |
| nrg1, erbb4, tph1, gsk3a, disc1 | NRG1, ErbB4, TPH1, GSK3A, DISC1 |
| nrg1, erbb4, tph1, gsk3b, nrxn1 | NRG1, ErbB4, TPH1, GSK3B, NRXN1 |
| nrg1, erbb4, tph1, gsk3b, bdnf | NRG1, ErbB4, TPH1, GSK3B, BDNF |
| nrg1, erbb4, tph1, gsk3b, disc1 | NRG1, ErbB4, TPH1, GSK3B, DISC1 |
| nrg1, erbb4, tph1, nrxn1, bdnf | NRG1, ErbB4, TPH1, NRXN1, BDNF |
| nrg1, erbb4, tph1, nrxn1, disc1 | NRG1, ErbB4, TPH1, NRXN1, DISC1 |
| nrg1, erbb4, tph1, bdnf, disc1 | NRG1, ErbB4, TPH1, BDNF, DISC1 |
| nrg1, erbb4, tph2, gsk3a, gsk3b | NRG1, ErbB4, TPH2, GSK3A, GSK3B |
| nrg1, erbb4, tph2, gsk3a, nrxn1 | NRG1, ErbB4, TPH2, GSK3A, NRXN1 |
| nrg1, erbb4, tph2, gsk3a, bdnf | NRG1, ErbB4, TPH2, GSK3A, BDNF |
| nrg1, erbb4, tph2, gsk3a, disc1 | NRG1, ErbB4, TPH2, GSK3A, DISC1 |
| nrg1, erbb4, tph2, gsk3b, nrxn1 | NRG1, ErbB4, TPH2, GSK3B, NRXN1 |
| nrg1, erbb4, tph2, gsk3b, bdnf | NRG1, ErbB4, TPH2, GSK3B, BDNF |
| nrg1, erbb4, tph2, gsk3b, disc1 | NRG1, ErbB4, TPH2, GSK3B, DISC1 |
| nrg1, erbb4, tph2, nrxn1, bdnf | NRG1, ErbB4, TPH2, NRXN1, BDNF |
| nrg1, erbb4, tph2, nrxn1, disc1 | NRG1, ErbB4, TPH2, NRXN1, DISC1 |
| nrg1, erbb4, tph2, bdnf, disc1 | NRG1, ErbB4, TPH2, BDNF, DISC1 |
| nrg1, erbb4, gsk3a, gsk3b, nrxn1 | NRG1, ErbB4, GSK3A, GSK3B, NRXN1 |
| nrg1, erbb4, gsk3a, gsk3b, bdnf | NRG1, ErbB4, GSK3A, GSK3B, BDNF |
| nrg1, erbb4, gsk3a, gsk3b, disc1 | NRG1, ErbB4, GSK3A, GSK3B, DISC1 |
| nrg1, erbb4, gsk3a, nrxn1, bdnf | NRG1, ErbB4, GSK3A, NRXN1, BDNF |
| nrg1, erbb4, gsk3a, nrxn1, disc1 | NRG1, ErbB4, GSK3A, NRXN1, DISC1 |
| nrg1, erbb4, gsk3a, bdnf, disc1 | NRG1, ErbB4, GSK3A, BDNF, DISC1 |
| nrg1, erbb4, gsk3b, nrxn1, bdnf | NRG1, ErbB4, GSK3B, NRXN1, BDNF |
| nrg1, erbb4, gsk3b, nrxn1, disc1 | NRG1, ErbB4, GSK3B, NRXN1, DISC1 |
| nrg1, erbb4, gsk3b, bdnf, disc1 | NRG1, ErbB4, GSK3B, BDNF, DISC1 |
| nrg1, erbb4, nrxn1, bdnf, disc1 | NRG1, ErbB4, NRXN1, BDNF, DISC1 |
| nrg1, cplx1, tph1, tph2, gsk3a | NRG1, CPLX1, TPH1, TPH2, GSK3A |
| nrg1, cplx1, tph1, tph2, gsk3b | NRG1, CPLX1, TPH1, TPH2, GSK3B |
| nrg1, cplx1, tph1, tph2, nrxn1 | NRG1, CPLX1, TPH1, TPH2, NRXN1 |
| nrg1, cplx1, tph1, tph2, bdnf | NRG1, CPLX1, TPH1, TPH2, BDNF |
| nrg1, cplx1, tph1, tph2, disc1 | NRG1, CPLX1, TPH1, TPH2, DISC1 |
| nrg1, cplx1, tph1, gsk3a, gsk3b | NRG1, CPLX1, TPH1, GSK3A, GSK3B |
| nrg1, cplx1, tph1, gsk3a, nrxn1 | NRG1, CPLX1, TPH1, GSK3A, NRXN1 |
| nrg1, cplx1, tph1, gsk3a, bdnf | NRG1, CPLX1, TPH1, GSK3A, BDNF |
| nrg1, cplx1, tph1, gsk3a, disc1 | NRG1, CPLX1, TPH1, GSK3A, DISC1 |
| nrg1, cplx1, tph1, gsk3b, nrxn1 | NRG1, CPLX1, TPH1, GSK3B, NRXN1 |
| nrg1, cplx1, tph1, gsk3b, bdnf | NRG1, CPLX1, TPH1, GSK3B, BDNF |
| nrg1, cplx1, tph1, gsk3b, disc1 | NRG1, CPLX1, TPH1, GSK3B, DISC1 |
| nrg1, cplx1, tph1, nrxn1, bdnf | NRG1, CPLX1, TPH1, NRXN1, BDNF |
| nrg1, cplx1, tph1, nrxn1, disc1 | NRG1, CPLX1, TPH1, NRXN1, DISC1 |
| nrg1, cplx1, tph1, bdnf, disc1 | NRG1, CPLX1, TPH1, BDNF, DISC1 |
| nrg1, cplx1, tph2, gsk3a, gsk3b | NRG1, CPLX1, TPH2, GSK3A, GSK3B |
| nrg1, cplx1, tph2, gsk3a, nrxn1 | NRG1, CPLX1, TPH2, GSK3A, NRXN1 |
| nrg1, cplx1, tph2, gsk3a, bdnf | NRG1, CPLX1, TPH2, GSK3A, BDNF |
| nrg1, cplx1, tph2, gsk3a, disc1 | NRG1, CPLX1, TPH2, GSK3A, DISC1 |
| nrg1, cplx1, tph2, gsk3b, nrxn1 | NRG1, CPLX1, TPH2, GSK3B, NRXN1 |
| nrg1, cplx1, tph2, gsk3b, bdnf | NRG1, CPLX1, TPH2, GSK3B, BDNF |
| nrg1, cplx1, tph2, gsk3b, disc1 | NRG1, CPLX1, TPH2, GSK3B, DISC1 |
| nrg1, cplx1, tph2, nrxn1, bdnf | NRG1, CPLX1, TPH2, NRXN1, BDNF |
| nrg1, cplx1, tph2, nrxn1, disc1 | NRG1, CPLX1, TPH2, NRXN1, DISC1 |
| nrg1, cplx1, tph2, bdnf, disc1 | NRG1, CPLX1, TPH2, BDNF, DISC1 |
| nrg1, cplx1, gsk3a, gsk3b, nrxn1 | NRG1, CPLX1, GSK3A, GSK3B, NRXN1 |
| nrg1, cplx1, gsk3a, gsk3b, bdnf | NRG1, CPLX1, GSK3A, GSK3B, BDNF |
| nrg1, cplx1, gsk3a, gsk3b, disc1 | NRG1, CPLX1, GSK3A, GSK3B, DISC1 |
| nrg1, cplx1, gsk3a, nrxn1, bdnf | NRG1, CPLX1, GSK3A, NRXN1, BDNF |
| nrg1, cplx1, gsk3a, nrxn1, disc1 | NRG1, CPLX1, GSK3A, NRXN1, DISC1 |
| nrg1, cplx1, gsk3a, bdnf, disc1 | NRG1, CPLX1, GSK3A, BDNF, DISC1 |
| nrg1, cplx1, gsk3b, nrxn1, bdnf | NRG1, CPLX1, GSK3B, NRXN1, BDNF |
| nrg1, cplx1, gsk3b, nrxn1, disc1 | NRG1, CPLX1, GSK3B, NRXN1, DISC1 |
| nrg1, cplx1, gsk3b, bdnf, disc1 | NRG1, CPLX1, GSK3B, BDNF, DISC1 |
| nrg1, cplx1, nrxn1, bdnf, disc1 | NRG1, CPLX1, NRXN1, BDNF, DISC1 |
| nrg1, tph1, tph2, gsk3a, gsk3b | NRG1, TPH1, TPH2, GSK3A, GSK3B |
| nrg1, tph1, tph2, gsk3a, nrxn1 | NRG1, TPH1, TPH2, GSK3A, NRXN1 |
| nrg1, tph1, tph2, gsk3a, bdnf | NRG1, TPH1, TPH2, GSK3A, BDNF |
| nrg1, tph1, tph2, gsk3a, disc1 | NRG1, TPH1, TPH2, GSK3A, DISC1 |
| nrg1, tph1, tph2, gsk3b, nrxn1 | NRG1, TPH1, TPH2, GSK3B, NRXN1 |
| nrg1, tph1, tph2, gsk3b, bdnf | NRG1, TPH1, TPH2, GSK3B, BDNF |
| nrg1, tph1, tph2, gsk3b, disc1 | NRG1, TPH1, TPH2, GSK3B, DISC1 |
| nrg1, tph1, tph2, nrxn1, bdnf | NRG1, TPH1, TPH2, NRXN1, BDNF |
| nrg1, tph1, tph2, nrxn1, disc1 | NRG1, TPH1, TPH2, NRXN1, DISC1 |
| nrg1, tph1, tph2, bdnf, disc1 | NRG1, TPH1, TPH2, BDNF, DISC1 |
| nrg1, tph1, gsk3a, gsk3b, nrxn1 | NRG1, TPH1, GSK3A, GSK3B, NRXN1 |
| nrg1, tph1, gsk3a, gsk3b, bdnf | NRG1, TPH1, GSK3A, GSK3B, BDNF |
| nrg1, tph1, gsk3a, gsk3b, disc1 | NRG1, TPH1, GSK3A, GSK3B, DISC1 |
| nrg1, tph1, gsk3a, nrxn1, bdnf | NRG1, TPH1, GSK3A, NRXN1, BDNF |
| nrg1, tph1, gsk3a, nrxn1, disc1 | NRG1, TPH1, GSK3A, NRXN1, DISC1 |
| nrg1, tph1, gsk3a, bdnf, disc1 | NRG1, TPH1, GSK3A, BDNF, DISC1 |
| nrg1, tph1, gsk3b, nrxn1, bdnf | NRG1, TPH1, GSK3B, NRXN1, BDNF |
| nrg1, tph1, gsk3b, nrxn1, disc1 | NRG1, TPH1, GSK3B, NRXN1, DISC1 |
| nrg1, tph1, gsk3b, bdnf, disc1 | NRG1, TPH1, GSK3B, BDNF, DISC1 |
| nrg1, tph1, nrxn1, bdnf, disc1 | NRG1, TPH1, NRXN1, BDNF, DISC1 |
| nrg1, tph2, gsk3a, gsk3b, nrxn1 | NRG1, TPH2, GSK3A, GSK3B, NRXN1 |
| nrg1, tph2, gsk3a, gsk3b, bdnf | NRG1, TPH2, GSK3A, GSK3B, BDNF |
| nrg1, tph2, gsk3a, gsk3b, disc1 | NRG1, TPH2, GSK3A, GSK3B, DISC1 |
| nrg1, tph2, gsk3a, nrxn1, bdnf | NRG1, TPH2, GSK3A, NRXN1, BDNF |
| nrg1, tph2, gsk3a, nrxn1, disc1 | NRG1, TPH2, GSK3A, NRXN1, DISC1 |
| nrg1, tph2, gsk3a, bdnf, disc1 | NRG1, TPH2, GSK3A, BDNF, DISC1 |
| nrg1, tph2, gsk3b, nrxn1, bdnf | NRG1, TPH2, GSK3B, NRXN1, BDNF |
| nrg1, tph2, gsk3b, nrxn1, disc1 | NRG1, TPH2, GSK3B, NRXN1, DISC1 |
| nrg1, tph2, gsk3b, bdnf, disc1 | NRG1, TPH2, GSK3B, BDNF, DISC1 |
| nrg1, tph2, nrxn1, bdnf, disc1 | NRG1, TPH2, NRXN1, BDNF, DISC1 |
| nrg1, gsk3a, gsk3b, nrxn1, bdnf | NRG1, GSK3A, GSK3B, NRXN1, BDNF |
| nrg1, gsk3a, gsk3b, nrxn1, disc1 | NRG1, GSK3A, GSK3B, NRXN1, DISC1 |
| nrg1, gsk3a, gsk3b, bdnf, disc1 | NRG1, GSK3A, GSK3B, BDNF, DISC1 |
| nrg1, gsk3a, nrxn1, bdnf, disc1 | NRG1, GSK3A, NRXN1, BDNF, DISC1 |
| nrg1, gsk3b, nrxn1, bdnf, disc1 | NRG1, GSK3B, NRXN1, BDNF, DISC1 |
| erbb4, cplx1, tph1, tph2, gsk3a | ErbB4, CPLX1, TPH1, TPH2, GSK3A |
| erbb4, cplx1, tph1, tph2, gsk3b | ErbB4, CPLX1, TPH1, TPH2, GSK3B |
| erbb4, cplx1, tph1, tph2, nrxn1 | ErbB4, CPLX1, TPH1, TPH2, NRXN1 |
| erbb4, cplx1, tph1, tph2, bdnf | ErbB4, CPLX1, TPH1, TPH2, BDNF |
| erbb4, cplx1, tph1, tph2, disc1 | ErbB4, CPLX1, TPH1, TPH2, DISC1 |
| erbb4, cplx1, tph1, gsk3a, gsk3b | ErbB4, CPLX1, TPH1, GSK3A, GSK3B |
| erbb4, cplx1, tph1, gsk3a, nrxn1 | ErbB4, CPLX1, TPH1, GSK3A, NRXN1 |
| erbb4, cplx1, tph1, gsk3a, bdnf | ErbB4, CPLX1, TPH1, GSK3A, BDNF |
| erbb4, cplx1, tph1, gsk3a, disc1 | ErbB4, CPLX1, TPH1, GSK3A, DISC1 |
| erbb4, cplx1, tph1, gsk3b, nrxn1 | ErbB4, CPLX1, TPH1, GSK3B, NRXN1 |
| erbb4, cplx1, tph1, gsk3b, bdnf | ErbB4, CPLX1, TPH1, GSK3B, BDNF |
| erbb4, cplx1, tph1, gsk3b, disc1 | ErbB4, CPLX1, TPH1, GSK3B, DISC1 |
| erbb4, cplx1, tph1, nrxn1, bdnf | ErbB4, CPLX1, TPH1, NRXN1, BDNF |
| erbb4, cplx1, tph1, nrxn1, disc1 | ErbB4, CPLX1, TPH1, NRXN1, DISC1 |
| erbb4, cplx1, tph1, bdnf, disc1 | ErbB4, CPLX1, TPH1, BDNF, DISC1 |
| erbb4, cplx1, tph2, gsk3a, gsk3b | ErbB4, CPLX1, TPH2, GSK3A, GSK3B |
| erbb4, cplx1, tph2, gsk3a, nrxn1 | ErbB4, CPLX1, TPH2, GSK3A, NRXN1 |
| erbb4, cplx1, tph2, gsk3a, bdnf | ErbB4, CPLX1, TPH2, GSK3A, BDNF |
| erbb4, cplx1, tph2, gsk3a, disc1 | ErbB4, CPLX1, TPH2, GSK3A, DISC1 |
| erbb4, cplx1, tph2, gsk3b, nrxn1 | ErbB4, CPLX1, TPH2, GSK3B, NRXN1 |
| erbb4, cplx1, tph2, gsk3b, bdnf | ErbB4, CPLX1, TPH2, GSK3B, BDNF |
| erbb4, cplx1, tph2, gsk3b, disc1 | ErbB4, CPLX1, TPH2, GSK3B, DISC1 |
| erbb4, cplx1, tph2, nrxn1, bdnf | ErbB4, CPLX1, TPH2, NRXN1, BDNF |
| erbb4, cplx1, tph2, nrxn1, disc1 | ErbB4, CPLX1, TPH2, NRXN1, DISC1 |
| erbb4, cplx1, tph2, bdnf, disc1 | ErbB4, CPLX1, TPH2, BDNF, DISC1 |
| erbb4, cplx1, gsk3a, gsk3b, nrxn1 | ErbB4, CPLX1, GSK3A, GSK3B, NRXN1 |
| erbb4, cplx1, gsk3a, gsk3b, bdnf | ErbB4, CPLX1, GSK3A, GSK3B, BDNF |
| erbb4, cplx1, gsk3a, gsk3b, disc1 | ErbB4, CPLX1, GSK3A, GSK3B, DISC1 |
| erbb4, cplx1, gsk3a, nrxn1, bdnf | ErbB4, CPLX1, GSK3A, NRXN1, BDNF |
| erbb4, cplx1, gsk3a, nrxn1, disc1 | ErbB4, CPLX1, GSK3A, NRXN1, DISC1 |
| erbb4, cplx1, gsk3a, bdnf, disc1 | ErbB4, CPLX1, GSK3A, BDNF, DISC1 |
| erbb4, cplx1, gsk3b, nrxn1, bdnf | ErbB4, CPLX1, GSK3B, NRXN1, BDNF |
| erbb4, cplx1, gsk3b, nrxn1, disc1 | ErbB4, CPLX1, GSK3B, NRXN1, DISC1 |
| erbb4, cplx1, gsk3b, bdnf, disc1 | ErbB4, CPLX1, GSK3B, BDNF, DISC1 |
| erbb4, cplx1, nrxn1, bdnf, disc1 | ErbB4, CPLX1, NRXN1, BDNF, DISC1 |
| erbb4, tph1, tph2, gsk3a, gsk3b | ErbB4, TPH1, TPH2, GSK3A, GSK3B |
| erbb4, tph1, tph2, gsk3a, nrxn1 | ErbB4, TPH1, TPH2, GSK3A, NRXN1 |
| erbb4, tph1, tph2, gsk3a, bdnf | ErbB4, TPH1, TPH2, GSK3A, BDNF |
| erbb4, tph1, tph2, gsk3a, disc1 | ErbB4, TPH1, TPH2, GSK3A, DISC1 |
| erbb4, tph1, tph2, gsk3b, nrxn1 | ErbB4, TPH1, TPH2, GSK3B, NRXN1 |
| erbb4, tph1, tph2, gsk3b, bdnf | ErbB4, TPH1, TPH2, GSK3B, BDNF |
| erbb4, tph1, tph2, gsk3b, disc1 | ErbB4, TPH1, TPH2, GSK3B, DISC1 |
| erbb4, tph1, tph2, nrxn1, bdnf | ErbB4, TPH1, TPH2, NRXN1, BDNF |
| erbb4, tph1, tph2, nrxn1, disc1 | ErbB4, TPH1, TPH2, NRXN1, DISC1 |
| erbb4, tph1, tph2, bdnf, disc1 | ErbB4, TPH1, TPH2, BDNF, DISC1 |
| erbb4, tph1, gsk3a, gsk3b, nrxn1 | ErbB4, TPH1, GSK3A, GSK3B, NRXN1 |
| erbb4, tph1, gsk3a, gsk3b, bdnf | ErbB4, TPH1, GSK3A, GSK3B, BDNF |
| erbb4, tph1, gsk3a, gsk3b, disc1 | ErbB4, TPH1, GSK3A, GSK3B, DISC1 |
| erbb4, tph1, gsk3a, nrxn1, bdnf | ErbB4, TPH1, GSK3A, NRXN1, BDNF |
| erbb4, tph1, gsk3a, nrxn1, disc1 | ErbB4, TPH1, GSK3A, NRXN1, DISC1 |
| erbb4, tph1, gsk3a, bdnf, disc1 | ErbB4, TPH1, GSK3A, BDNF, DISC1 |
| erbb4, tph1, gsk3b, nrxn1, bdnf | ErbB4, TPH1, GSK3B, NRXN1, BDNF |
| erbb4, tph1, gsk3b, nrxn1, disc1 | ErbB4, TPH1, GSK3B, NRXN1, DISC1 |
| erbb4, tph1, gsk3b, bdnf, disc1 | ErbB4, TPH1, GSK3B, BDNF, DISC1 |
| erbb4, tph1, nrxn1, bdnf, disc1 | ErbB4, TPH1, NRXN1, BDNF, DISC1 |
| erbb4, tph2, gsk3a, gsk3b, nrxn1 | ErbB4, TPH2, GSK3A, GSK3B, NRXN1 |
| erbb4, tph2, gsk3a, gsk3b, bdnf | ErbB4, TPH2, GSK3A, GSK3B, BDNF |
| erbb4, tph2, gsk3a, gsk3b, disc1 | ErbB4, TPH2, GSK3A, GSK3B, DISC1 |
| erbb4, tph2, gsk3a, nrxn1, bdnf | ErbB4, TPH2, GSK3A, NRXN1, BDNF |
| erbb4, tph2, gsk3a, nrxn1, disc1 | ErbB4, TPH2, GSK3A, NRXN1, DISC1 |
| erbb4, tph2, gsk3a, bdnf, disc1 | ErbB4, TPH2, GSK3A, BDNF, DISC1 |
| erbb4, tph2, gsk3b, nrxn1, bdnf | ErbB4, TPH2, GSK3B, NRXN1, BDNF |
| erbb4, tph2, gsk3b, nrxn1, disc1 | ErbB4, TPH2, GSK3B, NRXN1, DISC1 |
| erbb4, tph2, gsk3b, bdnf, disc1 | ErbB4, TPH2, GSK3B, BDNF, DISC1 |
| erbb4, tph2, nrxn1, bdnf, disc1 | ErbB4, TPH2, NRXN1, BDNF, DISC1 |
| erbb4, gsk3a, gsk3b, nrxn1, bdnf | ErbB4, GSK3A, GSK3B, NRXN1, BDNF |
| erbb4, gsk3a, gsk3b, nrxn1, disc1 | ErbB4, GSK3A, GSK3B, NRXN1, DISC1 |
| erbb4, gsk3a, gsk3b, bdnf, disc1 | ErbB4, GSK3A, GSK3B, BDNF, DISC1 |
| erbb4, gsk3a, nrxn1, bdnf, disc1 | ErbB4, GSK3A, NRXN1, BDNF, DISC1 |
| erbb4, gsk3b, nrxn1, bdnf, disc1 | ErbB4, GSK3B, NRXN1, BDNF, DISC1 |
| cplx1, tph1, tph2, gsk3a, gsk3b | CPLX1, TPH1, TPH2, GSK3A, GSK3B |
| cplx1, tph1, tph2, gsk3a, nrxn1 | CPLX1, TPH1, TPH2, GSK3A, NRXN1 |
| cplx1, tph1, tph2, gsk3a, bdnf | CPLX1, TPH1, TPH2, GSK3A, BDNF |
| cplx1, tph1, tph2, gsk3a, disc1 | CPLX1, TPH1, TPH2, GSK3A, DISC1 |
| cplx1, tph1, tph2, gsk3b, nrxn1 | CPLX1, TPH1, TPH2, GSK3B, NRXN1 |
| cplx1, tph1, tph2, gsk3b, bdnf | CPLX1, TPH1, TPH2, GSK3B, BDNF |
| cplx1, tph1, tph2, gsk3b, disc1 | CPLX1, TPH1, TPH2, GSK3B, DISC1 |
| cplx1, tph1, tph2, nrxn1, bdnf | CPLX1, TPH1, TPH2, NRXN1, BDNF |
| cplx1, tph1, tph2, nrxn1, disc1 | CPLX1, TPH1, TPH2, NRXN1, DISC1 |
| cplx1, tph1, tph2, bdnf, disc1 | CPLX1, TPH1, TPH2, BDNF, DISC1 |
| cplx1, tph1, gsk3a, gsk3b, nrxn1 | CPLX1, TPH1, GSK3A, GSK3B, NRXN1 |
| cplx1, tph1, gsk3a, gsk3b, bdnf | CPLX1, TPH1, GSK3A, GSK3B, BDNF |
| cplx1, tph1, gsk3a, gsk3b, disc1 | CPLX1, TPH1, GSK3A, GSK3B, DISC1 |
| cplx1, tph1, gsk3a, nrxn1, bdnf | CPLX1, TPH1, GSK3A, NRXN1, BDNF |
| cplx1, tph1, gsk3a, nrxn1, disc1 | CPLX1, TPH1, GSK3A, NRXN1, DISC1 |
| cplx1, tph1, gsk3a, bdnf, disc1 | CPLX1, TPH1, GSK3A, BDNF, DISC1 |
| cplx1, tph1, gsk3b, nrxn1, bdnf | CPLX1, TPH1, GSK3B, NRXN1, BDNF |
| cplx1, tph1, gsk3b, nrxn1, disc1 | CPLX1, TPH1, GSK3B, NRXN1, DISC1 |
| cplx1, tph1, gsk3b, bdnf, disc1 | CPLX1, TPH1, GSK3B, BDNF, DISC1 |
| cplx1, tph1, nrxn1, bdnf, disc1 | CPLX1, TPH1, NRXN1, BDNF, DISC1 |
| cplx1, tph2, gsk3a, gsk3b, nrxn1 | CPLX1, TPH2, GSK3A, GSK3B, NRXN1 |
| cplx1, tph2, gsk3a, gsk3b, bdnf | CPLX1, TPH2, GSK3A, GSK3B, BDNF |
| cplx1, tph2, gsk3a, gsk3b, disc1 | CPLX1, TPH2, GSK3A, GSK3B, DISC1 |
| cplx1, tph2, gsk3a, nrxn1, bdnf | CPLX1, TPH2, GSK3A, NRXN1, BDNF |
| cplx1, tph2, gsk3a, nrxn1, disc1 | CPLX1, TPH2, GSK3A, NRXN1, DISC1 |
| cplx1, tph2, gsk3a, bdnf, disc1 | CPLX1, TPH2, GSK3A, BDNF, DISC1 |
| cplx1, tph2, gsk3b, nrxn1, bdnf | CPLX1, TPH2, GSK3B, NRXN1, BDNF |
| cplx1, tph2, gsk3b, nrxn1, disc1 | CPLX1, TPH2, GSK3B, NRXN1, DISC1 |
| cplx1, tph2, gsk3b, bdnf, disc1 | CPLX1, TPH2, GSK3B, BDNF, DISC1 |
| cplx1, tph2, nrxn1, bdnf, disc1 | CPLX1, TPH2, NRXN1, BDNF, DISC1 |
| cplx1, gsk3a, gsk3b, nrxn1, bdnf | CPLX1, GSK3A, GSK3B, NRXN1, BDNF |
| cplx1, gsk3a, gsk3b, nrxn1, disc1 | CPLX1, GSK3A, GSK3B, NRXN1, DISC1 |
| cplx1, gsk3a, gsk3b, bdnf, disc1 | CPLX1, GSK3A, GSK3B, BDNF, DISC1 |
| cplx1, gsk3a, nrxn1, bdnf, disc1 | CPLX1, GSK3A, NRXN1, BDNF, DISC1 |
| cplx1, gsk3b, nrxn1, bdnf, disc1 | CPLX1, GSK3B, NRXN1, BDNF, DISC1 |
| tph1, tph2, gsk3a, gsk3b, nrxn1 | TPH1, TPH2, GSK3A, GSK3B, NRXN1 |
| tph1, tph2, gsk3a, gsk3b, bdnf | TPH1, TPH2, GSK3A, GSK3B, BDNF |
| tph1, tph2, gsk3a, gsk3b, disc1 | TPH1, TPH2, GSK3A, GSK3B, DISC1 |
| tph1, tph2, gsk3a, nrxn1, bdnf | TPH1, TPH2, GSK3A, NRXN1, BDNF |
| tph1, tph2, gsk3a, nrxn1, disc1 | TPH1, TPH2, GSK3A, NRXN1, DISC1 |
| tph1, tph2, gsk3a, bdnf, disc1 | TPH1, TPH2, GSK3A, BDNF, DISC1 |
| tph1, tph2, gsk3b, nrxn1, bdnf | TPH1, TPH2, GSK3B, NRXN1, BDNF |
| tph1, tph2, gsk3b, nrxn1, disc1 | TPH1, TPH2, GSK3B, NRXN1, DISC1 |
| tph1, tph2, gsk3b, bdnf, disc1 | TPH1, TPH2, GSK3B, BDNF, DISC1 |
| tph1, tph2, nrxn1, bdnf, disc1 | TPH1, TPH2, NRXN1, BDNF, DISC1 |
| tph1, gsk3a, gsk3b, nrxn1, bdnf | TPH1, GSK3A, GSK3B, NRXN1, BDNF |
| tph1, gsk3a, gsk3b, nrxn1, disc1 | TPH1, GSK3A, GSK3B, NRXN1, DISC1 |
| tph1, gsk3a, gsk3b, bdnf, disc1 | TPH1, GSK3A, GSK3B, BDNF, DISC1 |
| tph1, gsk3a, nrxn1, bdnf, disc1 | TPH1, GSK3A, NRXN1, BDNF, DISC1 |
| tph1, gsk3b, nrxn1, bdnf, disc1 | TPH1, GSK3B, NRXN1, BDNF, DISC1 |
| tph2, gsk3a, gsk3b, nrxn1, bdnf | TPH2, GSK3A, GSK3B, NRXN1, BDNF |
| tph2, gsk3a, gsk3b, nrxn1, disc1 | TPH2, GSK3A, GSK3B, NRXN1, DISC1 |
| tph2, gsk3a, gsk3b, bdnf, disc1 | TPH2, GSK3A, GSK3B, BDNF, DISC1 |
| tph2, gsk3a, nrxn1, bdnf, disc1 | TPH2, GSK3A, NRXN1, BDNF, DISC1 |
| tph2, gsk3b, nrxn1, bdnf, disc1 | TPH2, GSK3B, NRXN1, BDNF, DISC1 |
| gsk3a, gsk3b, nrxn1, bdnf, disc1 | GSK3A, GSK3B, NRXN1, BDNF, DISC1 |
| nrg1, erbb4, cplx1, tph1, tph2, gsk3a | NRG1, ErbB4, CPLX1, TPH1, TPH2, |
| | GSK3A |
| nrg1, erbb4, cplx1, tph1, tph2, gsk3b | NRG1, ErbB4, CPLX1, TPH1, TPH2, |
| | GSK3B |
| nrg1, erbb4, cplx1, tph1, tph2, nrxn1 | NRG1, ErbB4, CPLX1, TPH1, TPH2, |
| | NRXN1 |
| nrg1, erbb4, cplx1, tph1, tph2, bdnf | NRG1, ErbB4, CPLX1, TPH1, TPH2, |
| | BDNF |
| nrg1, erbb4, cplx1, tph1, tph2, disc1 | NRG1, ErbB4, CPLX1, TPH1, TPH2, |
| | DISC1 |
| nrg1, erbb4, cplx1, tph1, gsk3a, gsk3b | NRG1, ErbB4, CPLX1, TPH1, GSK3A, |
| | GSK3B |
| nrg1, erbb4, cplx1, tph1, gsk3a, nrxn1 | NRG1, ErbB4, CPLX1, TPH1, GSK3A, |
| | NRXN1 |
| nrg1, erbb4, cplx1, tph1, gsk3a, bdnf | NRG1, ErbB4, CPLX1, TPH1, GSK3A, |
| | BDNF |
| nrg1, erbb4, cplx1, tph1, gsk3a, disc1 | NRG1, ErbB4, CPLX1, TPH1, GSK3A, |
| | DISC1 |
| nrg1, erbb4, cplx1, tph1, gsk3b, nrxn1 | NRG1, ErbB4, CPLX1, TPH1, GSK3B, |
| | NRXN1 |
| nrg1, erbb4, cplx1, tph1, gsk3b, bdnf | NRG1, ErbB4, CPLX1, TPH1, GSK3B, |
| | BDNF |
| nrg1, erbb4, cplx1, tph1, gsk3b, disc1 | NRG1, ErbB4, CPLX1, TPH1, GSK3B, |
| | DISC1 |
| nrg1, erbb4, cplx1, tph1, nrxn1, bdnf | NRG1, ErbB4, CPLX1, TPH1, NRXN1, |
| | BDNF |
| nrg1, erbb4, cplx1, tph1, nrxn1, disc1 | NRG1, ErbB4, CPLX1, TPH1, NRXN1, |
| | DISC1 |
| nrg1, erbb4, cplx1, tph1, bdnf, disc1 | NRG1, ErbB4, CPLX1, TPH1, BDNF, |
| | DISC1 |
| nrg1, erbb4, cplx1, tph2, gsk3a, gsk3b | NRG1, ErbB4, CPLX1, TPH2, GSK3A, |
| | GSK3B |
| nrg1, erbb4, cplx1, tph2, gsk3a, nrxn1 | NRG1, ErbB4, CPLX1, TPH2, GSK3A, |
| | NRXN1 |
| nrg1, erbb4, cplx1, tph2, gsk3a, bdnf | NRG1, ErbB4, CPLX1, TPH2, GSK3A, |
| | BDNF |
| nrg1, erbb4, cplx1, tph2, gsk3a, disc1 | NRG1, ErbB4, CPLX1, TPH2, GSK3A, |
| | DISC1 |
| nrg1, erbb4, cplx1, tph2, gsk3b, nrxn1 | NRG1, ErbB4, CPLX1, TPH2, GSK3B, |
| | NRXN1 |
| nrg1, erbb4, cplx1, tph2, gsk3b, bdnf | NRG1, ErbB4, CPLX1, TPH2, GSK3B, |
| | BDNF |
| nrg1, erbb4, cplx1, tph2, gsk3b, disc1 | NRG1, ErbB4, CPLX1, TPH2, GSK3B, |
| | DISC1 |
| nrg1, erbb4, cplx1, tph2, nrxn1, bdnf | NRG1, ErbB4, CPLX1, TPH2, NRXN1, |
| | BDNF |
| nrg1, erbb4, cplx1, tph2, nrxn1, disc1 | NRG1, ErbB4, CPLX1, TPH2, NRXN1, |
| | DISC1 |
| nrg1, erbb4, cplx1, tph2, bdnf, disc1 | NRG1, ErbB4, CPLX1, TPH2, BDNF, |
| | DISC1 |
| nrg1, erbb4, cplx1, gsk3a, gsk3b, nrxn1 | NRG1, ErbB4, CPLX1, GSK3A, GSK3B, |
| | NRXN1 |
| nrg1, erbb4, cplx1, gsk3a, gsk3b, bdnf | NRG1, ErbB4, CPLX1, GSK3A, GSK3B, |
| | BDNF |
| nrg1, erbb4, cplx1, gsk3a, gsk3b, disc1 | NRG1, ErbB4, CPLX1, GSK3A, GSK3B, |
| | DISC1 |
| nrg1, erbb4, cplx1, gsk3a, nrxn1, bdnf | NRG1, ErbB4, CPLX1, GSK3A, NRXN1, |
| | BDNF |
| nrg1, erbb4, cplx1, gsk3a, nrxn1, disc1 | NRG1, ErbB4, CPLX1, GSK3A, NRXN1, |
| | DISC1 |
| nrg1, erbb4, cplx1, gsk3a, bdnf, disc1 | NRG1, ErbB4, CPLX1, GSK3A, BDNF, |
| | DISC1 |
| nrg1, erbb4, cplx1, gsk3b, nrxn1, bdnf | NRG1, ErbB4, CPLX1, GSK3B, NRXN1, |
| | BDNF |
| nrg1, erbb4, cplx1, gsk3b, nrxn1, disc1 | NRG1, ErbB4, CPLX1, GSK3B, NRXN1, |
| | DISC1 |
| nrg1, erbb4, cplx1, gsk3b, bdnf, disc1 | NRG1, ErbB4, CPLX1, GSK3B, BDNF, |
| | DISC1 |
| nrg1, erbb4, cplx1, nrxn1, bdnf, disc1 | NRG1, ErbB4, CPLX1, NRXN1, BDNF, |
| | DISC1 |
| nrg1, erbb4, tph1, tph2, gsk3a, gsk3b | NRG1, ErbB4, TPH1, TPH2, GSK3A, |
| | GSK3B |
| nrg1, erbb4, tph1, tph2, gsk3a, nrxn1 | NRG1, ErbB4, TPH1, TPH2, GSK3A, |
| | NRXN1 |
| nrg1, erbb4, tph1, tph2, gsk3a, bdnf | NRG1, ErbB4, TPH1, TPH2, GSK3A, |
| | BDNF |
| nrg1, erbb4, tph1, tph2, gsk3a, disc1 | NRG1, ErbB4, TPH1, TPH2, GSK3A, |
| | DISC1 |
| nrg1, erbb4, tph1, tph2, gsk3b, nrxn1 | NRG1, ErbB4, TPH1, TPH2, GSK3B, |
| | NRXN1 |
| nrg1, erbb4, tph1, tph2, gsk3b, bdnf | NRG1, ErbB4, TPH1, TPH2, GSK3B, |
| | BDNF |
| nrg1, erbb4, tph1, tph2, gsk3b, disc1 | NRG1, ErbB4, TPH1, TPH2, GSK3B, |
| | DISC1 |
| nrg1, erbb4, tph1, tph2, nrxn1, bdnf | NRG1, ErbB4, TPH1, TPH2, NRXN1, |
| | BDNF |
| nrg1, erbb4, tph1, tph2, nrxn1, disc1 | NRG1, ErbB4, TPH1, TPH2, NRXN1, |
| | DISC1 |
| nrg1, erbb4, tph1, tph2, bdnf, disc1 | NRG1, ErbB4, TPH1, TPH2, BDNF, |
| | DISC1 |
| nrg1, erbb4, tph1, gsk3a, gsk3b, nrxn1 | NRG1, ErbB4, TPH1, GSK3A, GSK3B, |
| | NRXN1 |
| nrg1, erbb4, tph1, gsk3a, gsk3b, bdnf | NRG1, ErbB4, TPH1, GSK3A, GSK3B, |
| | BDNF |
| nrg1, erbb4, tph1, gsk3a, gsk3b, disc1 | NRG1, ErbB4, TPH1, GSK3A, GSK3B, |
| | DISC1 |
| nrg1, erbb4, tph1, gsk3a, nrxn1, bdnf | NRG1, ErbB4, TPH1, GSK3A, NRXN1, |
| | BDNF |
| nrg1, erbb4, tph1, gsk3a, nrxn1, disc1 | NRG1, ErbB4, TPH1, GSK3A, NRXN1, |
| | DISC1 |
| nrg1, erbb4, tph1, gsk3a, bdnf, disc1 | NRG1, ErbB4, TPH1, GSK3A, BDNF, |
| | DISC1 |
| nrg1, erbb4, tph1, gsk3b, nrxn1, bdnf | NRG1, ErbB4, TPH1, GSK3B, NRXN1, |
| | BDNF |
| nrg1, erbb4, tph1, gsk3b, nrxn1, disc1 | NRG1, ErbB4, TPH1, GSK3B, NRXN1, |
| | DISC1 |
| nrg1, erbb4, tph1, gsk3b, bdnf, disc1 | NRG1, ErbB4, TPH1, GSK3B, BDNF, |
| | DISC1 |
| nrg1, erbb4, tph1, nrxn1, bdnf, disc1 | NRG1, ErbB4, TPH1, NRXN1, BDNF, |
| | DISC1 |
| nrg1, erbb4, tph2, gsk3a, gsk3b, nrxn1 | NRG1, ErbB4, TPH2, GSK3A, GSK3B, |
| | NRXN1 |
| nrg1, erbb4, tph2, gsk3a, gsk3b, bdnf | NRG1, ErbB4, TPH2, GSK3A, GSK3B, |
| | BDNF |
| nrg1, erbb4, tph2, gsk3a, gsk3b, disc1 | NRG1, ErbB4, TPH2, GSK3A, GSK3B, |
| | DISC1 |
| nrg1, erbb4, tph2, gsk3a, nrxn1, bdnf | NRG1, ErbB4, TPH2, GSK3A, NRXN1, |
| | BDNF |
| nrg1, erbb4, tph2, gsk3a, nrxn1, disc1 | NRG1, ErbB4, TPH2, GSK3A, NRXN1, |
| | DISC1 |
| nrg1, erbb4, tph2, gsk3a, bdnf, disc1 | NRG1, ErbB4, TPH2, GSK3A, BDNF, |
| | DISC1 |
| nrg1, erbb4, tph2, gsk3b, nrxn1, bdnf | NRG1, ErbB4, TPH2, GSK3B, NRXN1, |
| | BDNF |
| nrg1, erbb4, tph2, gsk3b, nrxn1, disc1 | NRG1, ErbB4, TPH2, GSK3B, NRXN1, |
| | DISC1 |
| nrg1, erbb4, tph2, gsk3b, bdnf, disc1 | NRG1, ErbB4, TPH2, GSK3B, BDNF, |
| | DISC1 |
| nrg1, erbb4, tph2, nrxn1, bdnf, disc1 | NRG1, ErbB4, TPH2, NRXN1, BDNF, |
| | DISC1 |
| nrg1, erbb4, gsk3a, gsk3b, nrxn1, bdnf | NRG1, ErbB4, GSK3A, GSK3B, NRXN1, |
| | BDNF |
| nrg1, erbb4, gsk3a, gsk3b, nrxn1, disc1 | NRG1, ErbB4, GSK3A, GSK3B, NRXN1, |
| | DISC1 |
| nrg1, erbb4, gsk3a, gsk3b, bdnf, disc1 | NRG1, ErbB4, GSK3A, GSK3B, BDNF, |
| | DISC1 |
| nrg1, erbb4, gsk3a, nrxn1, bdnf, disc1 | NRG1, ErbB4, GSK3A, NRXN1, BDNF, |
| | DISC1 |
| nrg1, erbb4, gsk3b, nrxn1, bdnf, disc1 | NRG1, ErbB4, GSK3B, NRXN1, BDNF, |
| | DISC1 |
| nrg1, cplx1, tph1, tph2, gsk3a, gsk3b | NRG1, CPLX1, TPH1, TPH2, GSK3A, |
| | GSK3B |
| nrg1, cplx1, tph1, tph2, gsk3a, nrxn1 | NRG1, CPLX1, TPH1, TPH2, GSK3A, |
| | NRXN1 |
| nrg1, cplx1, tph1, tph2, gsk3a, bdnf | NRG1, CPLX1, TPH1, TPH2, GSK3A, |
| | BDNF |
| nrg1, cplx1, tph1, tph2, gsk3a, disc1 | NRG1, CPLX1, TPH1, TPH2, GSK3A, |
| | DISC1 |
| nrg1, cplx1, tph1, tph2, gsk3b, nrxn1 | NRG1, CPLX1, TPH1, TPH2, GSK3B, |
| | NRXN1 |
| nrg1, cplx1, tph1, tph2, gsk3b, bdnf | NRG1, CPLX1, TPH1, TPH2, GSK3B, |
| | BDNF |
| nrg1, cplx1, tph1, tph2, gsk3b, disc1 | NRG1, CPLX1, TPH1, TPH2, GSK3B, |
| | DISC1 |
| nrg1, cplx1, tph1, tph2, nrxn1, bdnf | NRG1, CPLX1, TPH1, TPH2, NRXN1, |
| | BDNF |
| nrg1, cplx1, tph1, tph2, nrxn1, disc1 | NRG1, CPLX1, TPH1, TPH2, NRXN1, |
| | DISC1 |
| nrg1, cplx1, tph1, tph2, bdnf, disc1 | NRG1, CPLX1, TPH1, TPH2, BDNF, |
| | DISC1 |
| nrg1, cplx1, tph1, gsk3a, gsk3b, nrxn1 | NRG1, CPLX1, TPH1, GSK3A, GSK3B, |
| | NRXN1 |
| nrg1, cplx1, tph1, gsk3a, gsk3b, bdnf | NRG1, CPLX1, TPH1, GSK3A, GSK3B, |
| | BDNF |
| nrg1, cplx1, tph1, gsk3a, gsk3b, disc1 | NRG1, CPLX1, TPH1, GSK3A, GSK3B, |
| | DISC1 |
| nrg1, cplx1, tph1, gsk3a, nrxn1, bdnf | NRG1, CPLX1, TPH1, GSK3A, NRXN1, |
| | BDNF |
| nrg1, cplx1, tph1, gsk3a, nrxn1, disc1 | NRG1, CPLX1, TPH1, GSK3A, NRXN1, |
| | DISC1 |
| nrg1, cplx1, tph1, gsk3a, bdnf, disc1 | NRG1, CPLX1, TPH1, GSK3A, BDNF, |
| | DISC1 |
| nrg1, cplx1, tph1, gsk3b, nrxn1, bdnf | NRG1, CPLX1, TPH1, GSK3B, NRXN1, |
| | BDNF |
| nrg1, cplx1, tph1, gsk3b, nrxn1, disc1 | NRG1, CPLX1, TPH1, GSK3B, NRXN1, |
| | DISC1 |
| nrg1, cplx1, tph1, gsk3b, bdnf, disc1 | NRG1, CPLX1, TPH1, GSK3B, BDNF, |
| | DISC1 |
| nrg1, cplx1, tph1, nrxn1, bdnf, disc1 | NRG1, CPLX1, TPH1, NRXN1, BDNF, |
| | DISC1 |
| nrg1, cplx1, tph2, gsk3a, gsk3b, nrxn1 | NRG1, CPLX1, TPH2, GSK3A, GSK3B, |
| | NRXN1 |
| nrg1, cplx1, tph2, gsk3a, gsk3b, bdnf | NRG1, CPLX1, TPH2, GSK3A, GSK3B, |
| | BDNF |
| nrg1, cplx1, tph2, gsk3a, gsk3b, disc1 | NRG1, CPLX1, TPH2, GSK3A, GSK3B, |
| | DISC1 |
| nrg1, cplx1, tph2, gsk3a, nrxn1, bdnf | NRG1, CPLX1, TPH2, GSK3A, NRXN1, |
| | BDNF |
| nrg1, cplx1, tph2, gsk3a, nrxn1, disc1 | NRG1, CPLX1, TPH2, GSK3A, NRXN1, |
| | DISC1 |
| nrg1, cplx1, tph2, gsk3a, bdnf, disc1 | NRG1, CPLX1, TPH2, GSK3A, BDNF, |
| | DISC1 |
| nrg1, cplx1, tph2, gsk3b, nrxn1, bdnf | NRG1, CPLX1, TPH2, GSK3B, NRXN1, |
| | BDNF |
| nrg1, cplx1, tph2, gsk3b, nrxn1, disc1 | NRG1, CPLX1, TPH2, GSK3B, NRXN1, |
| | DISC1 |
| nrg1, cplx1, tph2, gsk3b, bdnf, disc1 | NRG1, CPLX1, TPH2, GSK3B, BDNF, |
| | DISC1 |
| nrg1, cplx1, tph2, nrxn1, bdnf, disc1 | NRG1, CPLX1, TPH2, NRXN1, BDNF, |
| | DISC1 |
| nrg1, cplx1, gsk3a, gsk3b, nrxn1, bdnf | NRG1, CPLX1, GSK3A, GSK3B, NRXN1, |
| | BDNF |
| nrg1, cplx1, gsk3a, gsk3b, nrxn1, disc1 | NRG1, CPLX1, GSK3A, GSK3B, NRXN1, |
| | DISC1 |
| nrg1, cplx1, gsk3a, gsk3b, bdnf, disc1 | NRG1, CPLX1, GSK3A, GSK3B, BDNF, |
| | DISC1 |
| nrg1, cplx1, gsk3a, nrxn1, bdnf, disc1 | NRG1, CPLX1, GSK3A, NRXN1, BDNF, |
| | DISC1 |
| nrg1, cplx1, gsk3b, nrxn1, bdnf, disc1 | NRG1, CPLX1, GSK3B, NRXN1, BDNF, |
| | DISC1 |
| nrg1, tph1, tph2, gsk3a, gsk3b, nrxn1 | NRG1, TPH1, TPH2, GSK3A, GSK3B, |
| | NRXN1 |
| nrg1, tph1, tph2, gsk3a, gsk3b, bdnf | NRG1, TPH1, TPH2, GSK3A, GSK3B, |
| | BDNF |
| nrg1, tph1, tph2, gsk3a, gsk3b, disc1 | NRG1, TPH1, TPH2, GSK3A, GSK3B, |
| | DISC1 |
| nrg1, tph1, tph2, gsk3a, nrxn1, bdnf | NRG1, TPH1, TPH2, GSK3A, NRXN1, |
| | BDNF |
| nrg1, tph1, tph2, gsk3a, nrxn1, disc1 | NRG1, TPH1, TPH2, GSK3A, NRXN1, |
| | DISC1 |
| nrg1, tph1, tph2, gsk3a, bdnf, disc1 | NRG1, TPH1, TPH2, GSK3A, BDNF, |
| | DISC1 |
| nrg1, tph1, tph2, gsk3b, nrxn1, bdnf | NRG1, TPH1, TPH2, GSK3B, NRXN1, |
| | BDNF |
| nrg1, tph1, tph2, gsk3b, nrxn1, disc1 | NRG1, TPH1, TPH2, GSK3B, NRXN1, |
| | DISC1 |
| nrg1, tph1, tph2, gsk3b, bdnf, disc1 | NRG1, TPH1, TPH2, GSK3B, BDNF, |
| | DISC1 |
| nrg1, tph1, tph2, nrxn1, bdnf, disc1 | NRG1, TPH1, TPH2, NRXN1, BDNF, |
| | DISC1 |
| nrg1, tph1, gsk3a, gsk3b, nrxn1, bdnf | NRG1, TPH1, GSK3A, GSK3B, NRXN1, |
| | BDNF |
| nrg1, tph1, gsk3a, gsk3b, nrxn1, disc1 | NRG1, TPH1, GSK3A, GSK3B, NRXN1, |
| | DISC1 |
| nrg1, tph1, gsk3a, gsk3b, bdnf, disc1 | NRG1, TPH1, GSK3A, GSK3B, BDNF, |
| | DISC1 |
| nrg1, tph1, gsk3a, nrxn1, bdnf, disc1 | NRG1, TPH1, GSK3A, NRXN1, BDNF, |
| | DISC1 |
| nrg1, tph1, gsk3b, nrxn1, bdnf, disc1 | NRG1, TPH1, GSK3B, NRXN1, BDNF, |
| | DISC1 |
| nrg1, tph2, gsk3a, gsk3b, nrxn1, bdnf | NRG1, TPH2, GSK3A, GSK3B, NRXN1, |
| | BDNF |
| nrg1, tph2, gsk3a, gsk3b, nrxn1, disc1 | NRG1, TPH2, GSK3A, GSK3B, NRXN1, |
| | DISC1 |
| nrg1, tph2, gsk3a, gsk3b, bdnf, disc1 | NRG1, TPH2, GSK3A, GSK3B, BDNF, |
| | DISC1 |
| nrg1, tph2, gsk3a, nrxn1, bdnf, disc1 | NRG1, TPH2, GSK3A, NRXN1, BDNF, |
| | DISC1 |
| nrg1, tph2, gsk3b, nrxn1, bdnf, disc1 | NRG1, TPH2, GSK3B, NRXN1, BDNF, |
| | DISC1 |
| nrg1, gsk3a, gsk3b, nrxn1, bdnf, disc1 | NRG1, GSK3A, GSK3B, NRXN1, BDNF, |
| | DISC1 |
| erbb4, cplx1, tph1, tph2, gsk3a, gsk3b | ErbB4, CPLX1, TPH1, TPH2, GSK3A, |
| | GSK3B |
| erbb4, cplx1, tph1, tph2, gsk3a, nrxn1 | ErbB4, CPLX1, TPH1, TPH2, GSK3A, |
| | NRXN1 |
| erbb4, cplx1, tph1, tph2, gsk3a, bdnf | ErbB4, CPLX1, TPH1, TPH2, GSK3A, |
| | BDNF |
| erbb4, cplx1, tph1, tph2, gsk3a, disc1 | ErbB4, CPLX1, TPH1, TPH2, GSK3A, |
| | DISC1 |
| erbb4, cplx1, tph1, tph2, gsk3b, nrxn1 | ErbB4, CPLX1, TPH1, TPH2, GSK3B, |
| | NRXN1 |
| erbb4, cplx1, tph1, tph2, gsk3b, bdnf | ErbB4, CPLX1, TPH1, TPH2, GSK3B, |
| | BDNF |
| erbb4, cplx1, tph1, tph2, gsk3b, disc1 | ErbB4, CPLX1, TPH1, TPH2, GSK3B, |
| | DISC1 |
| erbb4, cplx1, tph1, tph2, nrxn1, bdnf | ErbB4, CPLX1, TPH1, TPH2, NRXN1, |
| | BDNF |
| erbb4, cplx1, tph1, tph2, nrxn1, disc1 | ErbB4, CPLX1, TPH1, TPH2, NRXN1, |
| | DISC1 |
| erbb4, cplx1, tph1, tph2, bdnf, disc1 | ErbB4, CPLX1, TPH1, TPH2, BDNF, |
| | DISC1 |
| erbb4, cplx1, tph1, gsk3a, gsk3b, nrxn1 | ErbB4, CPLX1, TPH1, GSK3A, GSK3B, |
| | NRXN1 |
| erbb4, cplx1, tph1, gsk3a, gsk3b, bdnf | ErbB4, CPLX1, TPH1, GSK3A, GSK3B, |
| | BDNF |
| erbb4, cplx1, tph1, gsk3a, gsk3b, disc1 | ErbB4, CPLX1, TPH1, GSK3A, GSK3B, |
| | DISC1 |
| erbb4, cplx1, tph1, gsk3a, nrxn1, bdnf | ErbB4, CPLX1, TPH1, GSK3A, NRXN1, |
| | BDNF |
| erbb4, cplx1, tph1, gsk3a, nrxn1, disc1 | ErbB4, CPLX1, TPH1, GSK3A, NRXN1, |
| | DISC1 |
| erbb4, cplx1, tph1, gsk3a, bdnf, disc1 | ErbB4, CPLX1, TPH1, GSK3A, BDNF, |
| | DISC1 |
| erbb4, cplx1, tph1, gsk3b, nrxn1, bdnf | ErbB4, CPLX1, TPH1, GSK3B, NRXN1, |
| | BDNF |
| erbb4, cplx1, tph1, gsk3b, nrxn1, disc1 | ErbB4, CPLX1, TPH1, GSK3B, NRXN1, |
| | DISC1 |
| erbb4, cplx1, tph1, gsk3b, bdnf, disc1 | ErbB4, CPLX1, TPH1, GSK3B, BDNF, |
| | DISC1 |
| erbb4, cplx1, tph1, nrxn1, bdnf, disc1 | ErbB4, CPLX1, TPH1, NRXN1, BDNF, |
| | DISC1 |
| erbb4, cplx1, tph2, gsk3a, gsk3b, nrxn1 | ErbB4, CPLX1, TPH2, GSK3A, GSK3B, |
| | NRXN1 |
| erbb4, cplx1, tph2, gsk3a, gsk3b, bdnf | ErbB4, CPLX1, TPH2, GSK3A, GSK3B, |
| | BDNF |
| erbb4, cplx1, tph2, gsk3a, gsk3b, disc1 | ErbB4, CPLX1, TPH2, GSK3A, GSK3B, |
| | DISC1 |
| erbb4, cplx1, tph2, gsk3a, nrxn1, bdnf | ErbB4, CPLX1, TPH2, GSK3A, NRXN1, |
| | BDNF |
| erbb4, cplx1, tph2, gsk3a, nrxn1, disc1 | ErbB4, CPLX1, TPH2, GSK3A, NRXN1, |
| | DISC1 |
| erbb4, cplx1, tph2, gsk3a, bdnf, disc1 | ErbB4, CPLX1, TPH2, GSK3A, BDNF, |
| | DISC1 |
| erbb4, cplx1, tph2, gsk3b, nrxn1, bdnf | ErbB4, CPLX1, TPH2, GSK3B, NRXN1, |
| | BDNF |
| erbb4, cplx1, tph2, gsk3b, nrxn1, disc1 | ErbB4, CPLX1, TPH2, GSK3B, NRXN1, |
| | DISC1 |
| erbb4, cplx1, tph2, gsk3b, bdnf, disc1 | ErbB4, CPLX1, TPH2, GSK3B, BDNF, |
| | DISC1 |
| erbb4, cplx1, tph2, nrxn1, bdnf, disc1 | ErbB4, CPLX1, TPH2, NRXN1, BDNF, |
| | DISC1 |
| erbb4, cplx1, gsk3a, gsk3b, nrxn1, bdnf | ErbB4, CPLX1, GSK3A, GSK3B, NRXN1, |
| | BDNF |
| erbb4, cplx1, gsk3a, gsk3b, nrxn1, disc1 | ErbB4, CPLX1, GSK3A, GSK3B, NRXN1, |
| | DISC1 |
| erbb4, cplx1, gsk3a, gsk3b, bdnf, disc1 | ErbB4, CPLX1, GSK3A, GSK3B, BDNF, |
| | DISC1 |
| erbb4, cplx1, gsk3a, nrxn1, bdnf, disc1 | ErbB4, CPLX1, GSK3A, NRXN1, BDNF, |
| | DISC1 |
| erbb4, cplx1, gsk3b, nrxn1, bdnf, disc1 | ErbB4, CPLX1, GSK3B, NRXN1, BDNF, |
| | DISC1 |
| erbb4, tph1, tph2, gsk3a, gsk3b, nrxn1 | ErbB4, TPH1, TPH2, GSK3A, GSK3B, |
| | NRXN1 |
| erbb4, tph1, tph2, gsk3a, gsk3b, bdnf | ErbB4, TPH1, TPH2, GSK3A, GSK3B, |
| | BDNF |
| erbb4, tph1, tph2, gsk3a, gsk3b, disc1 | ErbB4, TPH1, TPH2, GSK3A, GSK3B, |
| | DISC1 |
| erbb4, tph1, tph2, gsk3a, nrxn1, bdnf | ErbB4, TPH1, TPH2, GSK3A, NRXN1, |
| | BDNF |
| erbb4, tph1, tph2, gsk3a, nrxn1, disc1 | ErbB4, TPH1, TPH2, GSK3A, NRXN1, |
| | DISC1 |
| erbb4, tph1, tph2, gsk3a, bdnf, disc1 | ErbB4, TPH1, TPH2, GSK3A, BDNF, |
| | DISC1 |
| erbb4, tph1, tph2, gsk3b, nrxn1, bdnf | ErbB4, TPH1, TPH2, GSK3B, NRXN1, |
| | BDNF |
| erbb4, tph1, tph2, gsk3b, nrxn1, disc1 | ErbB4, TPH1, TPH2, GSK3B, NRXN1, |
| | DISC1 |
| erbb4, tph1, tph2, gsk3b, bdnf, disc1 | ErbB4, TPH1, TPH2, GSK3B, BDNF, |
| | DISC1 |
| erbb4, tph1, tph2, nrxn1, bdnf, disc1 | ErbB4, TPH1, TPH2, NRXN1, BDNF, |
| | DISC1 |
| erbb4, tph1, gsk3a, gsk3b, nrxn1, bdnf | ErbB4, TPH1, GSK3A, GSK3B, NRXN1, |
| | BDNF |
| erbb4, tph1, gsk3a, gsk3b, nrxn1, disc1 | ErbB4, TPH1, GSK3A, GSK3B, NRXN1, |
| | DISC1 |
| erbb4, tph1, gsk3a, gsk3b, bdnf, disc1 | ErbB4, TPH1, GSK3A, GSK3B, BDNF, |
| | DISC1 |
| erbb4, tph1, gsk3a, nrxn1, bdnf, disc1 | ErbB4, TPH1, GSK3A, NRXN1, BDNF, |
| | DISC1 |
| erbb4, tph1, gsk3b, nrxn1, bdnf, disc1 | ErbB4, TPH1, GSK3B, NRXN1, BDNF, |
| | DISC1 |
| erbb4, tph2, gsk3a, gsk3b, nrxn1, bdnf | ErbB4, TPH2, GSK3A, GSK3B, NRXN1, |
| | BDNF |
| erbb4, tph2, gsk3a, gsk3b, nrxn1, disc1 | ErbB4, TPH2, GSK3A, GSK3B, NRXN1, |
| | DISC1 |
| erbb4, tph2, gsk3a, gsk3b, bdnf, disc1 | ErbB4, TPH2, GSK3A, GSK3B, BDNF, |
| | DISC1 |
| erbb4, tph2, gsk3a, nrxn1, bdnf, disc1 | ErbB4, TPH2, GSK3A, NRXN1, BDNF, |
| | DISC1 |
| erbb4, tph2, gsk3b, nrxn1, bdnf, disc1 | ErbB4, TPH2, GSK3B, NRXN1, BDNF, |
| | DISC1 |
| erbb4, gsk3a, gsk3b, nrxn1, bdnf, disc1 | ErbB4, GSK3A, GSK3B, NRXN1, BDNF, |
| | DISC1 |
| cplx1, tph1, tph2, gsk3a, gsk3b, nrxn1 | CPLX1, TPH1, TPH2, GSK3A, GSK3B, |
| | NRXN1 |
| cplx1, tph1, tph2, gsk3a, gsk3b, bdnf | CPLX1, TPH1, TPH2, GSK3A, GSK3B, |
| | BDNF |
| cplx1, tph1, tph2, gsk3a, gsk3b, disc1 | CPLX1, TPH1, TPH2, GSK3A, GSK3B, |
| | DISC1 |
| cplx1, tph1, tph2, gsk3a, nrxn1, bdnf | CPLX1, TPH1, TPH2, GSK3A, NRXN1, |
| | BDNF |
| cplx1, tph1, tph2, gsk3a, nrxn1, disc1 | CPLX1, TPH1, TPH2, GSK3A, NRXN1, |
| | DISC1 |
| cplx1, tph1, tph2, gsk3a, bdnf, disc1 | CPLX1, TPH1, TPH2, GSK3A, BDNF, |
| | DISC1 |
| cplx1, tph1, tph2, gsk3b, nrxn1, bdnf | CPLX1, TPH1, TPH2, GSK3B, NRXN1, |
| | BDNF |
| cplx1, tph1, tph2, gsk3b, nrxn1, disc1 | CPLX1, TPH1, TPH2, GSK3B, NRXN1, |
| | DISC1 |
| cplx1, tph1, tph2, gsk3b, bdnf, disc1 | CPLX1, TPH1, TPH2, GSK3B, BDNF, |
| | DISC1 |
| cplx1, tph1, tph2, nrxn1, bdnf, disc1 | CPLX1, TPH1, TPH2, NRXN1, BDNF, |
| | DISC1 |
| cplx1, tph1, gsk3a, gsk3b, nrxn1, bdnf | CPLX1, TPH1, GSK3A, GSK3B, NRXN1, |
| | BDNF |
| cplx1, tph1, gsk3a, gsk3b, nrxn1, disc1 | CPLX1, TPH1, GSK3A, GSK3B, NRXN1, |
| | DISC1 |
| cplx1, tph1, gsk3a, gsk3b, bdnf, disc1 | CPLX1, TPH1, GSK3A, GSK3B, BDNF, |
| | DISC1 |
| cplx1, tph1, gsk3a, nrxn1, bdnf, disc1 | CPLX1, TPH1, GSK3A, NRXN1, BDNF, |
| | DISC1 |
| cplx1, tph1, gsk3b, nrxn1, bdnf, disc1 | CPLX1, TPH1, GSK3B, NRXN1, BDNF, |
| | DISC1 |
| cplx1, tph2, gsk3a, gsk3b, nrxn1, bdnf | CPLX1, TPH2, GSK3A, GSK3B, NRXN1, |
| | BDNF |
| cplx1, tph2, gsk3a, gsk3b, nrxn1, disc1 | CPLX1, TPH2, GSK3A, GSK3B, NRXN1, |
| | DISC1 |
| cplx1, tph2, gsk3a, gsk3b, bdnf, disc1 | CPLX1, TPH2, GSK3A, GSK3B, BDNF, |
| | DISC1 |
| cplx1, tph2, gsk3a, nrxn1, bdnf, disc1 | CPLX1, TPH2, GSK3A, NRXN1, BDNF, |
| | DISC1 |
| cplx1, tph2, gsk3b, nrxn1, bdnf, disc1 | CPLX1, TPH2, GSK3B, NRXN1, BDNF, |
| | DISC1 |
| cplx1, gsk3a, gsk3b, nrxn1, bdnf, disc1 | CPLX1, GSK3A, GSK3B, NRXN1, BDNF, |
| | DISC1 |
| tph1, tph2, gsk3a, gsk3b, nrxn1, bdnf | TPH1, TPH2, GSK3A, GSK3B, NRXN1, |
| | BDNF |
| tph1, tph2, gsk3a, gsk3b, nrxn1, disc1 | TPH1, TPH2, GSK3A, GSK3B, NRXN1, |
| | DISC1 |
| tph1, tph2, gsk3a, gsk3b, bdnf, disc1 | TPH1, TPH2, GSK3A, GSK3B, BDNF, |
| | DISC1 |
| tph1, tph2, gsk3a, nrxn1, bdnf, disc1 | TPH1, TPH2, GSK3A, NRXN1, BDNF, |
| | DISC1 |
| tph1, tph2, gsk3b, nrxn1, bdnf, disc1 | TPH1, TPH2, GSK3B, NRXN1, BDNF, |
| | DISC1 |
| tph1, gsk3a, gsk3b, nrxn1, bdnf, disc1 | TPH1, GSK3A, GSK3B, NRXN1, BDNF, |
| | DISC1 |
| tph2, gsk3a, gsk3b, nrxn1, bdnf, disc1 | TPH2, GSK3A, GSK3B, NRXN1, BDNF, |
| | DISC1 |
| nrg1, erbb4, cplx1, tph1, tph2, gsk3a, | NRG1, ErbB4, CPLX1, TPH1, TPH2, |
| gsk3b | GSK3A, GSK3B |
| nrg1, erbb4, cplx1, tph1, tph2, gsk3a, | NRG1, ErbB4, CPLX1, TPH1, TPH2, |
| nrxn1 | GSK3A, NRXN1 |
| nrg1, erbb4, cplx1, tph1, tph2, gsk3a, bdnf | NRG1, ErbB4, CPLX1, TPH1, TPH2, |
| | GSK3A, BDNF |
| nrg1, erbb4, cplx1, tph1, tph2, gsk3a, | NRG1, ErbB4, CPLX1, TPH1, TPH2, |
| disc1 | GSK3A, DISC1 |
| nrg1, erbb4, cplx1, tph1, tph2, gsk3b, | NRG1, ErbB4, CPLX1, TPH1, TPH2, |
| nrxn1 | GSK3B, NRXN1 |
| nrg1, erbb4, cplx1, tph1, tph2, gsk3b, bdnf | NRG1, ErbB4, CPLX1, TPH1, TPH2, |
| | GSK3B, BDNF |
| nrg1, erbb4, cplx1, tph1, tph2, gsk3b, | NRG1, ErbB4, CPLX1, TPH1, TPH2, |
| disc1 | GSK3B, DISC1 |
| nrg1, erbb4, cplx1, tph1, tph2, nrxn1, bdnf | NRG1, ErbB4, CPLX1, TPH1, TPH2, |
| | NRXN1, BDNF |
| nrg1, erbb4, cplx1, tph1, tph2, nrxn1, | NRG1, ErbB4, CPLX1, TPH1, TPH2, |
| disc1 | NRXN1, DISC1 |
| nrg1, erbb4, cplx1, tph1, tph2, bdnf, disc1 | NRG1, ErbB4, CPLX1, TPH1, TPH2, |
| | BDNF, DISC1 |
| nrg1, erbb4, cplx1, tph1, gsk3a, gsk3b, | NRG1, ErbB4, CPLX1, TPH1, GSK3A, |
| nrxn1 | GSK3B, NRXN1 |
| nrg1, erbb4, cplx1, tph1, gsk3a, gsk3b, | NRG1, ErbB4, CPLX1, TPH1, GSK3A, |
| bdnf | GSK3B, BDNF |
| nrg1, erbb4, cplx1, tph1, gsk3a, gsk3b, | NRG1, ErbB4, CPLX1, TPH1, GSK3A, |
| disc1 | GSK3B, DISC1 |
| nrg1, erbb4, cplx1, tph1, gsk3a, nrxn1, | NRG1, ErbB4, CPLX1, TPH1, GSK3A, |
| bdnf | NRXN1, BDNF |
| nrg1, erbb4, cplx1, tph1, gsk3a, nrxn1, | NRG1, ErbB4, CPLX1, TPH1, GSK3A, |
| disc1 | NRXN1, DISC1 |
| nrg1, erbb4, cplx1, tph1, gsk3a, bdnf, | NRG1, ErbB4, CPLX1, TPH1, GSK3A, |
| disc1 | BDNF, DISC1 |
| nrg1, erbb4, cplx1, tph1, gsk3b, nrxn1, | NRG1, ErbB4, CPLX1, TPH1, GSK3B, |
| bdnf | NRXN1, BDNF |
| nrg1, erbb4, cplx1, tph1, gsk3b, nrxn1, | NRG1, ErbB4, CPLX1, TPH1, GSK3B, |
| disc1 | NRXN1, DISC1 |
| nrg1, erbb4, cplx1, tph1, gsk3b, bdnf, | NRG1, ErbB4, CPLX1, TPH1, GSK3B, |
| disc1 | BDNF, DISC1 |
| nrg1, erbb4, cplx1, tph1, nrxn1, bdnf, | NRG1, ErbB4, CPLX1, TPH1, NRXN1, |
| disc1 | BDNF, DISC1 |
| nrg1, erbb4, cplx1, tph2, gsk3a, gsk3b, | NRG1, ErbB4, CPLX1, TPH2, GSK3A, |
| nrxn1 | GSK3B, NRXN1 |
| nrg1, erbb4, cplx1, tph2, gsk3a, gsk3b, | NRG1, ErbB4, CPLX1, TPH2, GSK3A, |
| bdnf | GSK3B, BDNF |
| nrg1, erbb4, cplx1, tph2, gsk3a, gsk3b, | NRG1, ErbB4, CPLX1, TPH2, GSK3A, |
| disc1 | GSK3B, DISC1 |
| nrg1, erbb4, cplx1, tph2, gsk3a, nrxn1, | NRG1, ErbB4, CPLX1, TPH2, GSK3A, |
| bdnf | NRXN1, BDNF |
| nrg1, erbb4, cplx1, tph2, gsk3a, nrxn1, | NRG1, ErbB4, CPLX1, TPH2, GSK3A, |
| disc1 | NRXN1, DISC1 |
| nrg1, erbb4, cplx1, tph2, gsk3a, bdnf, | NRG1, ErbB4, CPLX1, TPH2, GSK3A, |
| disc1 | BDNF, DISC1 |
| nrg1, erbb4, cplx1, tph2, gsk3b, nrxn1, | NRG1 ErbB4, CPLX1, TPH2, GSK3B, |
| bdnf | NRXN1, BDNF |
| nrg1, erbb4, cplx1, tph2, gsk3b, nrxn1, | NRG1 ErbB4, CPLX1, TPH2, GSK3B, |
| disc1 | NRXN1, DISC1 |
| nrg1, erbb4, cplx1, tph2, gsk3b, bdnf, | NRG1, ErbB4, CPLX1, TPH2, GSK3B, |
| disc1 | BDNF, DISC1 |
| nrg1, erbb4, cplx1, tph2, nrxn1, bdnf, | NRG1, ErbB4, CPLX1, TPH2, NRXN1, |
| disc1 | BDNF, DISC1 |
| nrg1, erbb4, cplx1, gsk3a, gsk3b, nrxn1, | NRG1, ErbB4, CPLX1, GSK3A, GSK3B, |
| bdnf | NRXN1, BDNF |
| nrg1, erbb4, cplx1, gsk3a, gsk3b, nrxn1, | NRG1, ErbB4, CPLX1, GSK3A, GSK3B, |
| disc1 | NRXN1, DISC1 |
| nrg1, erbb4, cplx1, gsk3a, gsk3b, bdnf, | NRG1, ErbB4, CPLX1, GSK3A, GSK3B, |
| disc1 | BDNF, DISC1 |
| nrg1, erbb4, cplx1, gsk3a, nrxn1, bdnf, | NRG1, ErbB4, CPLX1, GSK3A, NRXN1, |
| disc1 | BDNF, DISC1 |
| nrg1, erbb4, cplx1, gsk3b, nrxn1, bdnf, | NRG1, ErbB4, CPLX1, GSK3B, NRXN1, |
| disc1 | BDNF, DISC1 |
| nrg1, erbb4, tph1, tph2, gsk3a, gsk3b, | NRG1, ErbB4, TPH1, TPH2, GSK3A, |
| nrxn1 | GSK3B, NRXN1 |
| nrg1, erbb4, tph1, tph2, gsk3a, gsk3b, | NRG1, ErbB4, TPH1, TPH2, GSK3A, |
| bdnf | GSK3B, BDNF |
| nrg1, erbb4, tph1, tph2, gsk3a, gsk3b, | NRG1, ErbB4, TPH1, TPH2, GSK3A, |
| disc1 | GSK3B, DISC1 |
| nrg1, erbb4, tph1, tph2, gsk3a, nrxn1, | NRG1, ErbB4, TPH1, TPH2, GSK3A, |
| bdnf | NRXN1, BDNF |
| nrg1, erbb4, tph1, tph2, gsk3a, nrxn1, | NRG1, ErbB4, TPH1, TPH2, GSK3A, |
| disc1 | NRXN1, DISC1 |
| nrg1, erbb4, tph1, tph2, gsk3a, bdnf, disc1 | NRG1, ErbB4, TPH1, TPH2, GSK3A, |
| | BDNF, DISC1 |
| nrg1, erbb4, tph1, tph2, gsk3b, nrxn1, | NRG1, ErbB4, TPH1, TPH2, GSK3B, |
| bdnf | NRXN1, BDNF |
| nrg1, erbb4, tph1, tph2, gsk3b, nrxn1, | NRG1, ErbB4, TPH1, TPH2, GSK3B, |
| disc1 | NRXN1, DISC1 |
| nrg1, erbb4, tph1, tph2, gsk3b, bdnf, disc1 | NRG1, ErbB4, TPH1, TPH2, GSK3B, |
| | BDNF, DISC1 |
| nrg1, erbb4, tph1, tph2, nrxn1, bdnf, disc1 | NRG1, ErbB4, TPH1, TPH2, NRXN1, |
| | BDNF, DISC1 |
| nrg1, erbb4, tph1, gsk3a, gsk3b, nrxn1, | NRG1, ErbB4, TPH1, GSK3A, GSK3B, |
| bdnf | NRXN1, BDNF |
| nrg1, erbb4, tph1, gsk3a, gsk3b, nrxn1, | NRG1, ErbB4, TPH1, GSK3A, GSK3B, |
| disc1 | NRXN1, DISC1 |
| nrg1, erbb4, tph1, gsk3a, gsk3b, bdnf, | NRG1, ErbB4, TPH1, GSK3A, GSK3B, |
| disc1 | BDNF, DISC1 |
| nrg1, erbb4, tph1, gsk3a, nrxn1, bdnf, | NRG1, ErbB4, TPH1, GSK3A, NRXN1, |
| disc1 | BDNF, DISC1 |
| nrg1, erbb4, tph1, gsk3b, nrxn1, bdnf, | NRG1, ErbB4, TPH1, GSK3B, NRXN1, |
| disc1 | BDNF, DISC1 |
| nrg1, erbb4, tph2, gsk3a, gsk3b, nrxn1, | NRG1, ErbB4, TPH2, GSK3A, GSK3B, |
| bdnf | NRXN1, BDNF |
| nrg1, erbb4, tph2, gsk3a, gsk3b, nrxn1, | NRG1, ErbB4, TPH2, GSK3A, GSK3B, |
| disc1 | NRXN1, DISC1 |
| nrg1, erbb4, tph2, gsk3a, gsk3b, bdnf, | NRG1, ErbB4, TPH2, GSK3A, GSK3B, |
| disc1 | BDNF, DISC1 |
| nrg1, erbb4, tph2, gsk3a, nrxn1, bdnf, | NRG1, ErbB4, TPH2, GSK3A, NRXN1, |
| disc1 | BDNF, DISC1 |
| nrg1, erbb4, tph2, gsk3b, nrxn1, bdnf, | NRG1, ErbB4, TPH2, GSK3B, NRXN1, |
| disc1 | BDNF, DISC1 |
| nrg1, erbb4, gsk3a, gsk3b, nrxn1, bdnf, | NRG1, ErbB4, GSK3A, GSK3B, NRXN1, |
| disc1 | BDNF, DISC1 |
| nrg1, cplx1, tph1, tph2, gsk3a, gsk3b, | NRG1, CPLX1, TPH1, TPH2, GSK3A, |
| nrxn1 | GSK3B, NRXN1 |
| nrg1, cplx1, tph1, tph2, gsk3a, gsk3b, bdnf | NRG1, CPLX1, TPH1, TPH2, GSK3A, |
| | GSK3B, BDNF |
| nrg1, cplx1, tph1, tph2, gsk3a, gsk3b, | NRG1, CPLX1, TPH1, TPH2, GSK3A, |
| disc1 | GSK3B, DISC1 |
| nrg1, cplx1, tph1, tph2, gsk3a, nrxn1, bdnf | NRG1, CPLX1, TPH1, TPH2, GSK3A, |
| | NRXN1, BDNF |
| nrg1, cplx1, tph1, tph2, gsk3a, nrxn1, | NRG1, CPLX1, TPH1, TPH2, GSK3A, |
| disc1 | NRXN1, DISC1 |
| nrg1, cplx1, tph1, tph2, gsk3a, bdnf, disc1 | NRG1, CPLX1, TPH1, TPH2, GSK3A, |
| | BDNF, DISC1 |
| nrg1, cplx1, tph1, tph2, gsk3b, nrxn1, bdnf | NRG1, CPLX1, TPH1, TPH2, GSK3B, |
| | NRXN1, BDNF |
| nrg1, cplx1, tph1, tph2, gsk3b, nrxn1, | NRG1, CPLX1, TPH1, TPH2, GSK3B, |
| disc1 | NRXN1, DISC1 |
| nrg1, cplx1, tph1, tph2, gsk3b, bdnf, disc1 | NRG1, CPLX1, TPH1, TPH2, GSK3B, |
| | BDNF, DISC1 |
| nrg1, cplx1, tph1, tph2, nrxn1, bdnf, disc1 | NRG1, CPLX1, TPH1, TPH2, NRXN1, |
| | BDNF, DISC1 |
| nrg1, cplx1, tph1, gsk3a, gsk3b, nrxn1, | NRG1, CPLX1, TPH1, GSK3A, GSK3B, |
| bdnf | NRXN1, BDNF |
| nrg1, cplx1, tph1, gsk3a, gsk3b, nrxn1, | NRG1, CPLX1, TPH1, GSK3A, GSK3B, |
| disc1 | NRXN1, DISC1 |
| nrg1, cplx1, tph1, gsk3a, gsk3b, bdnf, | NRG1, CPLX1, TPH1, GSK3A, GSK3B, |
| disc1 | BDNF, DISC1 |
| nrg1, cplx1, tph1, gsk3a, nrxn1, bdnf, | NRG1, CPLX1, TPH1, GSK3A, NRXN1, |
| disc1 | BDNF, DISC1 |
| nrg1, cplx1, tph1, gsk3b, nrxn1, bdnf, | NRG1, CPLX1, TPH1, GSK3B, NRXN1, |
| disc1 | BDNF, DISC1 |
| nrg1, cplx1, tph2, gsk3a, gsk3b, nrxn1, | NRG1, CPLX1, TPH2, GSK3A, GSK3B, |
| bdnf | NRXN1, BDNF |
| nrg1, cplx1, tph2, gsk3a, gsk3b, nrxn1, | NRG1, CPLX1, TPH2, GSK3A, GSK3B, |
| disc1 | NRXN1, DISC1 |
| nrg1, cplx1, tph2, gsk3a, gsk3b, bdnf, | NRG1, CPLX1, TPH2, GSK3A, GSK3B, |
| disc1 | BDNF, DISC1 |
| nrg1, cplx1, tph2, gsk3a, nrxn1, bdnf, | NRG1, CPLX1, TPH2, GSK3A, NRXN1, |
| disc1 | BDNF, DISC1 |
| nrg1, cplx1, tph2, gsk3b, nrxn1, bdnf, | NRG1, CPLX1, TPH2, GSK3B, NRXN1, |
| disc1 | BDNF, DISC1 |
| nrg1, cplx1, gsk3a, gsk3b, nrxn1, bdnf, | NRG1, CPLX1, GSK3A, GSK3B, NRXN1, |
| disc1 | BDNF, DISC1 |
| nrg1, tph1, tph2, gsk3a, gsk3b, nrxn1, | NRG1, TPH1, TPH2, GSK3A, GSK3B, |
| bdnf | NRXN1, BDNF |
| nrg1, tph1, tph2, gsk3a, gsk3b, nrxn1, | NRG1, TPH1, TPH2, GSK3A, GSK3B, |
| disc1 | NRXN1, DISC1 |
| nrg1, tph1, tph2, gsk3a, gsk3b, bdnf, disc1 | NRG1, TPH1, TPH2, GSK3A, GSK3B, |
| | BDNF, DISC1 |
| nrg1, tph1, tph2, gsk3a, nrxn1, bdnf, disc1 | NRG1, TPH1, TPH2, GSK3A, NRXN1, |
| | BDNF, DISC1 |
| nrg1, tph1, tph2, gsk3b, nrxn1, bdnf, disc1 | NRG1, TPH1, TPH2, GSK3B, NRXN1, |
| | BDNF, DISC1 |
| nrg1, tph1, gsk3a, gsk3b, nrxn1, bdnf, | NRG1, TPH1, GSK3A, GSK3B, NRXN1, |
| disc1 | BDNF, DISC1 |
| nrg1, tph2, gsk3a, gsk3b, nrxn1, bdnf, | NRG1, TPH2, GSK3A, GSK3B, NRXN1, |
| disc1 | BDNF, DISC1 |
| erbb4, cplx1, tph1, tph2, gsk3a, gsk3b, | ErbB4, CPLX1, TPH1, TPH2, GSK3A, |
| nrxn1 | GSK3B, NRXN1 |
| erbb4, cplx1, tph1, tph2, gsk3a, gsk3b, | ErbB4, CPLX1, TPH1, TPH2, GSK3A, |
| bdnf | GSK3B, BDNF |
| erbb4, cplx1, tph1, tph2, gsk3a, gsk3b, | ErbB4, CPLX1, TPH1, TPH2, GSK3A, |
| disc1 | GSK3B, DISC1 |
| erbb4, cplx1, tph1, tph2, gsk3a, nrxn1, | ErbB4, CPLX1, TPH1, TPH2, GSK3A, |
| bdnf | NRXN1, BDNF |
| erbb4, cplx1, tph1, tph2, gsk3a, nrxn1, | ErbB4, CPLX1, TPH1, TPH2, GSK3A, |
| disc1 | NRXN1, DISC1 |
| erbb4, cplx1, tph1, tph2, gsk3a, bdnf, | ErbB4, CPLX1, TPH1, TPH2, GSK3A, |
| disc1 | BDNF, DISC1 |
| erbb4, cplx1, tph1, tph2, gsk3b, nrxn1, | ErbB4, CPLX1, TPH1, TPH2, GSK3B, |
| bdnf | NRXN1, BDNF |
| erbb4, cplx1, tph1, tph2, gsk3b, nrxn1, | ErbB4, CPLX1, TPH1, TPH2, GSK3B, |
| disc1 | NRXN1, DISC1 |
| erbb4, cplx1, tph1, tph2, gsk3b, bdnf, | ErbB4, CPLX1, TPH1, TPH2, GSK3B, |
| disc1 | BDNF, DISC1 |
| erbb4, cplx1, tph1, tph2, nrxn1, bdnf, | ErbB4, CPLX1, TPH1, TPH2, NRXN1, |
| disc1 | BDNF, DISC1 |
| erbb4, cplx1, tph1, gsk3a, gsk3b, nrxn1, | ErbB4, CPLX1, TPH1, GSK3A, GSK3B, |
| bdnf | NRXN1, BDNF |
| erbb4, cplx1, tph1, gsk3a, gsk3b, nrxn1, | ErbB4, CPLX1, TPH1, GSK3A, GSK3B, |
| disc1 | NRXN1, DISC1 |
| erbb4, cplx1, tph1, gsk3a, gsk3b, bdnf, | ErbB4, CPLX1, TPH1, GSK3A, GSK3B, |
| disc1 | BDNF, DISC1 |
| erbb4, cplx1, tph1, gsk3a, nrxn1, bdnf, | ErbB4, CPLX1, TPH1, GSK3A, NRXN1, |
| disc1 | BDNF, DISC1 |
| erbb4, cplx1, tph1, gsk3b, nrxn1, bdnf, | ErbB4, CPLX1, TPH1, GSK3B, NRXN1, |
| disc1 | BDNF, DISC1 |
| erbb4, cplx1, tph2, gsk3a, gsk3b, nrxn1, | ErbB4, CPLX1, TPH2, GSK3A, GSK3B, |
| bdnf | NRXN1, BDNF |
| erbb4, cplx1, tph2, gsk3a, gsk3b, nrxn1, | ErbB4, CPLX1, TPH2, GSK3A, GSK3B, |
| disc1 | NRXN1, DISC1 |
| erbb4, cplx1, tph2, gsk3a, gsk3b, bdnf, | ErbB4, CPLX1, TPH2, GSK3A, GSK3B, |
| disc1 | BDNF, DISC1 |
| erbb4, cplx1, tph2, gsk3a, nrxn1, bdnf, | ErbB4, CPLX1, TPH2, GSK3A, NRXN1, |
| disc1 | BDNF, DISC1 |
| erbb4, cplx1, tph2, gsk3b, nrxn1, bdnf, | ErbB4, CPLX1, TPH2, GSK3B, NRXN1, |
| disc1 | BDNF, DISC1 |
| erbb4, cplx1, gsk3a, gsk3b, nrxn1, bdnf, | ErbB4, CPLX1, GSK3A, GSK3B, NRXN1, |
| disc1 | BDNF, DISC1 |
| erbb4, tph1, tph2, gsk3a, gsk3b, nrxn1, | ErbB4, TPH1, TPH2, GSK3A, GSK3B, |
| bdnf | NRXN1, BDNF |
| erbb4, tph1, tph2, gsk3a, gsk3b, nrxn1, | ErbB4, TPH1, TPH2, GSK3A, GSK3B, |
| disc1 | NRXN1, DISC1 |
| erbb4, tph1, tph2, gsk3a, gsk3b, bdnf, | ErbB4, TPH1, TPH2, GSK3A, GSK3B, |
| disc1 | BDNF, DISC1 |
| erbb4, tph1, tph2, gsk3a, nrxn1, bdnf, | ErbB4, TPH1, TPH2, GSK3A, NRXN1, |
| disc1 | BDNF, DISC1 |
| erbb4, tph1, tph2, gsk3b, nrxn1, bdnf, | ErbB4, TPH1, TPH2, GSK3B, NRXN1, |
| disc1 | BDNF, DISC1 |
| erbb4, tph1, gsk3a, gsk3b, nrxn1, bdnf, | ErbB4, TPH1, GSK3A, GSK3B, NRXN1, |
| disc1 | BDNF, DISC1 |
| erbb4, tph2, gsk3a, gsk3b, nrxn1, bdnf, | ErbB4, TPH2, GSK3A, GSK3B, NRXN1, |
| disc1 | BDNF, DISC1 |
| cplx1, tph1, tph2, gsk3a, gsk3b, nrxn1, | CPLX1, TPH1, TPH2, GSK3A, GSK3B, |
| bdnf | NRXN1, BDNF |
| cplx1, tph1, tph2, gsk3a, gsk3b, nrxn1, | CPLX1, TPH1, TPH2, GSK3A, GSK3B, |
| disc1 | NRXN1, DISC1 |
| cplx1, tph1, tph2, gsk3a, gsk3b, bdnf, | CPLX1, TPH1, TPH2, GSK3A, GSK3B, |
| disc1 | BDNF, DISC1 |
| cplx1, tph1, tph2, gsk3a, nrxn1, bdnf, | CPLX1, TPH1, TPH2, GSK3A, NRXN1, |
| disc1 | BDNF, DISC1 |
| cplx1, tph1, tph2, gsk3b, nrxn1, bdnf, | CPLX1, TPH1, TPH2, GSK3B, NRXN1, |
| disc1 | BDNF, DISC1 |
| cplx1, tph1, gsk3a, gsk3b, nrxn1, bdnf, | CPLX1, TPH1, GSK3A, GSK3B, NRXN1, |
| disc1 | BDNF, DISC1 |
| cplx1, tph2, gsk3a, gsk3b, nrxn1, bdnf, | CPLX1, TPH2, GSK3A, GSK3B, NRXN1, |
| disc1 | BDNF, DISC1 |
| tph1, tph2, gsk3a, gsk3b, nrxn1, bdnf, | TPH1, TPH2, GSK3A, GSK3B, NRXN1, |
| disc1 | BDNF, DISC1 |
| nrg1, erbb4, cplx1, tph1, tph2, gsk3a, | NRG1, ErbB4, CPLX1, TPH1, TPH2, |
| gsk3b, nrxn1 | GSK3A, GSK3B, NRXN1 |
| nrg1, erbb4, cplx1, tph1, tph2, gsk3a, | NRG1, ErbB4, CPLX1, TPH1, TPH2, |
| gsk3b, bdnf | GSK3A, GSK3B, BDNF |
| nrg1, erbb4, cplx1, tph1, tph2, gsk3a, | NRG1, ErbB4, CPLX1, TPH1, TPH2, |
| gsk3b, disc1 | GSK3A, GSK3B, DISC1 |
| nrg1, erbb4, cplx1, tph1, tph2, gsk3a, | NRG1, ErbB4, CPLX1, TPH1, TPH2, |
| nrxn1, bdnf | GSK3A, NRXN1, BDNF |
| nrg1, erbb4, cplx1, tph1, tph2, gsk3a, | NRG1, ErbB4, CPLX1, TPH1, TPH2, |
| nrxn1, disc1 | GSK3A, NRXN1, DISC1 |
| nrg1, erbb4, cplx1, tph1, tph2, gsk3a, | NRG1, ErbB4, CPLX1, TPH1, TPH2, |
| bdnf, disc1 | GSK3A, BDNF, DISC1 |
| nrg1, erbb4, cplx1, tph1, tph2, gsk3b, | NRG1, ErbB4, CPLX1, TPH1, TPH2, |
| nrxn1, bdnf | GSK3B, NRXN1, BDNF |
| nrg1, erbb4, cplx1, tph1, tph2, gsk3b, | NRG1, ErbB4, CPLX1, TPH1, TPH2, |
| nrxn1, disc1 | GSK3B, NRXN1, DISC1 |
| nrg1, erbb4, cplx1, tph1, tph2, gsk3b, | NRG1, ErbB4, CPLX1, TPH1, TPH2, |
| bdnf, disc1 | GSK3B, BDNF, DISC1 |
| nrg1, erbb4, cplx1, tph1, tph2, nrxn1, bdnf, | NRG1, ErbB4, CPLX1, TPH1, TPH2, |
| disc1 | NRXN1, BDNF, DISC1 |
| nrg1, erbb4, cplx1, tph1, gsk3a, gsk3b, | NRG1, ErbB4, CPLX1, TPH1, GSK3A, |
| nrxn1, bdnf | GSK3B, NRXN1, BDNF |
| nrg1, erbb4, cplx1, tph1, gsk3a, gsk3b, | NRG1, ErbB4, CPLX1, TPH1, GSK3A, |
| nrxn1, disc1 | GSK3B, NRXN1, DISC1 |
| nrg1, erbb4, cplx1, tph1, gsk3a, gsk3b, | NRG1, ErbB4, CPLX1, TPH1, GSK3A, |
| bdnf, disc1 | GSK3B, BDNF, DISC1 |
| nrg1, erbb4, cplx1, tph1, gsk3a, nrxn1, | NRG1, ErbB4, CPLX1, TPH1, GSK3A, |
| bdnf, disc1 | NRXN1, BDNF, DISC1 |
| nrg1, erbb4, cplx1, tph1, gsk3b, nrxn1, | NRG1, ErbB4, CPLX1, TPH1, GSK3B, |
| bdnf, disc1 | NRXN1, BDNF, DISC1 |
| nrg1, erbb4, cplx1, tph2, gsk3a, gsk3b, | NRG1, ErbB4, CPLX1, TPH2, GSK3A, |
| nrxn1, bdnf | GSK3B, NRXN1, BDNF |
| nrg1, erbb4, cplx1, tph2, gsk3a, gsk3b, | NRG1, ErbB4, CPLX1, TPH2, GSK3A, |
| nrxn1, disc1 | GSK3B, NRXN1, DISC1 |
| nrg1, erbb4, cplx1, tph2, gsk3a, gsk3b, | NRG1, ErbB4, CPLX1, TPH2, GSK3A, |
| bdnf, disc1 | GSK3B, BDNF, DISC1 |
| nrg1, erbb4, cplx1, tph2, gsk3a, nrxn1, | NRG1, ErbB4, CPLX1, TPH2, GSK3A, |
| bdnf, disc1 | NRXN1, BDNF, DISC1 |
| nrg1, erbb4, cplx1, tph2, gsk3b, nrxn1, | NRG1, ErbB4, CPLX1, TPH2, GSK3B, |
| bdnf, disc1 | NRXN1, BDNF, DISC1 |
| nrg1, erbb4, cplx1, gsk3a, gsk3b, nrxn1, | NRG1, ErbB4, CPLX1, GSK3A, GSK3B, |
| bdnf, disc1 | NRXN1, BDNF, DISC1 |
| nrg1, erbb4, tph1, tph2, gsk3a, gsk3b, | NRG1, ErbB4, TPH1, TPH2, GSK3A, |
| nrxn1, bdnf | GSK3B, NRXN1, BDNF |
| nrg1, erbb4, tph1, tph2, gsk3a, gsk3b, | NRG1, ErbB4, TPH1, TPH2, GSK3A, |
| nrxn1, disc1 | GSK3B, NRXN1, DISC1 |
| nrg1, erbb4, tph1, tph2, gsk3a, gsk3b, | NRG1, ErbB4, TPH1, TPH2, GSK3A, |
| bdnf, disc1 | GSK3B, BDNF, DISC1 |
| nrg1, erbb4, tph1, tph2, gsk3a, nrxn1, | NRG1, ErbB4, TPH1, TPH2, GSK3A, |
| bdnf, disc1 | NRXN1, BDNF, DISC1 |
| nrg1, erbb4, tph1, tph2, gsk3b, nrxn1, | NRG1, ErbB4, TPH1, TPH2, GSK3B, |
| bdnf, disc1 | NRXN1, BDNF, DISC1 |
| nrg1, erbb4, tph1, gsk3a, gsk3b, nrxn1, | NRG1, ErbB4, TPH1, GSK3A, GSK3B, |
| bdnf, disc1 | NRXN1, BDNF, DISC1 |
| nrg1, erbb4, tph2, gsk3a, gsk3b, nrxn1, | NRG1, ErbB4, TPH2, GSK3A, GSK3B, |
| bdnf, disc1 | NRXN1, BDNF, DISC1 |
| nrg1, cplx1, tph1, tph2, gsk3a, gsk3b, | NRG1, CPLX1, TPH1, TPH2, GSK3A, |
| nrxn1, bdnf | GSK3B, NRXN1, BDNF |
| nrg1, cplx1, tph1, tph2, gsk3a, gsk3b, | NRG1, CPLX1, TPH1, TPH2, GSK3A, |
| nrxn1, disc1 | GSK3B, NRXN1, DISC1 |
| nrg1, cplx1, tph1, tph2, gsk3a, gsk3b, | NRG1, CPLX1, TPH1, TPH2, GSK3A, |
| bdnf, disc1 | GSK3B, BDNF, DISC1 |
| nrg1, cplx1, tph1, tph2, gsk3a, nrxn1, | NRG1, CPLX1, TPH1, TPH2, GSK3A, |
| bdnf, disc1 | NRXN1, BDNF, DISC1 |
| nrg1, cplx1, tph1, tph2, gsk3b, nrxn1, | NRG1, CPLX1, TPH1, TPH2, GSK3B, |
| bdnf, disc1 | NRXN1, BDNF, DISC1 |
| nrg1, cplx1, tph1, gsk3a, gsk3b, nrxn1, | NRG1, CPLX1, TPH1, GSK3A, GSK3B, |
| bdnf, disc1 | NRXN1, BDNF, DISC1 |
| nrg1, cplx1, tph2, gsk3a, gsk3b, nrxn1, | NRG1, CPLX1, TPH2, GSK3A, GSK3B, |
| bdnf, disc1 | NRXN1, BDNF, DISC1 |
| nrg1, tph1, tph2, gsk3a, gsk3b, nrxn1, | NRG1, TPH1, TPH2, GSK3A, GSK3B, |
| bdnf, disc1 | NRXN1, BDNF, DISC1 |
| erbb4, cplx1, tph1, tph2, gsk3a, gsk3b, | ErbB4, CPLX1, TPH1, TPH2, GSK3A, |
| nrxn1, bdnf | GSK3B, NRXN1, BDNF |
| erbb4, cplx1, tph1, tph2, gsk3a, gsk3b, | ErbB4, CPLX1, TPH1, TPH2, GSK3A, |
| nrxn1, disc1 | GSK3B, NRXN1, DISC1 |
| erbb4, cplx1, tph1, tph2, gsk3a, gsk3b, | ErbB4, CPLX1, TPH1, TPH2, GSK3A, |
| bdnf, disc1 | GSK3B, BDNF, DISC1 |
| erbb4, cplx1, tph1, tph2, gsk3a, nrxn1, | ErbB4, CPLX1, TPH1, TPH2, GSK3A, |
| bdnf, disc1 | NRXN1, BDNF, DISC1 |
| erbb4, cplx1, tph1, tph2, gsk3b, nrxn1, | ErbB4, CPLX1, TPH1, TPH2, GSK3B, |
| bdnf, disc1 | NRXN1, BDNF, DISC1 |
| erbb4, cplx1, tph1, gsk3a, gsk3b, nrxn1, | ErbB4, CPLX1, TPH1, GSK3A, GSK3B, |
| bdnf, disc1 | NRXN1, BDNF, DISC1 |
| erbb4, cplx1, tph2, gsk3a, gsk3b, nrxn1, | ErbB4, CPLX1, TPH2, GSK3A, GSK3B, |
| bdnf, disc1 | NRXN1, BDNF, DISC1 |
| erbb4, tph1, tph2, gsk3a, gsk3b, nrxn1, | ErbB4, TPH1, TPH2, GSK3A, GSK3B, |
| bdnf, disc1 | NRXN1, BDNF, DISC1 |
| cplx1, tph1, tph2, gsk3a, gsk3b, nrxn1, | CPLX1, TPH1, TPH2, GSK3A, GSK3B, |
| bdnf, disc1 | NRXN1, BDNF, DISC1 |
| nrg1, erbb4, cplx1, tph1, tph2, gsk3a, | NRG1, ErbB4, CPLX1, TPH1, TPH2, |
| gsk3b, nrxn1, bdnf | GSK3A, GSK3B, NRXN1, BDNF |
| nrg1, erbb4, cplx1, tph1, tph2, gsk3a, | NRG1, ErbB4, CPLX1, TPH1, TPH2, |
| gsk3b, nrxn1, disc1 | GSK3A, GSK3B, NRXN1, DISC1 |
| nrg1, erbb4, cplx1, tph1, tph2, gsk3a, | NRG1, ErbB4, CPLX1, TPH1, TPH2, |
| gsk3b, bdnf, disc1 | GSK3A, GSK3B, BDNF, DISC1 |
| nrg1, erbb4, cplx1, tph1, tph2, gsk3a, | NRG1, ErbB4, CPLX1, TPH1, TPH2, |
| nrxn1, bdnf, disc1 | GSK3A, NRXN1, BDNF, DISC1 |
| nrg1, erbb4, cplx1, tph1, tph2, gsk3b, | NRG1, ErbB4, CPLX1, TPH1, TPH2, |
| nrxn1, bdnf, disc1 | GSK3B, NRXN1, BDNF, DISC1 |
| nrg1, erbb4, cplx1, tph1, gsk3a, gsk3b, | NRG1, ErbB4, CPLX1, TPH1, GSK3A, |
| nrxn1, bdnf, disc1 | GSK3B, NRXN1, BDNF, DISC1 |
| nrg1, erbb4, cplx1, tph2, gsk3a, gsk3b, | NRG1, ErbB4, CPLX1, TPH2, GSK3A, |
| nrxn1, bdnf, disc1 | GSK3B, NRXN1, BDNF, DISC1 |
| nrg1, erbb4, tph1, tph2, gsk3a, gsk3b, | NRG1, ErbB4, TPH1, TPH2, GSK3A, |
| nrxn1, bdnf, disc1 | GSK3B, NRXN1, BDNF, DISC1 |
| nrg1, cplx1, tph1, tph2, gsk3a, gsk3b, | NRG1, CPLX1, TPH1, TPH2, GSK3A, |
| nrxn1, bdnf, disc1 | GSK3B, NRXN1, BDNF, DISC1 |
| erbb4, cplx1, tph1, tph2, gsk3a, gsk3b, | ErbB4, CPLX1, TPH1, TPH2, GSK3A, |
| nrxn1, bdnf, disc1 | GSK3B, NRXN1, BDNF, DISC1 |
| nrg1, erbb4, cplx1, tph1, tph2, gsk3a, | NRG1, ErbB4, CPLX1, TPH1, TPH2, |
| gsk3b, nrxn1, bdnf, disc1 | GSK3A, GSK3B, NRXN1, BDNF, DISC1 |

In one aspect, the chromosomal sequences of any combination of any two exemplary proteins associated with schizophrenia may be edited using a zinc finger nuclease-mediated process. In other aspects, the chromosomal sequences of any combination of any three, any four, any five, any six, any seven, any eight, any nine, or any ten exemplary proteins associated with schizophrenia may be edited using a zinc finger nuclease-mediated process. In yet another aspect, the chromosomal sequences of any combination of all ten exemplary proteins associated with schizophrenia may be edited using a zinc finger nuclease-mediated process.

Exemplary genetically modified animals may comprise one, two, three, four, five, six, seven, eight, nine, or ten inactivated chromosomal sequences encoding proteins associated with schizophrenia and zero, one, two, three, four, five, six, seven, eight, nine, or ten chromosomally integrated sequences encoding orthologous or modified proteins associated with schizophrenia.

### (b) Animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include, but are not limited to, capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of commonly used rat strains suitable for genetic manipulation include Dahl Salt-Sensitive, Fischer 344, Lewis, Long Evans Hooded, Sprague-Dawley and Wistar. In another iteration of the invention, the animal does not comprise a genetically modified mouse. In each of the foregoing iterations of suitable animals for the invention, the animal does not include exogenously introduced, randomly integrated transposon sequences.

(c) Proteins Associated with Schizophrenia

The protein associated with schizophrenia may be from any of the animals listed above. Furthermore, the protein associated with schizophrenia may be a human protein. Additionally, the protein associated with schizophrenia may be a bacterial, fungal, or plant protein. The type of animal and the source of the protein can and will vary. As an example, the genetically modified animal may be a rat, cat, dog, or pig, and the protein associated with schizophrenia may be human. Alternatively, the genetically modified animal may be a rat, cat, or pig, and the protein associated with schizophrenia may be canine. One of skill in the art will readily appreciate that numerous combinations are possible.

Additionally, the gene associated with schizophrenia may be modified to include a tag or reporter gene or genes as are well-known. Reporter genes include those encoding selectable markers such as cloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

### (II) Genetically Modified Cells

A further aspect of the present disclosure provides genetically modified cells or cell lines comprising at least one edited chromosomal sequence associated with schizophrenia. The genetically modified cell or cell line may be derived from any of the genetically modified animals disclosed herein. Alternatively, the chromosomal sequence associated with schizophrenia may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CV1-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Mamassas, Va.).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

In a preferred embodiment the chromosomal sequence may be targeted for editing in any of the following commonly used rat strains: Dahl Salt-Sensitive, Fischer 344, Lewis, Long Evans Hooded, Sprague-Dawley, or Wistar.

### (III) Zinc Finger-Mediated Genome Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

### (a) Zinc Finger Nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) Zinc Finger Binding Domain

Zinc finger binding domains may be engineered to recognize and bind to any nucleic acid sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; and Choo et al. (2000) Curr. Opin. Struct. Biol. 10:411-416. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (see, for example, Biochemistry 2002, 41, 7074-7081).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence that facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

An exemplary zinc finger DNA binding domain recognizes and binds a sequence having at least about 80% sequence identity with a sequence chosen from SEQ ID NOs: 4, 5, 6 and 7. In other embodiments, the sequence identity may be about 81%, 82%, 83%, 84%, 85%, 86%, 87%, 88%, 89%, 90%, 91%, 92%, 93%, 94%, 95%, 96%, 97%, 98%, 99%, or 100%.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (O) with Glu (E); and a mutation at position 499 replaces Iso (I) with Lys (K). Specifically, the engineered cleavage monomers may be prepared by mutating positions 490 from E to K and 538 from 1 to K in one cleavage monomer to produce an engineered cleavage monomer designated “E490K:I538K” and by mutating positions 486 from Q to E and 499 from Ito L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Donor Polynucleotide

The method for editing chromosomal sequences associated with schizophrenia may further comprise introducing at least one donor polynucleotide comprising a sequence encoding a protein associated with schizophrenia into the embryo or cell. A donor polynucleotide comprises at least three components: the sequence coding the protein associated with schizophrenia, an upstream sequence, and a downstream sequence. The sequence encoding the protein associated with schizophrenia is flanked by the upstream and downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding a protein associated with schizophrenia may be a BAC.

The sequence of the donor polynucleotide that encodes the protein associated with schizophrenia may include coding (i.e., exon) sequence, as well as intron sequences and upstream regulatory sequences (such as, e.g., a promoter). Depending upon the identity and the source of the protein associated with schizophrenia, the size of the sequence encoding the protein associated with schizophrenia can and will vary. For example, the sequence encoding the protein associated with schizophrenia may range in size from about 1 kb to about 5,000 kb.

The donor polynucleotide also comprises upstream and downstream sequences flanking the chromosomal sequence associated with schizophrenia. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 bp to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a chromosomal sequence associated with schizophrenia, a double stranded break introduced into the chromosomal sequence by the zinc finger nuclease is repaired, via homologous recombination with the donor polynucleotide, such that the chromosomal sequence associated with schizophrenia is integrated into the chromosome. The presence of a double-stranded break facilitates integration of the sequence into the chromosome. A donor polynucleotide may be physically integrated or, alternatively, the donor polynucleotide may be used as a template for repair of the break, resulting in the introduction of the chromosomal sequence associated with schizophrenia as well as all or part of the upstream and downstream sequences of the donor polynucleotide into the chromosome. Thus, endogenous chromosomal sequence may be converted to the sequence of the donor polynucleotide.

### (c) Optional Exchange Polynucleotide

The method for editing chromosomal sequences associated with schizophrenia may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85, 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 bp to about 10,000 bp in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (d) Delivery of Nucleic Acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo of the species of interest.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides, may be introduced at the same time. Alternatively, each nucleic acid encoding a zinc finger nuclease, as well as the optional donor (or exchange) polynucleotides, may be introduced sequentially

### (e) Culturing the Embryo or Cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking, the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence associated with schizophrenia in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion, or nonsense mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified).

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated NRG1 chromosomal sequence may be crossed with animal B comprising a chromosomally integrated sequence encoding a human NRG1 protein to give rise to a “humanized” NRG 1 offspring comprising both the inactivated NRG1 chromosomal sequence and the chromosomally integrated human NRG1 sequence. Similarly, an animal comprising an inactivated ErbB4 chromosomal sequence may be crossed with an animal comprising a chromosomally integrated sequence encoding the human ErbB4 protein to generate “humanized” ErbB4 offspring. Moreover, a humanized NRG1 animal may be crossed with a humanized ErbB4 animal to create a humanized NRG1/ErbB4 animal. Those of skill in the art will appreciate that many combinations are possible.

In other embodiments, an animal comprising an edited chromosomal sequence disclosed herein may be crossbred to combine the edited chromosomal sequence with other genetic backgrounds. By way of non-limiting example, other genetic backgrounds may include wild-type genetic backgrounds, genetic backgrounds with deletion mutations, genetic backgrounds with another targeted integration, and genetic backgrounds with non-targeted integrations. Suitable integrations may include, without limit, nucleic acids encoding drug transporter proteins, Mdr protein, and the like.

### (IV) Applications

A further aspect of the present disclosure encompasses a method for assessing an effect of an agent such as a pharmaceutically active ingredient, a drug, a toxin, or a chemical. For example, the effect of an agent may be measured in a “humanized” genetically modified animal, such that the information gained therefrom may be used to predict the effect of the agent in a human. In general, the method comprises administering the agent to a genetically modified animal comprising at least one inactivated chromosomal sequence encoding a protein associated with schizophrenia and at least one chromosomally integrated sequence encoding an orthologous protein associated with schizophrenia, and comparing a parameter obtained from the genetically modified animal to the parameter obtained from a wild-type animal administered the same agent. Suitable agents include without limit pharmaceutically active ingredients, drugs, foods, food additives, pesticides, herbicides, toxins, industrial chemicals, household chemicals, and other environmental chemicals. The agent may be a therapeutic treatment for a schizophrenia-related disorder, including but not limited to administering of one or more novel candidate therapeutic compounds, administering a novel combination of established therapeutic compounds, a novel therapeutic method, and any combination thereof. Non-limiting examples of novel therapeutic methods include various drug delivery mechanisms such as oral or injected therapeutic compositions, drug-releasing implants, nanotechnology applications in drug therapy, vaccine compositions, surgery, and combinations thereof.

Non-limiting examples of suitable parameters for the assessment of the agent include: (a) rate of elimination of the agent or at least one agent metabolite; (b) circulatory levels of the agent or at least one agent metabolite; (c) bioavailability of the agent or at least one agent metabolite; (d) rate of metabolism of the agent or at least one agent metabolite; (e) rate of clearance of the agent or at least one agent metabolite; (f) toxicity of the agent or at least one agent metabolite; (g) efficacy of the agent or at least one agent metabolite; (h) disposition of the agent or at least one agent metabolite; and (i) extrahepatic contribution to metabolic rate and clearance of the agent or at least one agent metabolite; and (j) ability of the agent to modify an incidence or indication of schizophrenia or a related disorder in the genetically modified animal.

For example, an ADME-Tox profile of an agent may be assessed using the genetically modified animal. The ADME-Tox profile may include assessments of at least one or more physiologic and metabolic consequences of administering the agent. In addition, the ADME-Tox profile may assess behavioral effects such as addiction or depression in response to the agent.

The incidence or indication of the schizophrenia or related disorder may occur spontaneously in the genetically modified animal. Alternatively, the incidence or indication of the schizophrenia or related disorder may be promoted by exposure to a disruptive agent. Non-limiting examples of disruptive agents include a protein associated with schizophrenia such as any of those described above, a drug, a toxin, a chemical, an activated retrovirus, and an environmental stress. Non-limiting examples of environmental stresses include forced swimming, cold swimming, platform shaker stimuli, loud noises, and immobilization stress.

Suitable proteins associated with schizophrenia may include any one or more of proteins associated with schizophrenia described above, including but not limited to NRG1, ErbB4, CPLX1, TPH1, TPH2, NRXN1, GSK3A, BDNF, DISC1, GSK3B, and combinations thereof.

Yet another aspect encompasses a method for assessing the therapeutic potential of an agent as a treatment for schizophrenia or a related disorder. The method includes administering the agent to a genetically modified animal and comparing a selected parameter obtained from the genetically modified animal to the selected parameter obtained from a wild-type animal with no exposure to the same agent. The genetically modified animal comprises at least one edited chromosomal sequence encoding a protein associated with schizophrenia. The selected parameter may be chosen from a) spontaneous behaviors; b) performance during behavioral testing; c) physiological anomalies; d) abnormalities in tissues or cells; e) biochemical function; and f) molecular structures. These selected parameters may also be used to assess a genetically modified animal for one or more indications of a schizophrenia-related disorder. As described previously, the genetically modified animal may develop the schizophrenia or related disorder spontaneously, or the development of the disorder may be promoted by a disruptive agent.

Spontaneous behavior may be assessed using any one or more methods of spontaneous behavioral observation known in the art. In general, any spontaneous behavior within a known behavioral repertoire of an animal may be observed, including movement, posture, social interaction, rearing, sleeping, blinking, eating, drinking, urinating, defecating, mating, and aggression. An extensive battery of observations for quantifying the spontaneous behavior of mice and rats is well-known in the art, including but not limited to home-cage observations such as body position, respiration, tonic involuntary movement, unusual motor behavior such as pacing or rocking, catatonic behavior, vocalization, palpebral closure, mating frequency, running wheel behavior, nest building, and frequency of aggressive interactions.

Performance during behavioral testing may be assessed using any number of behavioral tests known in the art. The particular type of performance test may depend upon at least one of several factors including the behavioral repertoire of the animal and the purpose of the testing. Non-limiting examples of tests for assessing the reflex function of rats include assessments of approach response, touch response, eyelid reflex, pinna reflex, sound response, tail pinch response, pupillary reflex, and righting reflex. Non-limiting examples of behavioral tests suitable for assessing the motor function of rats includes open field locomotor activity assessment, the rotarod test, the grip strength test, the cylinder test, the limb-placement or grid walk test, the vertical pole test, the Inverted grid test, the adhesive removal test, the painted paw or catwalk (gait) tests, the beam traversal test, and the inclined plane test. Non-limiting examples of behavioral tests suitable for assessing the long-term memory function of rats include the elevated plus maze test, the Morris water maze swim test, contextual fear conditioning, the Y-maze test, the T-maze test, the novel object recognition test, the active avoidance test, the passive (inhibitory) avoidance test, the radial arm maze test, the two-choice swim test, the hole board test, the olfactory discrimination (go-no-go) test, and the pre-pulse inhibition test. Non-limiting examples of behavioral tests suitable for assessing the anxiety of rats include the open field locomotion assessment, observations of marble-burying behavior, the elevated plus maze test, the light/dark box test. Non-limiting examples of behavioral tests suitable for assessing the depression of rats includes the forced swim test, the tail suspension test, the hot plate test, the tail suspension test, anhedonia observations, and the novelty suppressed feeding test.

Physiological anomalies may include any difference in physiological function between a genetically modified animal and a wild-type animal. Non-limiting examples of physiological functions include homeostasis, metabolism, sensory function, neurological function, musculoskeletal function, cardiovascular function, respiratory function, dermatological function, renal function, reproductive functions, immunological function, and endocrinological function. Numerous measures of physiological function are well-known in the art.

Abnormalities in tissues or cells may include any difference in the structure or function of a tissue or cell of a genetically modified animal and the corresponding structure or function of a wild-type animal. Non-limiting examples of cell or tissue abnormalities include cell hypertrophy, tissue hyperplasia, neoplasia, hypoplasia, aplasia, hypotrophy, dysplasia, overproduction or underproduction of cell products, abnormal neuronal discharge frequency, and changes in synaptic density of neurons.

Non-limiting examples of biochemical functions may include enzyme function, cell signaling function, maintenance of homeostasis, cellular respiration; methods of assessing biochemical functions are well known in the art. Molecular structures may be assessed using any method known in the art including microscopy such as dual-photon microscopy and scanning electron microscopy, and immunohistological techniques such as Western blot and ELISA.

A additional aspect provides a method for assessing a side effect of a therapeutic compound comprising administering the therapeutic compound to an animal model and assessing at least one or more behaviors chosen from learning, memory, anxiety, depression, addiction, sensory-motor function, taste preference, and odor preference. The animal model may be chosen from a genetically modified animal and a wild-type animal. The genetically modified animal comprises at least one edited chromosomal sequence encoding a protein associated with schizophrenia. The therapeutic compound is chosen from a novel therapeutic compound and a novel combination of known therapeutic agents. Any of the methods described above to measure spontaneous behavior or performance during behavioral tests may be used to assess the side effect.

In this method, the therapeutic compound may be self-administered, or the therapeutic compound may be administered by another. The animal model may be contacted with the therapeutic compound using administration methods including oral ingestion, epidermal absorption, injection, absorption through the mucous membranes of the oral cavity, rectum, nasal cavity, lungs, or vagina, and any other suitable administration method known in the art. If the therapeutic compound is administered using oral ingestion, the therapeutic compound may be incorporated in an amount of water, food, or supplemental material such as a chewable or lickable object and provided to the animal model.

Also provided are methods to assess an effect of an agent in an isolated cell comprising at least one edited chromosomal sequence encoding a protein associated with schizophrenia, as well as methods of using lysates of such cells (or cells derived from a genetically modified animal disclosed herein) to assess the effect of an agent. For example, the role of a particular protein associated with schizophrenia in the metabolism of a particular agent may be determined using such methods. Similarly, substrate specificity and pharmacokinetic parameter may be readily determined using such methods. Those of skill in the art are familiar with suitable tests and/or procedures.

Yet another aspect encompasses a method for assessing the therapeutic efficacy of a potential gene therapy strategy. That is, a chromosomal sequence encoding a protein associated with schizophrenia may be modified such that the incidence or indications of schizophrenia or a related disorder of a genetically modified animal are reduced or eliminated. In particular, the method comprises editing a chromosomal sequence encoding a protein associated with schizophrenia such that an altered protein product is produced. The genetically modified animal may be exposed to a disruptive agent described above and behavioral, cellular, and/or molecular responses may be measured and compared to those of a wild-type animal exposed to the same disruptive agent. Consequently, the therapeutic potential of the schizophrenia-related gene therapy regime may be assessed.

Still yet another aspect encompasses a method of generating a cell line or cell lysate using a genetically modified animal comprising an edited chromosomal sequence encoding a protein associated with schizophrenia. An additional other aspect encompasses a method of producing purified biological components using a genetically modified cell or animal comprising an edited chromosomal sequence encoding a protein associated with schizophrenia. Non-limiting examples of biological components include antibodies, cytokines, signal proteins, enzymes, receptor agonists and receptor antagonists.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “chromosomal sequence associated with schizophrenia” refers to a chromosomal sequence that has been identified to contribute to the development of, diagnosis for, symptoms of, or treatment for schizophrenia. The term “chromosomal sequence associated with schizophrenia” may also refer to a chromosomal sequence, which has been experimentally associated with the development of, diagnosis for, or symptoms of schizophrenia. Exemplary chromosomal sequences associated with schizophrenia include, but are not limited to NRG1, ErbB4, CPLX1, TPH1, TPH2, NRXN1, GSK3A, BDNF, DISC1, GSK3B, and combinations thereof. Any chromosomal sequence known to be associated with schizophrenia will work for purposes of the present invention.

The term “a protein encoded by a chromosomal sequence associated with schizophrenia” or “a protein associated with schizophrenia” refers to a protein that has been encoded by a chromosomal sequence identified to contribute to the development of, diagnosis for, symptoms of, or treatment for schizophrenia. Exemplary proteins associated with schizophrenia include, but are not limited to, Neuroregulin1, ErbB4, Complexin 1, Tryptophan hydroxylase 1, Tryptophan hydroxylase 2, Neurexin 1-alpha, Glycogen synthase kinase-3 alpha, and Glycogen synthase kinase-3 beta. Any type of protein associated with schizophrenia will work for purposes of the present invention including, but not limited to, structural proteins, enzyme and catalytic proteins, transport proteins, hormonal proteins, contractile proteins, storage proteins, genetic proteins, defense proteins, and receptor proteins.

A “gene,” as used herein, refers to a DNA region (including exons and introns) encoding a gene product, as well as all DNA regions, which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations-FSwiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Identification of ZFNs that Edit the DISC1 Locus

The DISC1 gene in rat was chosen for zinc finger nuclease (ZFN) mediated genome editing. ZFNs were designed, assembled, and validated using strategies and procedures previously described (see Geurts et al. Science (2009) 325:433). ZFN design made use of an archive of pre-validated 1-finger and 2-finger modules. The DISC1 gene region (NM_{—}175596) was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding each pair of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay revealed that the ZFN pair targeted to bind 5′-taGTCCCGGCAGGCTATcctgggcggtg-3′ (SEQ ID NO: 4; contact sites in uppercase) and 5′-ccGTCACCAGGCGGGACtggctgatgcg-3′ (SEQ ID NO: 5) cleaved within the DISC1 locus.

### Example 2

### Editing the DISC1 Locus in Rat Embryos

Capped, polyadenylated mRNA encoding the active pair of ZFNs was microinjected into fertilized rat embryos using standard procedures (e.g., see Geurts et al. (2009) supra). The injected embryos were either incubated in vitro, or transferred to pseudopregnant female rats to be carried to parturition. The resulting embryos/fetus, or the toe/tail clip of live born animals were harvested for DNA extraction and analysis. DNA was isolated using standard procedures. The targeted region of the DISC1 locus was PCR amplified using appropriate primers. The amplified DNA was subcloned into a suitable vector and sequenced using standard methods. FIG. 1 presents an edited DISC1 locus in which 20 bp was deleted from the target sequence in exon 5. This deletion disrupts the reading frame of the DISC1 coding region.

### Example 3

### Identification of ZFNs that Edit the BDNF Locus

To identify ZFNs that target and cleave the BDNF locus, the rat BDNF gene (NM_{—}012513) was scanned for putative zinc finger binding sites. The ZFNs were assembled and tested essentially as described in Example 1. This analysis revealed that the ZFN pair targeted to bind 5′-cgGGGTCGGAGtGGCGCCgaaccctcat-3′ (SEQ ID NO: 6) and 5′-ccGCCGTGGGGaGCTGAGcgtgtgtgac-3′ (SEQ ID NO: 7) cleaved within the BDNF locus.

Fertilized rat embryos were microinjected with mRNAs encoding the active ZNF pair and analyzed essentially as described above in Example 2. FIG. 2 presents edited BDNF loci in two founder animals; one had a 14 bp deletion in the target sequence in exon 2 and the other had a 7 bp deletion in the target sequence in exon 2.

The genetically modified rats were observed for phenotypic changes. Homozygous animals died within 2 weeks of birth. Heterozygous and homozygous animals were smaller in size than corresponding control animals (i.e., derived from embryos microinjected with GFP mRNA).

### Example 4

### Genome Editing of ErbB4 in a Model Organism

ZFN-mediated genome editing may be used to study the effects of a “knockout” mutation in a chromosomal sequence associated with schizophrenia, such as a chromosomal sequence encoding the ErbB4 protein, in a genetically modified model animal and cells derived from the animal. Such a model animal may be a rat. In general, ZFNs that bind to the rat chromosomal sequence encoding the ErbB4 protein associated with schizophrenia may be used to introduce a deletion or insertion such that the coding region of the ErbB4 gene is disrupted such that a functional ErbB4 protein may not be produced.

Suitable fertilized embryos may be microinjected with capped, polyadenylated mRNA encoding the ZFN. The frequency of ZFN-induced double strand chromosomal breaks may be determined using the Cel-1 nuclease assay, as detailed above. The sequence of the edited chromosomal sequence may be analyzed as described above. The development of schizophrenia symptoms and disorders caused by the ErbB4 “knockout” may be assessed in the genetically modified rat or progeny thereof. Furthermore, molecular analyses of schizophrenia-related pathways may be performed in cells derived from the genetically modified animal comprising an ErbB4 “knockout”.

### Example 5

### Generation of a Humanized Rat Expressing a Mutant Form of Human TPH1

To develop a “humanized” animal model for the evaluation of schizophrenia symptoms and treatments, a rat comprising a genome including the human mutant form of TPH1 may be created. The human mutant form may be A218C that is found within intron 7 of TPH1; A218C is thought to be highly associated with schizophrenia. ZFN-mediated genome editing may be used to generate a humanized rat wherein the rat gene is replaced with the A218C mutant form of human TPH1. Such a humanized rat may be used to study the development of schizophrenia associated with the mutant human protein encoded by the mutated TPH1. In addition, the humanized rat may be used to assess the efficacy of potential therapeutic agents targeted at the pathway associated with TPH1.

The genetically modified rat may be generated using the methods described in Example 2 above. However, to generate the humanized rat, the ZFN mRNA may be co-injected with the human chromosomal sequence encoding the mutant TPH1 protein into the rat embryo. The rat chromosomal sequence may then be replaced by the mutant human sequence by homologous recombination, and a humanized rat expressing a mutant form of the protein may be produced.

The table below presents the amino acid sequences of helices of the active ZFNs.

| Name | Sequence of Zinc Finger Helices | SEQ ID NO: |
|---|---|---|
| DISC1 | NSGNLDK DRSHLSR QSGDLTR RSDTLSQ | 8 |
| | DRSARTR | |
| DISC1 | DRSNLSR RSDNLRE RSDHLSA DSSTRKT | 9 |
| | DRSSRKR | |
| BDNF | DRSDLSR DRSHLAR RSHNLAR RSDDLSK | 10 |
| | RSAHLSR | |
| BDNF | RSDNLAR QSSDLRR RSSHLSR RSDALSR | 11 |
| | DRSDLSR | |

What is claimed is:

1. A genetically modified animal comprising at least one edited chromosomal sequence encoding a protein associated with schizophrenia.

2. The genetically modified animal of claim 1, wherein the edited chromosomal sequence is inactivated, modified, or comprises an integrated sequence.

3. The genetically modified animal of claim 1, wherein the edited chromosomal sequence is inactivated such that no functional protein associated with schizophrenia is produced.

4. The genetically modified animal of claim 3, wherein the inactivated chromosomal sequence comprises no exogenously introduced sequence.

5. The genetically modified animal of claim 3, further comprising at least one chromosomally integrated sequence encoding a functional protein associated with schizophrenia.

6. The genetically modified animal of claim 1, wherein the protein associated with schizophrenia is chosen from NRG1, ErbB4, CPLX1, TPH1, TPH2, NRXN1, GSK3A, BDNF, DISC1, GSK3B, and combinations thereof.

7. The genetically modified animal of claim 1, further comprising a conditional knock-out system for conditional expression of the protein associated with schizophrenia.

8. The genetically modified animal of claim 1, wherein the edited chromosomal sequence comprises an integrated reporter sequence.

9. The genetically modified animal of claim 1, wherein the animal is heterozygous or homozygous for the at least one edited chromosomal sequence.

10. The genetically modified animal of claim 1, wherein the animal is an embryo, a juvenile, or an adult.

11. The genetically modified animal of claim 1, wherein the animal is chosen from bovine, canine, equine, feline, ovine, porcine, non-human primate, and rodent.

12. The genetically modified animal of claim 1, wherein the animal is rat.

13. The genetically modified animal of claim 4, wherein the animal is rat and the protein associated with schizophrenia is an ortholog of a human protein associated with schizophrenia.

14. A non-human embryo, the embryo comprising at least one RNA molecule encoding a zinc finger nuclease that recognizes a chromosomal sequence encoding a protein associated with schizophrenia, and, optionally, at least one donor polynucleotide comprising a sequence encoding an ortholog of the protein associated with schizophrenia or an edited protein associated with schizophrenia.

15. The non-human embryo of claim 14, wherein the protein associated with schizophrenia is chosen from NRG1, ErbB4, CPLX1, TPH1, TPH2, NRXN1, GSK3A, BDNF, DISC1, GSK3B, and combinations thereof.

16. The non-human embryo of claim 14, wherein the embryo is chosen from bovine, canine, equine, feline, ovine, porcine, non-human primate, and rodent.

17. The non-human embryo of claim 14, wherein the embryo is rat and the protein is an ortholog of a human protein associated with schizophrenia.

18. A genetically modified cell, the cell comprising at least one edited chromosomal sequence encoding a protein associated with schizophrenia.

19. The genetically modified cell of claim 18, wherein the edited chromosomal sequence is inactivated, modified, or comprises an integrated sequence.

20. The genetically modified cell of claim 19, wherein the edited chromosomal sequence is inactivated such that the protein associated with schizophrenia is not produced or is not functional.

21. The genetically modified cell of claim 20, further comprising at least one chromosomally integrated sequence encoding a functional protein associated with schizophrenia.

22. The genetically modified cell of claim 18, wherein the protein associated with schizophrenia is chosen from NRG1, ErbB4, CPLX1, TPH1, TPH2, NRXN1, GSK3A, BDNF, DISC1, GSK3B, and combinations thereof.

23. The genetically modified cell of claim 18, wherein the cell is heterozygous or homozygous for the at least one edited chromosomal sequence.

24. The genetically modified cell of claim 18, wherein the cell is of bovine, canine, equine, feline, human, ovine, porcine, non-human primate, or rodent origin.

25. The genetically modified cell of claim 18, wherein the cell is of rat origin and the protein is an ortholog of a human protein associated with schizophrenia.

26. The genetically modified cell of claim 20, wherein the inactivated chromosomal sequence comprises no exogenously introduced sequence.

27. The genetically modified cell of claim 18, further comprising a conditional knock-out system for conditional expression of the protein associated with schizophrenia.

28. The genetically modified cell of claim 18, wherein the edited chromosomal sequence comprises an integrated reporter sequence.

29. A zinc finger nuclease, the zinc finger nuclease comprising:
a) a zinc finger DNA binding domain that binds a sequence having at least about 80% sequence identity to a sequence chosen from SEQ ID NOs: 4, 5, 6, and 7; and
b) a cleavage domain.

30. The zinc finger nuclease of claim 29, wherein the sequence identity is at least about 85%, 90%, 95%, or 100%.

31. The zinc finger nuclease of claim 29, wherein the DNA binding domain comprises at least three zinc finger recognition regions.

32. The zinc finger nuclease of claim 29, wherein the cleavage domain is a wild-type or an engineered FokI cleavage domain.

33. A nucleic acid sequence recognized by a zinc finger nuclease, the nucleic acid sequence having at least about 80% sequence identity with a sequence chosen from SEQ ID NOs: 4, 5, 6 and 7.

34. A method for assessing the effect of an agent in a genetically modified animal, the method comprising administering the agent to the genetically modified animal comprising at least one edited chromosomal sequence encoding a protein associated with schizophrenia, and comparing a parameter obtained from the genetically modified animal to the parameter obtained from a wild-type animal administered the same agent, wherein the parameter is chosen from:
a) rate of elimination of the agent or its metabolite(s);
b) circulatory levels of the agent or its metabolite(s);
c) bioavailability of the agent or its metabolite(s);
d) rate of metabolism of the agent or its metabolite(s);
e) rate of clearance of the agent or its metabolite(s);
f) toxicity of the agent or its metabolite(s); and
g) ability of the agent to modify an incidence or indication of a schizophrenia-related disorder in the genetically modified animal.

35. The method of claim 34, wherein the agent is a pharmaceutically active ingredient, a drug, a toxin, or a chemical.

36. The method of claim 34, wherein the at least one edited chromosomal sequence is inactivated such that the protein associated with schizophrenia is not produced or is not functional, and wherein the genetically modified animal further comprises at least one chromosomally integrated sequence encoding a functional ortholog of the protein associated with schizophrenia.

37. The method of claim 34, wherein the protein associated with schizophrenia is chosen from NRG1, ErbB4, CPLX1, TPH1, TPH2, NRXN1, GSK3A, BDNF, DISC1, GSK3B, and combinations thereof.

38. The method of claim 34, wherein the animal is a rat of a strain chosen from Dahl Salt-Sensitive, Fischer 344, Lewis, Long Evans Hooded, Sprague-Dawley, and Wistar.

39. The method of claim 34, wherein the incidence or indication of the schizophrenia-related disorder occurs spontaneously in the genetically modified animal.

40. The method of claim 34, wherein the incidence or indication of the schizophrenia-related disorder is promoted by exposure to a disruptive agent.

41. The method of claim 40, wherein the disruptive agent is chosen from a protein associated with schizophrenia, a drug, a toxin, a chemical, an activated retrovirus, and an environmental stress.

42. A method for assessing the therapeutic potential of an agent as a treatment for a schizophrenia-related disorder, the method comprising administering the agent to a genetically modified animal, wherein the genetically modified animal comprises at least one edited chromosomal sequence encoding a protein associated with schizophrenia, and comparing a selected parameter obtained from the genetically modified animal to the selected parameter obtained from a wild-type animal with no exposure to the same agent, wherein the selected parameter is chosen from:
a) spontaneous behaviors;
b) performance during behavioral testing;
c) physiological anomalies;
d) abnormalities in tissues or cells;
e) biochemical function; and
f) molecular structures.

43. The method of claim 42, wherein the agent comprises at least one pharmaceutically active compound.

44. The method of claim 42, wherein the at least one edited chromosomal sequence is inactivated such that the protein associated with schizophrenia is not produced or is not functional, and wherein the animal further comprises at least one chromosomally integrated sequence encoding a functional ortholog of the protein associated with schizophrenia.

45. The method of claim 42, wherein the protein associated with schizophrenia is chosen from NRG1, ErbB4, CPLX1, TPH1, TPH2, NRXN1, GSK3A, BDNF, DISC1, GSK3B, and combinations thereof.

46. The method of claim 42, wherein the animal is a rat of a strain chosen from Dahl Salt-Sensitive, Fischer 344, Lewis, Long Evans Hooded, Sprague-Dawley, and Wistar.

47. The method of claim 42, wherein the incidence or indication of the schizophrenia-related disorder occurs spontaneously in the genetically modified animal.

48. The method of claim 42, wherein the incidence or indication of the schizophrenia-related disorder is promoted by exposure to a disruptive agent.

49. The method of claim 48, wherein the disruptive agent is chosen from a protein associated with schizophrenia, a drug, a toxin, a chemical, an activated retrovirus, and an environmental stress.


SEQUENCE LISTING (11 sequences)

SEQ ID NO: 1 - DNA, 200 bp, Rattus rattus
tacttcaagggctggcagcgatgacccagaagccccacttgagggacagcggaggaccaccgcccaggatagcctgccgggactagccgtcaccaggcgggactggctgatgcgagagaaagagcaattgcaggtgagcggtatagagtcttccagagagggagcggccctctcttatccgtgtgagtcagagcagagaa

SEQ ID NO: 2 - DNA, 400 bp, Rattus rattus
tcgaagagctgctggatgaggaccagaaggttcggcccaacgaagaaaaccataaggacgcggacttgtacacttcccgggtgatgctcagcagtcaagtgcctttggagcctcctctgctctttctgctggaggaatacaaaaattacctggatgccgcaaacatgtctatgagggttcggcgccactccgaccccgcccgccgtggggagctgagcgtgtgtgacagtattagcgagtgggtcacagcggcagataaaaagactgcagtggacatgtccggtgggacggtcacagtcctggagaaagtcccggtatcaaaaggccaactgaagcaatatttctacgagaccaagtgtaatcccatgggttacacgaaggaaggctgcaggggcataga

SEQ ID NO: 3 - DNA, 300 bp, Rattus rattus
gcctttggagcctcctctgctctttctgctggaggaatacaaaaattacctggatgccgcaaacatgtctatgagggttcggcgccactccgaccccgcccgccgtggggagctgagcgtgtgtgacagtattagcgagtgggtcacagcggcagataaaaagactgcagtggacatgtccggtgggacggtcacagtcctggagaaagtcccggtatcaaaaggccaactgaagcaatatttctacgagaccaagtgtaatcccatgggttacacgaaggaaggctgcaggggcataga

SEQ ID NO: 4 - DNA, 28 bp, Rattus rattus
tagtcccggcaggctatcctgggcggtg

SEQ ID NO: 5 - DNA, 28 bp, Rattus rattus
ccgtcaccaggcgggactggctgatgcg

SEQ ID NO: 6 - DNA, 28 bp, Rattus rattus
cggggtcggagtggcgccgaaccctcat

SEQ ID NO: 7 - DNA, 28 bp, Rattus rattus
ccgccgtggggagctgagcgtgtgtgac

SEQ ID NO: 8 - protein, 35 aa, Artificial Sequence
  SYNTHESIZED
NSGNLDKDRSHLSRQSGDLTRRSDTLSQDRSARTR

SEQ ID NO: 9 - protein, 35 aa, Artificial Sequence
  SYNTHESIZED
DRSNLSRRSDNLRERSDHLSADSSTRKTDRSSRKR

SEQ ID NO: 10 - protein, 35 aa, Artificial Sequence
  SYNTHESIZED
DRSDLSRDRSHLARRSHNLARRSDDLSKRSAHLSR

SEQ ID NO: 11 - protein, 35 aa, Artificial Sequence
  SYNTHESIZED
RSDNLARQSSDLRRRSSHLSRRSDALSRDRSDLSR